SEQUENCE LISTING (305 sequences)

SEQ ID NO: 1 - DNA, 3594 bp, Homo sapiens
gtggctcacctgtacccagcacttgggaagccgaggcgtgcagatcacctaagtcaggagttcgagaccagcccggccaacatggtgaaaccccgtctctactaaaaatacaaaaatcagccagatgtggcacgcacctatatccacctactcgggaggctgaagcagaatgcttaacccgagaggcggaggttgcagtgagccgcccagatcgcgccactgcactccagcctgggccacagcgtgagactactcataaaataaaataaaataaaataaaataaaataaaataaaataaaataataaaataaaataaaataaaataaaatataaaataaaataaaataaaataaaataaaataaaataaaataaaagcaatttcctttcctctaagcggcctccacccctctcccctgccctgtgaacgggggaagctccggatcgcagcaattagggaatttccccccgcgatgtcccggcgcgccagttcggcgcacatctttcgctgcggtcctcttcctgctgtctgtttactccctaggcccctggacctgggaaagagggaaaggcttcccgccagctgcgcggcgactccggggactccagggcgcccctctgcggcgacgcccgggtgcagcggccgccgggctggggccggcgggactccgcgggaccctccagaagagcggccggcggctgactcagcactggggcggaggggcgggacacccttataaggctcggagcgcgagccttcgctggagtttcgccgccgcagtcttcgccaccagtgagtacgcggccgcgtccccggggatggggctcagagctccagcatggggccaacccgcagcatcaggccgggctcccggcggcctccccacctcgagacccgggacggggcctaggggacccaggacgtcccagtgccgttagcggctttcagggggcccggagcgcctcggggagggatgggaccccgggggcgggagggcagctcactcaccgcgccttggcatcctccccgggctccacaaattttctttgttcgctgcagtgccgccctacaccgtggtctatttcccagttcgaggtaggagcatgtgtctggcagggaagggaggcaggggctggggctgcagcacccacagcccccacccggagagatccgaacccccttatccctcgtcgtgtgcttttacccccggcctccttcctgttccccgcctctcccgccatgcctgctccccgccccagtgttgtgtgaaatcttcggaggaacctgtttccctgttccctccctgcactcctgacccctccccgggttgctgcgaggcggagtcggcccggtccccacatctcgtacttctccctccccgcaggccgctgcgcggccctgcgcatgctgctggcagatcagggccagagctggaaggaggaggtggtgaccgtggagacgtggcaggagggctcactcaaagcctcctgcgtaagtgaccatgcccgggcaaggggagggggtgctgggccttagggggctgtgactaggatcgggggacgccccaagctcagtgcccctccctgagccatgcctcccccaacagctatacgggcagctccccaagttccaggacggagacctcaccctgtaccagtccaataccatcctgcgtcacctgggccgcacccttggtgagtcttgaacctccaagtccagggcaggcatgggcaagcctctgcccccggagcccttttgtttaaatcagctgccccgcagccctctggagtggaggaaactgagacccactgaggttacgtagtttgcccaaggtcaagcctgggtgcctgcaatccttgccctgtgccaggctgcctcccaggtgtcaggtgagctctgagcacctgctgtgtggcagtctctcatccttccacgcacatcctcttcccctcctcccaggctggggctcacagacagccccctggttggcccatccccagtgactgtgttgatcaggcgcccagtcacgcggcctgctcccctccacccaaccccagggctctatgggaaggaccagcaggaggcagccctggtggacatggtgaatgacggcgtggaggacctccgctgcaaatacatctccctcatctacaccaactatgtgagcatctgcaccagggttgggcactgggggctgaacaaagaaaggggcttcttgtgccctcaccccccttacccctcaggtggcttgggctgaccccttcttgggtcagggtgcaggggctgggtcagctctgggccaggggggcctgggacaagacacaacctgcacccttattgcctgggacatcaaccacccaagtaacgggtcatgggggcgagtgcaaggacagagacctccagcaactggtggtttctgctctcctggggtggccagaggtggaggaggatttgtgccagtttctggatggagccgctggcgcttttagctgaggaaaatatgagacacagagcactttgggtaccagggaccagttcagcagaggcagcgtgtgtggcgtgtgtgtgcgtgtgtgtgcgtgtgtgtgtgtacgcttgcatttgtgtcgggtgggtaaggagatagagatggggcggcagtaggcccaggtcccgaaggccttgaacccactggtttggagtctcctaagggcaatgggggccattgagaagtctgaacagggctgtgtctgaatgtgaggtctagaaggatcctccagagaagccagctctaaagcttttgcaatcatctggtgagagaacccagcaaggatggacaggcagaatggaatagagatgagttggcagctgaagtggacaggatttggtactagcctggttgtggggagcaagcagaggagaatctgggactctggtgtctggcctggggcagacgggggtgtctcaggggctgggagggatgagagtaggatgatacatggtgtgtgctggcaggaggcgggcaaggatgactatgtgaaggcactgcccgggcaactgaagccttttgagaccctgctgtcccagaaccagggaggcaagaccttcattgtgggagaccaggtgagcatctggccccatgctgttccttcctcgccaccctctgcttccagatggacacaggtgtgagccatttgtttagcaaagcagagcagacctaggggatgggcttaggccctctgcccccaattcctctccagcctgctcccgctggctgagtccctagcccccctgccctgcagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtgagggttggggggactctgagcgggaggcagagtttgccttcctttctccaggaccaataaaagggctaagagagctactatgagcactgtgtttcctgggacggggcttaggggttctcagcctc

SEQ ID NO: 2 - protein, 189 aa, Homo sapiens [sequence omitted]

SEQ ID NO: 3 - DNA, 21 bp, Artificial Sequence
  Description of Artificial Sequence Synthetic oligonucleotide
  Description of Combined DNA/RNA Molecule Synthetic oligonucleotide
ucccagaaccagggaggcatt

SEQ ID NO: 4 - DNA, 21 bp, Artificial Sequence
  Description of Artificial Sequence Synthetic oligonucleotide
  Description of Combined DNA/RNA Molecule Synthetic oligonucleotide
cuuuugagacccugcuguctt

SEQ ID NO: 5 - DNA, 21 bp, Artificial Sequence
  Description of Artificial Sequence Synthetic oligonucleotide
  Description of Combined DNA/RNA Molecule Synthetic oligonucleotide
cugucccagaaccagggagtt

SEQ ID NO: 6 - DNA, 21 bp, Artificial Sequence
  Description of Artificial Sequence Synthetic oligonucleotide
  Description of Combined DNA/RNA Molecule Synthetic oligonucleotide
ugucccagaaccagggaggtt

SEQ ID NO: 7 - DNA, 21 bp, Artificial Sequence
  Description of Artificial Sequence Synthetic oligonucleotide
  Description of Combined DNA/RNA Molecule Synthetic oligonucleotide
aagccuuuugagacccugctt

SEQ ID NO: 8 - DNA, 21 bp, Artificial Sequence
  Description of Artificial Sequence Synthetic oligonucleotide
  Description of Combined DNA/RNA Molecule Synthetic oligonucleotide
uugagacccugcugucccatt

SEQ ID NO: 9 - DNA, 21 bp, Artificial Sequence
  Description of Artificial Sequence Synthetic oligonucleotide
  Description of Combined DNA/RNA Molecule Synthetic oligonucleotide
uuuugagacccugcugucctt

SEQ ID NO: 10 - DNA, 21 bp, Artificial Sequence
  Description of Artificial Sequence Synthetic oligonucleotide
  Description of Combined DNA/RNA Molecule Synthetic oligonucleotide
gagacccugcugucccagatt

SEQ ID NO: 11 - DNA, 21 bp, Artificial Sequence
  Description of Artificial Sequence Synthetic oligonucleotide
  Description of Combined DNA/RNA Molecule Synthetic oligonucleotide
gcuggaaggaggagguggutt

SEQ ID NO: 12 - DNA, 21 bp, Artificial Sequence
  Description of Artificial Sequence Synthetic oligonucleotide
  Description of Combined DNA/RNA Molecule Synthetic oligonucleotide
cuggaaggaggagguggugtt

SEQ ID NO: 13 - DNA, 21 bp, Artificial Sequence
  Description of Artificial Sequence Synthetic oligonucleotide
  Description of Combined DNA/RNA Molecule Synthetic oligonucleotide
ucagggccagagcuggaagtt

SEQ ID NO: 14 - DNA, 21 bp, Artificial Sequence
  Description of Artificial Sequence Synthetic oligonucleotide
  Description of Combined DNA/RNA Molecule Synthetic oligonucleotide
ugagacccugcugucccagtt

SEQ ID NO: 15 - DNA, 21 bp, Artificial Sequence
  Description of Artificial Sequence Synthetic oligonucleotide
  Description of Combined DNA/RNA Molecule Synthetic oligonucleotide
agggccagagcuggaaggatt

SEQ ID NO: 16 - DNA, 21 bp, Artificial Sequence
  Description of Artificial Sequence Synthetic oligonucleotide
  Description of Combined DNA/RNA Molecule Synthetic oligonucleotide
agcuggaaggaggagguggtt

SEQ ID NO: 17 - DNA, 21 bp, Artificial Sequence
  Description of Artificial Sequence Synthetic oligonucleotide
  Description of Combined DNA/RNA Molecule Synthetic oligonucleotide
agacccugcugucccagaatt

SEQ ID NO: 18 - DNA, 21 bp, Artificial Sequence
  Description of Artificial Sequence Synthetic oligonucleotide
  Description of Combined DNA/RNA Molecule Synthetic oligonucleotide
gagcuggaaggaggaggugtt

SEQ ID NO: 19 - DNA, 21 bp, Artificial Sequence
  Description of Artificial Sequence Synthetic oligonucleotide
  Description of Combined DNA/RNA Molecule Synthetic oligonucleotide
ugcugucccagaaccagggtt

SEQ ID NO: 20 - DNA, 21 bp, Artificial Sequence
  Description of Artificial Sequence Synthetic oligonucleotide
  Description of Combined DNA/RNA Molecule Synthetic oligonucleotide
cccagaaccagggaggcaatt

SEQ ID NO: 21 - DNA, 21 bp, Artificial Sequence
  Description of Artificial Sequence Synthetic oligonucleotide
  Description of Combined DNA/RNA Molecule Synthetic oligonucleotide
ccagaaccagggaggcaagtt

SEQ ID NO: 22 - DNA, 21 bp, Artificial Sequence
  Description of Artificial Sequence Synthetic oligonucleotide
  Description of Combined DNA/RNA Molecule Synthetic oligonucleotide
uuugagacccugcuguccctt

SEQ ID NO: 23 - DNA, 21 bp, Artificial Sequence
  Description of Artificial Sequence Synthetic oligonucleotide
  Description of Combined DNA/RNA Molecule Synthetic oligonucleotide
gacccugcugucccagaactt

SEQ ID NO: 24 - DNA, 21 bp, Artificial Sequence
  Description of Artificial Sequence Synthetic oligonucleotide
  Description of Combined DNA/RNA Molecule Synthetic oligonucleotide
gaucagggccagagcuggatt

SEQ ID NO: 25 - DNA, 21 bp, Artificial Sequence
  Description of Artificial Sequence Synthetic oligonucleotide
  Description of Combined DNA/RNA Molecule Synthetic oligonucleotide
agccuuuugagacccugcutt

SEQ ID NO: 26 - DNA, 21 bp, Artificial Sequence
  Description of Artificial Sequence Synthetic oligonucleotide
  Description of Combined DNA/RNA Molecule Synthetic oligonucleotide
gccuuuugagacccugcugtt

SEQ ID NO: 27 - DNA, 21 bp, Artificial Sequence
  Description of Artificial Sequence Synthetic oligonucleotide
  Description of Combined DNA/RNA Molecule Synthetic oligonucleotide
ccuuuugagacccugcugutt

SEQ ID NO: 28 - DNA, 21 bp, Artificial Sequence
  Description of Artificial Sequence Synthetic oligonucleotide
  Description of Combined DNA/RNA Molecule Synthetic oligonucleotide
cgccuuuugagacccugcatt

SEQ ID NO: 29 - DNA, 21 bp, Artificial Sequence
  Description of Artificial Sequence Synthetic oligonucleotide
  Description of Combined DNA/RNA Molecule Synthetic oligonucleotide
ccuacaccguggucuauuutt

SEQ ID NO: 30 - DNA, 21 bp, Artificial Sequence
  Description of Artificial Sequence Synthetic oligonucleotide
  Description of Combined DNA/RNA Molecule Synthetic oligonucleotide
ugugggagaccagaucucctt

SEQ ID NO: 31 - DNA, 21 bp, Artificial Sequence
  Description of Artificial Sequence Synthetic oligonucleotide
  Description of Combined DNA/RNA Molecule Synthetic oligonucleotide
gcgggaggcagaguuugcctt

SEQ ID NO: 32 - DNA, 21 bp, Artificial Sequence
  Description of Artificial Sequence Synthetic oligonucleotide
  Description of Combined DNA/RNA Molecule Synthetic oligonucleotide
ccuuucuccaggaccaauatt

SEQ ID NO: 33 - DNA, 21 bp, Artificial Sequence
  Description of Artificial Sequence Synthetic oligonucleotide
  Description of Combined DNA/RNA Molecule Synthetic oligonucleotide
acccugcugucccagaacctt

SEQ ID NO: 34 - DNA, 21 bp, Artificial Sequence
  Description of Artificial Sequence Synthetic oligonucleotide
  Description of Combined DNA/RNA Molecule Synthetic oligonucleotide
ggucuauuucccaguucgatt

SEQ ID NO: 35 - DNA, 21 bp, Artificial Sequence
  Description of Artificial Sequence Synthetic oligonucleotide
  Description of Combined DNA/RNA Molecule Synthetic oligonucleotide
cccugguggacauggugaatt

SEQ ID NO: 36 - DNA, 21 bp, Artificial Sequence
  Description of Artificial Sequence Synthetic oligonucleotide
  Description of Combined DNA/RNA Molecule Synthetic oligonucleotide
acaucucccucaucuacactt

SEQ ID NO: 37 - DNA, 21 bp, Artificial Sequence
  Description of Artificial Sequence Synthetic oligonucleotide
  Description of Combined DNA/RNA Molecule Synthetic oligonucleotide
gcaaggaugacuaugugaatt

SEQ ID NO: 38 - DNA, 21 bp, Artificial Sequence
  Description of Artificial Sequence Synthetic oligonucleotide
  Description of Combined DNA/RNA Molecule Synthetic oligonucleotide
ccuucgcugacuacaaccutt

SEQ ID NO: 39 - DNA, 21 bp, Artificial Sequence
  Description of Artificial Sequence Synthetic oligonucleotide
  Description of Combined DNA/RNA Molecule Synthetic oligonucleotide
cuggcagaucagggccagatt

SEQ ID NO: 40 - DNA, 21 bp, Artificial Sequence
  Description of Artificial Sequence Synthetic oligonucleotide
  Description of Combined DNA/RNA Molecule Synthetic oligonucleotide
gacggagaccucacccugutt

SEQ ID NO: 41 - DNA, 21 bp, Artificial Sequence
  Description of Artificial Sequence Synthetic oligonucleotide
  Description of Combined DNA/RNA Molecule Synthetic oligonucleotide
cgggcaaggaugacuaugutt

SEQ ID NO: 42 - DNA, 21 bp, Artificial Sequence
  Description of Artificial Sequence Synthetic oligonucleotide
  Description of Combined DNA/RNA Molecule Synthetic oligonucleotide
cuuuugagacccugcuguatt

SEQ ID NO: 43 - DNA, 21 bp, Artificial Sequence
  Description of Artificial Sequence Synthetic oligonucleotide
  Description of Combined DNA/RNA Molecule Synthetic oligonucleotide
gagcuggaaggaggagguatt

SEQ ID NO: 44 - DNA, 21 bp, Artificial Sequence
  Description of Artificial Sequence Synthetic oligonucleotide
  Description of Combined DNA/RNA Molecule Synthetic oligonucleotide
acccugcugucccagaacatt

SEQ ID NO: 45 - DNA, 21 bp, Artificial Sequence
  Description of Artificial Sequence Synthetic oligonucleotide
  Description of Combined DNA/RNA Molecule Synthetic oligonucleotide
ugcugucccagaaccaggatt

SEQ ID NO: 46 - DNA, 21 bp, Artificial Sequence
  Description of Artificial Sequence Synthetic oligonucleotide
  Description of Combined DNA/RNA Molecule Synthetic oligonucleotide
agccuuuugagacccugcatt

SEQ ID NO: 47 - DNA, 21 bp, Artificial Sequence
  Description of Artificial Sequence Synthetic oligonucleotide
  Description of Combined DNA/RNA Molecule Synthetic oligonucleotide
ccuuuugagacccugcugatt

SEQ ID NO: 48 - DNA, 21 bp, Artificial Sequence
  Description of Artificial Sequence Synthetic oligonucleotide
  Description of Combined DNA/RNA Molecule Synthetic oligonucleotide
ugaagccuuuugagacccutt

SEQ ID NO: 49 - DNA, 21 bp, Artificial Sequence
  Description of Artificial Sequence Synthetic oligonucleotide
  Description of Combined DNA/RNA Molecule Synthetic oligonucleotide
acugaagccuuuugagacctt

SEQ ID NO: 50 - DNA, 21 bp, Artificial Sequence
  Description of Artificial Sequence Synthetic oligonucleotide
  Description of Combined DNA/RNA Molecule Synthetic oligonucleotide
  modified_base (20)..(21): 2′-deoxy-nucleotide
aaggaugacuaugugaaggtt

SEQ ID NO: 51 - DNA, 21 bp, Artificial Sequence
  Description of Artificial Sequence Synthetic oligonucleotide
  Description of Combined DNA/RNA Molecule Synthetic oligonucleotide
  modified_base (20)..(21): 2′-deoxy-nucleotide
aggaugacuaugugaaggctt

SEQ ID NO: 52 - DNA, 21 bp, Artificial Sequence
  Description of Artificial Sequence Synthetic oligonucleotide
  Description of Combined DNA/RNA Molecule Synthetic oligonucleotide
  modified_base (20)..(21): 2′-deoxy-nucleotide
ggaugacuaugugaaggcatt

SEQ ID NO: 53 - DNA, 21 bp, Artificial Sequence
  Description of Artificial Sequence Synthetic oligonucleotide
  Description of Combined DNA/RNA Molecule Synthetic oligonucleotide
cucccucaucuacaccaactt

SEQ ID NO: 54 - DNA, 21 bp, Artificial Sequence
  Description of Artificial Sequence Synthetic oligonucleotide
  Description of Combined DNA/RNA Molecule Synthetic oligonucleotide
gaagccuuuugagacccugtt

SEQ ID NO: 55 - DNA, 21 bp, Artificial Sequence
  Description of Artificial Sequence Synthetic oligonucleotide
  Description of Combined DNA/RNA Molecule Synthetic oligonucleotide
ucucccucaucuacaccaatt

SEQ ID NO: 56 - DNA, 21 bp, Artificial Sequence
  Description of Artificial Sequence Synthetic oligonucleotide
  Description of Combined DNA/RNA Molecule Synthetic oligonucleotide
ccucaucuacaccaacuautt

SEQ ID NO: 57 - DNA, 21 bp, Artificial Sequence
  Description of Artificial Sequence Synthetic oligonucleotide
  Description of Combined DNA/RNA Molecule Synthetic oligonucleotide
cccucaucuacaccaacuatt

SEQ ID NO: 58 - DNA, 21 bp, Artificial Sequence
  Description of Artificial Sequence Synthetic oligonucleotide
  Description of Combined DNA/RNA Molecule Synthetic oligonucleotide
caacugaagccuuuugagatt

SEQ ID NO: 59 - DNA, 21 bp, Artificial Sequence
  Description of Artificial Sequence Synthetic oligonucleotide
  Description of Combined DNA/RNA Molecule Synthetic oligonucleotide
aacugaagccuuuugagactt

SEQ ID NO: 60 - DNA, 21 bp, Artificial Sequence
  Description of Artificial Sequence Synthetic oligonucleotide
  Description of Combined DNA/RNA Molecule Synthetic oligonucleotide
cugaagccuuuugagaccctt

SEQ ID NO: 61 - DNA, 21 bp, Artificial Sequence
  Description of Artificial Sequence Synthetic oligonucleotide
  Description of Combined DNA/RNA Molecule Synthetic oligonucleotide
ucccucaucuacaccaacutt

SEQ ID NO: 62 - DNA, 21 bp, Artificial Sequence
  Description of Artificial Sequence Synthetic oligonucleotide
  Description of Combined DNA/RNA Molecule Synthetic oligonucleotide
gcucccucaucuacaccaatt

SEQ ID NO: 63 - DNA, 21 bp, Artificial Sequence
  Description of Artificial Sequence Synthetic oligonucleotide
  Description of Combined DNA/RNA Molecule Synthetic oligonucleotide
gaagccuuuugagacccuatt

SEQ ID NO: 64 - DNA, 21 bp, Artificial Sequence
  Description of Artificial Sequence Synthetic oligonucleotide
  Description of Combined DNA/RNA Molecule Synthetic oligonucleotide
acugaagccuuuugagacatt

SEQ ID NO: 65 - DNA, 21 bp, Artificial Sequence
  Description of Artificial Sequence Synthetic oligonucleotide
  Description of Combined DNA/RNA Molecule Synthetic oligonucleotide
cucccucaucuacaccaaatt

SEQ ID NO: 66 - DNA, 21 bp, Artificial Sequence
  Description of Artificial Sequence Synthetic oligonucleotide
  Description of Combined DNA/RNA Molecule Synthetic oligonucleotide
ccucaucuacaccaacuaatt

SEQ ID NO: 67 - DNA, 21 bp, Artificial Sequence
  Description of Artificial Sequence Synthetic oligonucleotide
  Description of Combined DNA/RNA Molecule Synthetic oligonucleotide
accaauaaaauuucuaagatt

SEQ ID NO: 68 - DNA, 21 bp, Artificial Sequence
  Description of Artificial Sequence Synthetic oligonucleotide
  Description of Combined DNA/RNA Molecule Synthetic oligonucleotide
ugccucccugguucugggaca

SEQ ID NO: 69 - DNA, 21 bp, Artificial Sequence
  Description of Artificial Sequence Synthetic oligonucleotide
  Description of Combined DNA/RNA Molecule Synthetic oligonucleotide
gacagcagggucucaaaaggc

SEQ ID NO: 70 - DNA, 21 bp, Artificial Sequence
  Description of Artificial Sequence Synthetic oligonucleotide
  Description of Combined DNA/RNA Molecule Synthetic oligonucleotide
cucccugguucugggacagca

SEQ ID NO: 71 - DNA, 21 bp, Artificial Sequence
  Description of Artificial Sequence Synthetic oligonucleotide
  Description of Combined DNA/RNA Molecule Synthetic oligonucleotide
ccucccugguucugggacagc

SEQ ID NO: 72 - DNA, 21 bp, Artificial Sequence
  Description of Artificial Sequence Synthetic oligonucleotide
  Description of Combined DNA/RNA Molecule Synthetic oligonucleotide
gcagggucucaaaaggcuuca

SEQ ID NO: 73 - DNA, 21 bp, Artificial Sequence
  Description of Artificial Sequence Synthetic oligonucleotide
  Description of Combined DNA/RNA Molecule Synthetic oligonucleotide
ugggacagcagggucucaaaa

SEQ ID NO: 74 - DNA, 21 bp, Artificial Sequence
  Description of Artificial Sequence Synthetic oligonucleotide
  Description of Combined DNA/RNA Molecule Synthetic oligonucleotide
ggacagcagggucucaaaagg

SEQ ID NO: 75 - DNA, 21 bp, Artificial Sequence
  Description of Artificial Sequence Synthetic oligonucleotide
  Description of Combined DNA/RNA Molecule Synthetic oligonucleotide
ucugggacagcagggucucaa

SEQ ID NO: 76 - DNA, 21 bp, Artificial Sequence
  Description of Artificial Sequence Synthetic oligonucleotide
  Description of Combined DNA/RNA Molecule Synthetic oligonucleotide
accaccuccuccuuccagctc

SEQ ID NO: 77 - DNA, 21 bp, Artificial Sequence
  Description of Artificial Sequence Synthetic oligonucleotide
  Description of Combined DNA/RNA Molecule Synthetic oligonucleotide
caccaccuccuccuuccagct

SEQ ID NO: 78 - DNA, 21 bp, Artificial Sequence
  Description of Artificial Sequence Synthetic oligonucleotide
  Description of Combined DNA/RNA Molecule Synthetic oligonucleotide
cuuccagcucuggcccugatc

SEQ ID NO: 79 - DNA, 21 bp, Artificial Sequence
  Description of Artificial Sequence Synthetic oligonucleotide
  Description of Combined DNA/RNA Molecule Synthetic oligonucleotide
cugggacagcagggucucaaa

SEQ ID NO: 80 - DNA, 21 bp, Artificial Sequence
  Description of Artificial Sequence Synthetic oligonucleotide
  Description of Combined DNA/RNA Molecule Synthetic oligonucleotide
uccuuccagcucuggcccuga

SEQ ID NO: 81 - DNA, 21 bp, Artificial Sequence
  Description of Artificial Sequence Synthetic oligonucleotide
  Description of Combined DNA/RNA Molecule Synthetic oligonucleotide
ccaccuccuccuuccagcuct

SEQ ID NO: 82 - DNA, 21 bp, Artificial Sequence
  Description of Artificial Sequence Synthetic oligonucleotide
  Description of Combined DNA/RNA Molecule Synthetic oligonucleotide
uucugggacagcagggucuca

SEQ ID NO: 83 - DNA, 21 bp, Artificial Sequence
  Description of Artificial Sequence Synthetic oligonucleotide
  Description of Combined DNA/RNA Molecule Synthetic oligonucleotide
caccuccuccuuccagcuctg

SEQ ID NO: 84 - DNA, 21 bp, Artificial Sequence
  Description of Artificial Sequence Synthetic oligonucleotide
  Description of Combined DNA/RNA Molecule Synthetic oligonucleotide
cccugguucugggacagcagg

SEQ ID NO: 85 - DNA, 21 bp, Artificial Sequence
  Description of Artificial Sequence Synthetic oligonucleotide
  Description of Combined DNA/RNA Molecule Synthetic oligonucleotide
uugccucccugguucugggac

SEQ ID NO: 86 - DNA, 21 bp, Artificial Sequence
  Description of Artificial Sequence Synthetic oligonucleotide
  Description of Combined DNA/RNA Molecule Synthetic oligonucleotide
cuugccucccugguucuggga

SEQ ID NO: 87 - DNA, 21 bp, Artificial Sequence
  Description of Artificial Sequence Synthetic oligonucleotide
  Description of Combined DNA/RNA Molecule Synthetic oligonucleotide
gggacagcagggucucaaaag

SEQ ID NO: 88 - DNA, 21 bp, Artificial Sequence
  Description of Artificial Sequence Synthetic oligonucleotide
  Description of Combined DNA/RNA Molecule Synthetic oligonucleotide
guucugggacagcaggguctc

SEQ ID NO: 89 - DNA, 21 bp, Artificial Sequence
  Description of Artificial Sequence Synthetic oligonucleotide
  Description of Combined DNA/RNA Molecule Synthetic oligonucleotide
uccagcucuggcccugauctg

SEQ ID NO: 90 - DNA, 21 bp, Artificial Sequence
  Description of Artificial Sequence Synthetic oligonucleotide
  Description of Combined DNA/RNA Molecule Synthetic oligonucleotide
agcagggucucaaaaggcutc

SEQ ID NO: 91 - DNA, 21 bp, Artificial Sequence
  Description of Artificial Sequence Synthetic oligonucleotide
  Description of Combined DNA/RNA Molecule Synthetic oligonucleotide
cagcagggucucaaaaggctt

SEQ ID NO: 92 - DNA, 21 bp, Artificial Sequence
  Description of Artificial Sequence Synthetic oligonucleotide
  Description of Combined DNA/RNA Molecule Synthetic oligonucleotide
acagcagggucucaaaaggct

SEQ ID NO: 93 - DNA, 21 bp, Artificial Sequence
  Description of Artificial Sequence Synthetic oligonucleotide
  Description of Combined DNA/RNA Molecule Synthetic oligonucleotide
ugcagggucucaaaaggcgtc

SEQ ID NO: 94 - DNA, 21 bp, Artificial Sequence
  Description of Artificial Sequence Synthetic oligonucleotide
  Description of Combined DNA/RNA Molecule Synthetic oligonucleotide
aaauagaccacgguguagggc

SEQ ID NO: 95 - DNA, 21 bp, Artificial Sequence
  Description of Artificial Sequence Synthetic oligonucleotide
  Description of Combined DNA/RNA Molecule Synthetic oligonucleotide
ggagaucuggucucccacaat

SEQ ID NO: 96 - DNA, 21 bp, Artificial Sequence
  Description of Artificial Sequence Synthetic oligonucleotide
  Description of Combined DNA/RNA Molecule Synthetic oligonucleotide
ggcaaacucugccucccgctc

SEQ ID NO: 97 - DNA, 21 bp, Artificial Sequence
  Description of Artificial Sequence Synthetic oligonucleotide
  Description of Combined DNA/RNA Molecule Synthetic oligonucleotide
uauugguccuggagaaaggaa

SEQ ID NO: 98 - DNA, 21 bp, Artificial Sequence
  Description of Artificial Sequence Synthetic oligonucleotide
  Description of Combined DNA/RNA Molecule Synthetic oligonucleotide
gguucugggacagcaggguct

SEQ ID NO: 99 - DNA, 21 bp, Artificial Sequence
  Description of Artificial Sequence Synthetic oligonucleotide
  Description of Combined DNA/RNA Molecule Synthetic oligonucleotide
ucgaacugggaaauagaccac

SEQ ID NO: 100 - DNA, 21 bp, Artificial Sequence
  Description of Artificial Sequence Synthetic oligonucleotide
  Description of Combined DNA/RNA Molecule Synthetic oligonucleotide
uucaccauguccaccagggct

SEQ ID NO: 101 - DNA, 21 bp, Artificial Sequence
  Description of Artificial Sequence Synthetic oligonucleotide
  Description of Combined DNA/RNA Molecule Synthetic oligonucleotide
guguagaugagggagauguat

SEQ ID NO: 102 - DNA, 21 bp, Artificial Sequence
  Description of Artificial Sequence Synthetic oligonucleotide
  Description of Combined DNA/RNA Molecule Synthetic oligonucleotide
uucacauagucauccuugccc

SEQ ID NO: 103 - DNA, 21 bp, Artificial Sequence
  Description of Artificial Sequence Synthetic oligonucleotide
  Description of Combined DNA/RNA Molecule Synthetic oligonucleotide
agguuguagucagcgaaggag

SEQ ID NO: 104 - DNA, 21 bp, Artificial Sequence
  Description of Artificial Sequence Synthetic oligonucleotide
  Description of Combined DNA/RNA Molecule Synthetic oligonucleotide
ucuggcccugaucugccagca

SEQ ID NO: 105 - DNA, 21 bp, Artificial Sequence
  Description of Artificial Sequence Synthetic oligonucleotide
  Description of Combined DNA/RNA Molecule Synthetic oligonucleotide
acagggugaggucuccgucct

SEQ ID NO: 106 - DNA, 21 bp, Artificial Sequence
  Description of Artificial Sequence Synthetic oligonucleotide
  Description of Combined DNA/RNA Molecule Synthetic oligonucleotide
acauagucauccuugcccgcc

SEQ ID NO: 107 - DNA, 21 bp, Artificial Sequence
  Description of Artificial Sequence Synthetic oligonucleotide
  Description of Combined DNA/RNA Molecule Synthetic oligonucleotide
uacagcagggucucaaaaggc

SEQ ID NO: 108 - DNA, 21 bp, Artificial Sequence
  Description of Artificial Sequence Synthetic oligonucleotide
  Description of Combined DNA/RNA Molecule Synthetic oligonucleotide
uaccuccuccuuccagcuctg

SEQ ID NO: 109 - DNA, 21 bp, Artificial Sequence
  Description of Artificial Sequence Synthetic oligonucleotide
  Description of Combined DNA/RNA Molecule Synthetic oligonucleotide
uguucugggacagcaggguct

SEQ ID NO: 110 - DNA, 21 bp, Artificial Sequence
  Description of Artificial Sequence Synthetic oligonucleotide
  Description of Combined DNA/RNA Molecule Synthetic oligonucleotide
uccugguucugggacagcagg

SEQ ID NO: 111 - DNA, 21 bp, Artificial Sequence
  Description of Artificial Sequence Synthetic oligonucleotide
  Description of Combined DNA/RNA Molecule Synthetic oligonucleotide
ugcagggucucaaaaggcutc

SEQ ID NO: 112 - DNA, 21 bp, Artificial Sequence
  Description of Artificial Sequence Synthetic oligonucleotide
  Description of Combined DNA/RNA Molecule Synthetic oligonucleotide
ucagcagggucucaaaaggct

SEQ ID NO: 113 - DNA, 21 bp, Artificial Sequence
  Description of Artificial Sequence Synthetic oligonucleotide
  Description of Combined DNA/RNA Molecule Synthetic oligonucleotide
agggucucaaaaggcuucagt

SEQ ID NO: 114 - DNA, 21 bp, Artificial Sequence
  Description of Artificial Sequence Synthetic oligonucleotide
  Description of Combined DNA/RNA Molecule Synthetic oligonucleotide
ggucucaaaaggcuucagutg

SEQ ID NO: 115 - DNA, 21 bp, Artificial Sequence
  Description of Artificial Sequence Synthetic oligonucleotide
  Description of Combined DNA/RNA Molecule Synthetic oligonucleotide
  modified_base (20)..(21): 2′-deoxy-nucleotide
ccuucacauagucauccuugc

SEQ ID NO: 116 - DNA, 21 bp, Artificial Sequence
  Description of Artificial Sequence Synthetic oligonucleotide
  Description of Combined DNA/RNA Molecule Synthetic oligonucleotide
  modified_base (20)..(21): 2′-deoxy-nucleotide
gccuucacauagucauccutg

SEQ ID NO: 117 - DNA, 21 bp, Artificial Sequence
  Description of Artificial Sequence Synthetic oligonucleotide
  Description of Combined DNA/RNA Molecule Synthetic oligonucleotide
  modified_base (20)..(21): 2′-deoxy-nucleotide
ugccuucacauagucaucctt

SEQ ID NO: 118 - DNA, 21 bp, Artificial Sequence
  Description of Artificial Sequence Synthetic oligonucleotide
  Description of Combined DNA/RNA Molecule Synthetic oligonucleotide
guugguguagaugagggagat

SEQ ID NO: 119 - DNA, 21 bp, Artificial Sequence
  Description of Artificial Sequence Synthetic oligonucleotide
  Description of Combined DNA/RNA Molecule Synthetic oligonucleotide
cagggucucaaaaggcuucag

SEQ ID NO: 120 - DNA, 21 bp, Artificial Sequence
  Description of Artificial Sequence Synthetic oligonucleotide
  Description of Combined DNA/RNA Molecule Synthetic oligonucleotide
uugguguagaugagggagatg

SEQ ID NO: 121 - DNA, 21 bp, Artificial Sequence
  Description of Artificial Sequence Synthetic oligonucleotide
  Description of Combined DNA/RNA Molecule Synthetic oligonucleotide
auaguugguguagaugaggga

SEQ ID NO: 122 - DNA, 21 bp, Artificial Sequence
  Description of Artificial Sequence Synthetic oligonucleotide
  Description of Combined DNA/RNA Molecule Synthetic oligonucleotide
uaguugguguagaugagggag

SEQ ID NO: 123 - DNA, 21 bp, Artificial Sequence
  Description of Artificial Sequence Synthetic oligonucleotide
  Description of Combined DNA/RNA Molecule Synthetic oligonucleotide
ucucaaaaggcuucaguugcc

SEQ ID NO: 124 - DNA, 21 bp, Artificial Sequence
  Description of Artificial Sequence Synthetic oligonucleotide
  Description of Combined DNA/RNA Molecule Synthetic oligonucleotide
gucucaaaaggcuucaguugc

SEQ ID NO: 125 - DNA, 21 bp, Artificial Sequence
  Description of Artificial Sequence Synthetic oligonucleotide
  Description of Combined DNA/RNA Molecule Synthetic oligonucleotide
gggucucaaaaggcuucagtt

SEQ ID NO: 126 - DNA, 21 bp, Artificial Sequence
  Description of Artificial Sequence Synthetic oligonucleotide
  Description of Combined DNA/RNA Molecule Synthetic oligonucleotide
aguugguguagaugagggaga

SEQ ID NO: 127 - DNA, 21 bp, Artificial Sequence
  Description of Artificial Sequence Synthetic oligonucleotide
  Description of Combined DNA/RNA Molecule Synthetic oligonucleotide
uugguguagaugagggagctg

SEQ ID NO: 128 - DNA, 21 bp, Artificial Sequence
  Description of Artificial Sequence Synthetic oligonucleotide
  Description of Combined DNA/RNA Molecule Synthetic oligonucleotide
uagggucucaaaaggcuucag

SEQ ID NO: 129 - DNA, 21 bp, Artificial Sequence
  Description of Artificial Sequence Synthetic oligonucleotide
  Description of Combined DNA/RNA Molecule Synthetic oligonucleotide
ugucucaaaaggcuucagutg

SEQ ID NO: 130 - DNA, 21 bp, Artificial Sequence
  Description of Artificial Sequence Synthetic oligonucleotide
  Description of Combined DNA/RNA Molecule Synthetic oligonucleotide
uuugguguagaugagggagat

SEQ ID NO: 131 - DNA, 21 bp, Artificial Sequence
  Description of Artificial Sequence Synthetic oligonucleotide
  Description of Combined DNA/RNA Molecule Synthetic oligonucleotide
uuaguugguguagaugaggga

SEQ ID NO: 132 - DNA, 21 bp, Artificial Sequence
  Description of Artificial Sequence Synthetic oligonucleotide
  Description of Combined DNA/RNA Molecule Synthetic oligonucleotide
ucuuagaaauuuuauuggucc

SEQ ID NO: 133 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
gaagccuuuugagacccuann

SEQ ID NO: 134 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 135 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 136 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 137 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 138 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 139 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 140 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 141 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 142 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 143 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 144 - DNA, 21 bp, Artificial Sequence
  Description of Artificial Sequence Synthetic oligonucleotide
  Description of Combined DNA/RNA Molecule Synthetic oligonucleotide
gaagccuuuugagacccuatt

SEQ ID NO: 145 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 146 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 147 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 148 - RNA, 21 nt, Artificial Sequence
  Description of Artificial Sequence Synthetic oligonucleotide
  modified_base (20)..(21): 2′-OMe-nucleotide
gaagccuuuugagacccuauu

SEQ ID NO: 149 - RNA, 21 nt, Artificial Sequence
  Description of Artificial Sequence Synthetic oligonucleotide
  modified_base (19)..(19): 2′-deoxy-2′-fluoro-nucleotide
  modified_base (20)..(21): 2′-OMe-nucleotide
gaagccuuuugagacccuauu

SEQ ID NO: 150 - RNA, 21 nt, Artificial Sequence
  Description of Artificial Sequence Synthetic oligonucleotide
  modified_base (14)..(14): 2′-deoxy-2′-fluoro-nucleotide
  modified_base (19)..(19): 2′-deoxy-2′-fluoro-nucleotide
  modified_base (20)..(21): 2′-OMe-nucleotide
gaagccuuuugagacccuauu

SEQ ID NO: 151 - RNA, 21 nt, Artificial Sequence
  Description of Artificial Sequence Synthetic oligonucleotide
  modified_base (19)..(21): 2′-OMe-nucleotide
gaagccuuuugagacccuauu

SEQ ID NO: 152 - RNA, 21 nt, Artificial Sequence
  Description of Artificial Sequence Synthetic oligonucleotide
  modified_base (19)..(21): 2′-OMe-nucleotide
gaagccuuuugagacccuauu

SEQ ID NO: 153 - RNA, 21 nt, Artificial Sequence
  Description of Artificial Sequence Synthetic oligonucleotide
  modified_base (1)..(3): 2′-OMe-nucleotide
  modified_base (20)..(21): 2′-OMe-nucleotide
gaagccuuuugagacccuauu

SEQ ID NO: 154 - RNA, 21 nt, Artificial Sequence
  Description of Artificial Sequence Synthetic oligonucleotide
  modified_base (1)..(3): 2′-OMe-nucleotide
  modified_base (8)..(8): 2′-OMe-nucleotide
  modified_base (10)..(10): 2′-OMe-nucleotide
  modified_base (18)..(18): 2′-OMe-nucleotide
  modified_base (20)..(21): 2′-OMe-nucleotide
gaagccuuuugagacccuauu

SEQ ID NO: 155 - RNA, 21 nt, Artificial Sequence
  Description of Artificial Sequence Synthetic oligonucleotide
  modified_base (1)..(3): 2′-OMe-nucleotide
  modified_base (20)..(21): 2′-OMe-nucleotide
gaagccuuuugagacccuauu

SEQ ID NO: 156 - RNA, 21 nt, Artificial Sequence
  Description of Artificial Sequence Synthetic oligonucleotide
  modified_base (1)..(3): 2′-OMe-nucleotide
  modified_base (8)..(8): 2′-OMe-nucleotide
  modified_base (10)..(10): 2′-OMe-nucleotide
  modified_base (18)..(18): 2′-OMe-nucleotide
  modified_base (20)..(21): 2′-OMe-nucleotide
gaagccuuuugagacccuauu

SEQ ID NO: 157 - RNA, 21 nt, Artificial Sequence
  Description of Artificial Sequence Synthetic oligonucleotide
  modified_base (1)..(3): 2′-OMe-nucleotide
  modified_base (8)..(8): 2′-OMe-nucleotide
  modified_base (10)..(10): 2′-OMe-nucleotide
  modified_base (18)..(21): 2′-OMe-nucleotide
gaagccuuuugagacccuauu

SEQ ID NO: 158 - RNA, 21 nt, Artificial Sequence
  Description of Artificial Sequence Synthetic oligonucleotide
  modified_base (1)..(3): 2′-OMe-nucleotide
  modified_base (8)..(8): 2′-OMe-nucleotide
  modified_base (10)..(10): 2′-OMe-nucleotide
  modified_base (18)..(21): 2′-OMe-nucleotide
gaagccuuuugagacccuauu

SEQ ID NO: 159 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
uagggucucaaaaggcuucnn

SEQ ID NO: 160 - RNA, 21 nt, Artificial Sequence
  Description of Artificial Sequence Synthetic oligonucleotide
  modified_base (20)..(21): 2′-OMe-nucleotide
uagggucucaaaaggcuucuu

SEQ ID NO: 161 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-deoxy-nucleotide
  modified_base (6)..(6): 2′-deoxy-nucleotide
  modified_base (8)..(8): 2′-deoxy-nucleotide
  modified_base (20)..(21): 2′-OMe-nucleotide
uagggucucaaaaggcuucuu

SEQ ID NO: 162 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
uagggucucaaaaggcuucuu

SEQ ID NO: 163 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
uagggucucaaaaggcuucuu

SEQ ID NO: 164 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
uagggucucaaaaggcuucuu

SEQ ID NO: 165 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
uagggucucaaaaggcuucuu

SEQ ID NO: 166 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-deoxy-nucleotide
  modified_base (20)..(21): 2′-OMe-nucleotide
uagggucucaaaaggcuucuu

SEQ ID NO: 167 - DNA, 21 bp, Artificial Sequence
  Description of Artificial Sequence Synthetic oligonucleotide
  Description of Combined DNA/RNA Molecule Synthetic oligonucleotide
  modified_base (20)..(21): 2′-OMe-nucleotide
uagggucucaaaaggcuucuu

SEQ ID NO: 168 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
uagggucucaaaaggcuucuu

SEQ ID NO: 169 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
uagggucucaaaaggcuucuu

SEQ ID NO: 170 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (14)..(14): 2′-OMe-nucleotide
  modified_base (16)..(16): 2′-OMe-nucleotide
  modified_base (18)..(18): 2′-OMe-nucleotide
  modified_base (20)..(21): 2′-OMe-nucleotide
uagggucucaaaaggcuucuu

SEQ ID NO: 171 - RNA, 21 nt, Artificial Sequence
  Description of Artificial Sequence Synthetic oligonucleotide
  modified_base (1)..(1): 2′-OMe-nucleotide
  modified_base (20)..(21): 2′-OMe-nucleotide
uagggucucaaaaggcuucuu

SEQ ID NO: 172 - RNA, 21 nt, Artificial Sequence
  Description of Artificial Sequence Synthetic oligonucleotide
  modified_base (1)..(1): 2′-deoxy-2′-fluoro-nucleotide
  modified_base (20)..(21): 2′-OMe-nucleotide
uagggucucaaaaggcuucuu

SEQ ID NO: 173 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-deoxy-nucleotide
  modified_base (20)..(21): 2′-OMe-nucleotide
uagggucucaaaaggcuucuu

SEQ ID NO: 174 - RNA, 21 nt, Artificial Sequence
  Description of Artificial Sequence Synthetic oligonucleotide
  modified_base (1)..(2): Phosphorothioate linkage
  modified_base (20)..(21): 2′-OMe-nucleotide
uagggucucaaaaggcuucuu

SEQ ID NO: 175 - RNA, 21 nt, Artificial Sequence
  Description of Artificial Sequence Synthetic oligonucleotide
  modified_base (1)..(1): 2′-deoxy-2′-fluoro-nucleotide
  modified_base (9)..(9): 2′-deoxy-2′-fluoro-nucleotide
  modified_base (17)..(17): 2′-deoxy-2′-fluoro-nucleotide
  modified_base (20)..(21): 2′-OMe-nucleotide
uagggucucaaaaggcuucuu

SEQ ID NO: 176 - RNA, 21 nt, Artificial Sequence
  Description of Artificial Sequence Synthetic oligonucleotide
  modified_base (1)..(1): 2′-deoxy-2′-fluoro-nucleotide
  modified_base (9)..(9): 2′-deoxy-2′-fluoro-nucleotide
  modified_base (11)..(12): 2′-deoxy-2′-fluoro-nucleotide
  modified_base (17)..(17): 2′-deoxy-2′-fluoro-nucleotide
  modified_base (20)..(21): 2′-OMe-nucleotide
uagggucucaaaaggcuucuu

SEQ ID NO: 177 - RNA, 21 nt, Artificial Sequence
  Description of Artificial Sequence Synthetic oligonucleotide
  modified_base (1)..(1): 2′-OMe-nucleotide
  modified_base (9)..(9): 2′-OMe-nucleotide
  modified_base (17)..(17): 2′-OMe-nucleotide
  modified_base (20)..(21): 2′-OMe-nucleotide
uagggucucaaaaggcuucuu

SEQ ID NO: 178 - RNA, 21 nt, Artificial Sequence
  Description of Artificial Sequence Synthetic oligonucleotide
  modified_base (1)..(1): 2′-deoxy-2′-fluoro-nucleotide
  modified_base (9)..(9): 2′-OMe-nucleotide
  modified_base (17)..(17): 2′-OMe-nucleotide
  modified_base (20)..(21): 2′-OMe-nucleotide
uagggucucaaaaggcuucuu

SEQ ID NO: 179 - RNA, 21 nt, Artificial Sequence
  Description of Artificial Sequence Synthetic oligonucleotide
  modified_base (20)..(21): 2′-OMe-nucleotide
uagggucucaaaaggcuucuu

SEQ ID NO: 180 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
uagggucucaaaaggcuucuu

SEQ ID NO: 181 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-OMe-nucleotide
  modified_base (6)..(6): 2′-deoxy-nucleotide
  modified_base (8)..(8): 2′-deoxy-nucleotide
  modified_base (9)..(9): 2′-OMe-nucleotide
  modified_base (11)..(12): 2′-OMe-nucleotide
  modified_base (18)..(18): 2′-OMe-nucleotide
  modified_base (20)..(21): 2′-OMe-nucleotide
uagggucucaaaaggcuucuu

SEQ ID NO: 182 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-OMe-nucleotide
  modified_base (6)..(6): 2′-deoxy-nucleotide
  modified_base (8)..(8): 2′-deoxy-nucleotide
  modified_base (9)..(9): 2′-OMe-nucleotide
  modified_base (11)..(12): 2′-OMe-nucleotide
  modified_base (18)..(18): 2′-OMe-nucleotide
  modified_base (20)..(21): 2′-OMe-nucleotide
uagggucucaaaaggcuucuu

SEQ ID NO: 183 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-OMe-nucleotide
  modified_base (6)..(6): 2′-deoxy-nucleotide
  modified_base (8)..(8): 2′-deoxy-nucleotide
  modified_base (9)..(9): 2′-OMe-nucleotide
  modified_base (11)..(12): 2′-OMe-nucleotide
  modified_base (17)..(18): 2′-OMe-nucleotide
  modified_base (20)..(21): 2′-OMe-nucleotide
uagggucucaaaaggcuucuu

SEQ ID NO: 184 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-deoxy-2′-fluoro-nucleotide
  modified_base (6)..(6): 2′-deoxy-nucleotide
  modified_base (8)..(8): 2′-deoxy-nucleotide
  modified_base (9)..(9): 2′-OMe-nucleotide
  modified_base (11)..(12): 2′-OMe-nucleotide
  modified_base (17)..(18): 2′-OMe-nucleotide
  modified_base (20)..(21): 2′-OMe-nucleotide
uagggucucaaaaggcuucuu

SEQ ID NO: 185 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
ccuuuugagacccugcugunn

SEQ ID NO: 186 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 187 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 188 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 189 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 190 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 191 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 192 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 193 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 194 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 195 - RNA, 21 nt, Artificial Sequence
  Description of Artificial Sequence Synthetic oligonucleotide
  modified_base (20)..(21): 2′-OMe-nucleotide
ccuuuugagacccugcuguuu

SEQ ID NO: 196 - RNA, 21 nt, Artificial Sequence
  Description of Artificial Sequence Synthetic oligonucleotide
  modified_base (1)..(1): 2′-OMe-nucleotide
  modified_base (3)..(3): 2′-OMe-nucleotide
  modified_base (5)..(5): 2′-OMe-nucleotide
  modified_base (7)..(7): 2′-OMe-nucleotide
  modified_base (9)..(9): 2′-OMe-nucleotide
  modified_base (20)..(21): 2′-OMe-nucleotide
ccuuuugagacccugcuguuu

SEQ ID NO: 197 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
acagcagggucucaaaaggnn

SEQ ID NO: 198 - RNA, 21 nt, Artificial Sequence
  Description of Artificial Sequence Synthetic oligonucleotide
  modified_base (20)..(21): 2′-OMe-nucleotide
acagcagggucucaaaagguu

SEQ ID NO: 199 - DNA, 21 bp, Artificial Sequence
  Description of Artificial Sequence Synthetic oligonucleotide
  Description of Combined DNA/RNA Molecule Synthetic oligonucleotide
  modified_base (1)..(8): 2′-deoxy-nucleotide
  modified_base (20)..(21): 2′-OMe-nucleotide
acagcagggucucaaaagguu

SEQ ID NO: 200 - DNA, 21 bp, Artificial Sequence
  Description of Artificial Sequence Synthetic oligonucleotide
  Description of Combined DNA/RNA Molecule Synthetic oligonucleotide
  modified_base (2)..(8): 2′-deoxy-nucleotide
  modified_base (20)..(21): 2′-OMe-nucleotide
acagcagggucucaaaagguu

SEQ ID NO: 201 - DNA, 21 bp, Artificial Sequence
  Description of Artificial Sequence Synthetic oligonucleotide
  Description of Combined DNA/RNA Molecule Synthetic oligonucleotide
  modified_base (3)..(8): 2′-deoxy-nucleotide
  modified_base (20)..(21): 2′-OMe-nucleotide
acagcagggucucaaaagguu

SEQ ID NO: 202 - DNA, 21 bp, Artificial Sequence
  Description of Artificial Sequence Synthetic oligonucleotide
  Description of Combined DNA/RNA Molecule Synthetic oligonucleotide
  modified_base (4)..(8): 2′-deoxy-nucleotide
  modified_base (20)..(21): 2′-OMe-nucleotide
acagcagggucucaaaagguu

SEQ ID NO: 203 - DNA, 21 bp, Artificial Sequence
  Description of Artificial Sequence Synthetic oligonucleotide
  Description of Combined DNA/RNA Molecule Synthetic oligonucleotide
  modified_base (5)..(8): 2′-deoxy-nucleotide
  modified_base (20)..(21): 2′-OMe-nucleotide
acagcagggucucaaaagguu

SEQ ID NO: 204 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-deoxy-nucleotide
  modified_base (3)..(3): 2′-deoxy-nucleotide
  modified_base (5)..(5): 2′-deoxy-nucleotide
  modified_base (7)..(7): 2′-deoxy-nucleotide
  modified_base (20)..(21): 2′-OMe-nucleotide
acagcagggucucaaaagguu

SEQ ID NO: 205 - DNA, 21 bp, Artificial Sequence
  Description of Artificial Sequence Synthetic oligonucleotide
  Description of Combined DNA/RNA Molecule Synthetic oligonucleotide
  modified_base (3)..(3): 2′-deoxy-nucleotide
  modified_base (5)..(5): 2′-deoxy-nucleotide
  modified_base (7)..(7): 2′-deoxy-nucleotide
  modified_base (20)..(21): 2′-OMe-nucleotide
acagcagggucucaaaagguu

SEQ ID NO: 206 - DNA, 21 bp, Artificial Sequence
  Description of Artificial Sequence Synthetic oligonucleotide
  Description of Combined DNA/RNA Molecule Synthetic oligonucleotide
  modified_base (2)..(2): 2′-deoxy-nucleotide
  modified_base (4)..(4): 2′-deoxy-nucleotide
  modified_base (6)..(6): 2′-deoxy-nucleotide
  modified_base (8)..(8): 2′-deoxy-nucleotide
  modified_base (20)..(21): 2′-OMe-nucleotide
acagcagggucucaaaagguu

SEQ ID NO: 207 - DNA, 21 bp, Artificial Sequence
  Description of Artificial Sequence Synthetic oligonucleotide
  Description of Combined DNA/RNA Molecule Synthetic oligonucleotide
  modified_base (4)..(4): 2′-deoxy-nucleotide
  modified_base (6)..(6): 2′-deoxy-nucleotide
  modified_base (8)..(8): 2′-deoxy-nucleotide
  modified_base (20)..(21): 2′-OMe-nucleotide
acagcagggucucaaaagguu

SEQ ID NO: 208 - DNA, 21 bp, Artificial Sequence
  Description of Artificial Sequence Synthetic oligonucleotide
  Description of Combined DNA/RNA Molecule Synthetic oligonucleotide
  modified_base (2)..(8): 2′-deoxy-nucleotide
  modified_base (14)..(14): 2′-OMe-nucleotide
  modified_base (16)..(16): 2′-OMe-nucleotide
  modified_base (18)..(18): 2′-OMe-nucleotide
  modified_base (20)..(21): 2′-OMe-nucleotide
acagcagggucucaaaagguu

SEQ ID NO: 209 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
ggaugacuaugugaaggcann

SEQ ID NO: 210 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 211 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 212 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 213 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 214 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 215 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 216 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 217 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 218 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 219 - RNA, 21 nt, Artificial Sequence
  Description of Artificial Sequence Synthetic oligonucleotide
  modified_base (20)..(21): 2′-OMe-nucleotide
ggaugacuaugugaaggcauu

SEQ ID NO: 220 - RNA, 21 nt, Artificial Sequence
  Description of Artificial Sequence Synthetic oligonucleotide
  modified_base (1)..(1): 2′-OMe-nucleotide
  modified_base (3)..(3): 2′-OMe-nucleotide
  modified_base (5)..(5): 2′-OMe-nucleotide
  modified_base (7)..(7): 2′-OMe-nucleotide
  modified_base (20)..(21): 2′-OMe-nucleotide
ggaugacuaugugaaggcauu

SEQ ID NO: 221 - RNA, 21 nt, Artificial Sequence
  Description of Artificial Sequence Synthetic oligonucleotide
  modified_base (11)..(11): 2′-deoxy-2′-fluoro-nucleotide
  modified_base (13)..(13): 2′-deoxy-2′-fluoro-nucleotide
  modified_base (20)..(21): 2′-OMe-nucleotide
ggaugacuaugugaaggcauu

SEQ ID NO: 222 - RNA, 21 nt, Artificial Sequence
  Description of Artificial Sequence Synthetic oligonucleotide
  modified_base (1)..(2): 2′-OMe-nucleotide
  modified_base (20)..(21): 2′-OMe-nucleotide
ggaugacuaugugaaggcauu

SEQ ID NO: 223 - RNA, 21 nt, Artificial Sequence
  Description of Artificial Sequence Synthetic oligonucleotide
  modified_base (1)..(2): 2′-OMe-nucleotide
  modified_base (20)..(21): 2′-OMe-nucleotide
ggaugacuaugugaaggcauu

SEQ ID NO: 224 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
ugccuucacauagucauccnn

SEQ ID NO: 225 - RNA, 21 nt, Artificial Sequence
  Description of Artificial Sequence Synthetic oligonucleotide
  modified_base (20)..(21): 2′-OMe-nucleotide
ugccuucacauagucauccuu

SEQ ID NO: 226 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-deoxy-nucleotide
  modified_base (5)..(6): 2′-deoxy-nucleotide
  modified_base (20)..(21): 2′-OMe-nucleotide
ugccuucacauagucauccuu

SEQ ID NO: 227 - DNA, 21 bp, Artificial Sequence
  Description of Artificial Sequence Synthetic oligonucleotide
  Description of Combined DNA/RNA Molecule Synthetic oligonucleotide
  modified_base (5)..(6): 2′-deoxy-nucleotide
  modified_base (20)..(21): 2′-OMe-nucleotide
ugccuucacauagucauccuu

SEQ ID NO: 228 - DNA, 21 bp, Artificial Sequence
  Description of Artificial Sequence Synthetic oligonucleotide
  Description of Combined DNA/RNA Molecule Synthetic oligonucleotide
  modified_base (5)..(6): 2′-deoxy-nucleotide
  modified_base (20)..(21): 2′-OMe-nucleotide
ugccuucacauagucauccuu

SEQ ID NO: 229 - DNA, 21 bp, Artificial Sequence
  Description of Artificial Sequence Synthetic oligonucleotide
  Description of Combined DNA/RNA Molecule Synthetic oligonucleotide
  modified_base (5)..(6): 2′-deoxy-nucleotide
  modified_base (20)..(21): 2′-OMe-nucleotide
ugccuucacauagucauccuu

SEQ ID NO: 230 - DNA, 21 bp, Artificial Sequence
  Description of Artificial Sequence Synthetic oligonucleotide
  Description of Combined DNA/RNA Molecule Synthetic oligonucleotide
  modified_base (5)..(6): 2′-deoxy-nucleotide
  modified_base (20)..(21): 2′-OMe-nucleotide
ugccuucacauagucauccuu

SEQ ID NO: 231 - DNA, 21 bp, Artificial Sequence
  Description of Artificial Sequence Synthetic oligonucleotide
  Description of Combined DNA/RNA Molecule Synthetic oligonucleotide
  modified_base (1)..(1): 2′-deoxy-nucleotide
  modified_base (5)..(5): 2′-deoxy-nucleotide
  modified_base (20)..(21): 2′-OMe-nucleotide
ugccuucacauagucauccuu

SEQ ID NO: 232 - DNA, 21 bp, Artificial Sequence
  Description of Artificial Sequence Synthetic oligonucleotide
  Description of Combined DNA/RNA Molecule Synthetic oligonucleotide
  modified_base (5)..(5): 2′-deoxy-nucleotide
  modified_base (20)..(21): 2′-OMe-nucleotide
ugccuucacauagucauccuu

SEQ ID NO: 233 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (20)..(21): 2′-OMe-nucleotide
ugccuucacauagucauccuu

SEQ ID NO: 234 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (20)..(21): 2′-OMe-nucleotide
ugccuucacauagucauccuu

SEQ ID NO: 235 - DNA, 21 bp, Artificial Sequence
  Description of Artificial Sequence Synthetic oligonucleotide
  Description of Combined DNA/RNA Molecule Synthetic oligonucleotide
  modified_base (5)..(6): 2′-deoxy-nucleotide
  modified_base (14)..(14): 2′-OMe-nucleotide
  modified_base (16)..(16): 2′-OMe-nucleotide
  modified_base (18)..(18): 2′-OMe-nucleotide
  modified_base (20)..(21): 2′-OMe-nucleotide
ugccuucacauagucauccuu

SEQ ID NO: 236 - RNA, 21 nt, Artificial Sequence
  Description of Artificial Sequence Synthetic oligonucleotide
  modified_base (4)..(4): 2′-deoxy-2′-fluoro-nucleotide
  modified_base (20)..(21): 2′-OMe-nucleotide
ugccuucacauagucauccuu

SEQ ID NO: 237 - RNA, 21 nt, Artificial Sequence
  Description of Artificial Sequence Synthetic oligonucleotide
  modified_base (9)..(9): 2′-OMe-nucleotide
  modified_base (11)..(11): 2′-OMe-nucleotide
  modified_base (13)..(13): 2′-OMe-nucleotide
  modified_base (15)..(15): 2′-OMe-nucleotide
  modified_base (17)..(17): 2′-OMe-nucleotide
  modified_base (19)..(21): 2′-OMe-nucleotide
ugccuucacauagucauccuu

SEQ ID NO: 238 - RNA, 21 nt, Artificial Sequence
  Description of Artificial Sequence Synthetic oligonucleotide
  modified_base (11)..(11): 2′-OMe-nucleotide
  modified_base (13)..(13): 2′-OMe-nucleotide
  modified_base (15)..(15): 2′-OMe-nucleotide
  modified_base (17)..(17): 2′-OMe-nucleotide
  modified_base (19)..(21): 2′-OMe-nucleotide
ugccuucacauagucauccuu

SEQ ID NO: 239 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
gaagccuuuugagacccugnn

SEQ ID NO: 240 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 241 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 242 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 243 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 244 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 245 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 246 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 247 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 248 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 249 - RNA, 21 nt, Artificial Sequence
  Description of Artificial Sequence Synthetic oligonucleotide
  modified_base (20)..(21): 2′-OMe-nucleotide
gaagccuuuugagacccuguu

SEQ ID NO: 250 - RNA, 21 nt, Artificial Sequence
  Description of Artificial Sequence Synthetic oligonucleotide
  modified_base (1)..(1): 2′-OMe-nucleotide
  modified_base (3)..(3): 2′-OMe-nucleotide
  modified_base (5)..(5): 2′-OMe-nucleotide
  modified_base (7)..(7): 2′-OMe-nucleotide
  modified_base (9)..(9): 2′-OMe-nucleotide
  modified_base (20)..(21): 2′-OMe-nucleotide
gaagccuuuugagacccuguu

SEQ ID NO: 251 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
cagggucucaaaaggcuucnn

SEQ ID NO: 252 - RNA, 21 nt, Artificial Sequence
  Description of Artificial Sequence Synthetic oligonucleotide
  modified_base (20)..(21): 2′-OMe-nucleotide
cagggucucaaaaggcuucuu

SEQ ID NO: 253 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
cagggucucaaaaggcuucuu

SEQ ID NO: 254 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
cagggucucaaaaggcuucuu

SEQ ID NO: 255 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
cagggucucaaaaggcuucuu

SEQ ID NO: 256 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
cagggucucaaaaggcuucuu

SEQ ID NO: 257 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
cagggucucaaaaggcuucuu

SEQ ID NO: 258 - DNA, 21 bp, Artificial Sequence
  Description of Artificial Sequence Synthetic oligonucleotide
  Description of Combined DNA/RNA Molecule Synthetic oligonucleotide
  modified_base (20)..(21): 2′-OMe-nucleotide
cagggucucaaaaggcuucuu

SEQ ID NO: 259 - DNA, 21 bp, Artificial Sequence
  Description of Artificial Sequence Synthetic oligonucleotide
  Description of Combined DNA/RNA Molecule Synthetic oligonucleotide
cagggucucaaaaggcuucuu

SEQ ID NO: 260 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
cagggucucaaaaggcuucuu

SEQ ID NO: 261 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (20)..(21): 2′-OMe-nucleotide
cagggucucaaaaggcuucuu

SEQ ID NO: 262 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (8)..(8): 2′-deoxy-nucleotide
  modified_base (14)..(14): 2′-OMe-nucleotide
  modified_base (16)..(16): 2′-OMe-nucleotide
  modified_base (18)..(18): 2′-OMe-nucleotide
  modified_base (20)..(21): 2′-OMe-nucleotide
cagggucucaaaaggcuucuu

SEQ ID NO: 263 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
ccucaucuacaccaacuaunn

SEQ ID NO: 264 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 265 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 266 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 267 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 268 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 269 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 270 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 271 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 272 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 273 - RNA, 21 nt, Artificial Sequence
  Description of Artificial Sequence Synthetic oligonucleotide
  modified_base (20)..(21): 2′-OMe-nucleotide
ccucaucuacaccaacuauuu

SEQ ID NO: 274 - RNA, 21 nt, Artificial Sequence
  Description of Artificial Sequence Synthetic oligonucleotide
  modified_base (1)..(1): 2′-OMe-nucleotide
  modified_base (3)..(3): 2′-OMe-nucleotide
  modified_base (5)..(5): 2′-OMe-nucleotide
  modified_base (7)..(7): 2′-OMe-nucleotide
  modified_base (9)..(9): 2′-OMe-nucleotide
  modified_base (20)..(21): 2′-OMe-nucleotide
ccucaucuacaccaacuauuu

SEQ ID NO: 275 - DNA, 21 bp, Artificial Sequence
  Description of Artificial Sequence Synthetic oligonucleotide
  Description of Combined DNA/RNA Molecule Synthetic oligonucleotide
  modified_base (20)..(21): a, c, t, g, u, unknown or other
auaguugguguagaugaggnn

SEQ ID NO: 276 - RNA, 21 nt, Artificial Sequence
  Description of Artificial Sequence Synthetic oligonucleotide
  modified_base (20)..(21): 2′-OMe-nucleotide
auaguugguguagaugagguu

SEQ ID NO: 277 - DNA, 21 bp, Artificial Sequence
  Description of Artificial Sequence Synthetic oligonucleotide
  Description of Combined DNA/RNA Molecule Synthetic oligonucleotide
  modified_base (2)..(2): 2′-deoxy-nucleotide
  modified_base (5)..(6): 2′-deoxy-nucleotide
  modified_base (20)..(21): 2′-OMe-nucleotide
auaguugguguagaugagguu

SEQ ID NO: 278 - DNA, 21 bp, Artificial Sequence
  Description of Artificial Sequence Synthetic oligonucleotide
  Description of Combined DNA/RNA Molecule Synthetic oligonucleotide
  modified_base (2)..(2): 2′-deoxy-nucleotide
  modified_base (5)..(6): 2′-deoxy-nucleotide
  modified_base (20)..(21): 2′-OMe-nucleotide
auaguugguguagaugagguu

SEQ ID NO: 279 - DNA, 21 bp, Artificial Sequence
  Description of Artificial Sequence Synthetic oligonucleotide
  Description of Combined DNA/RNA Molecule Synthetic oligonucleotide
  modified_base (5)..(6): 2′-deoxy-nucleotide
  modified_base (20)..(21): 2′-OMe-nucleotide
auaguugguguagaugagguu

SEQ ID NO: 280 - DNA, 21 bp, Artificial Sequence
  Description of Artificial Sequence Synthetic oligonucleotide
  Description of Combined DNA/RNA Molecule Synthetic oligonucleotide
  modified_base (5)..(6): 2′-deoxy-nucleotide
  modified_base (20)..(21): 2′-OMe-nucleotide
auaguugguguagaugagguu

SEQ ID NO: 281 - DNA, 21 bp, Artificial Sequence
  Description of Artificial Sequence Synthetic oligonucleotide
  Description of Combined DNA/RNA Molecule Synthetic oligonucleotide
  modified_base (5)..(6): 2′-deoxy-nucleotide
  modified_base (20)..(21): 2′-OMe-nucleotide
auaguugguguagaugagguu

SEQ ID NO: 282 - DNA, 21 bp, Artificial Sequence
  Description of Artificial Sequence Synthetic oligonucleotide
  Description of Combined DNA/RNA Molecule Synthetic oligonucleotide
  modified_base (5)..(5): 2′-deoxy-nucleotide
  modified_base (20)..(21): 2′-OMe-nucleotide
auaguugguguagaugagguu

SEQ ID NO: 283 - DNA, 21 bp, Artificial Sequence
  Description of Artificial Sequence Synthetic oligonucleotide
  Description of Combined DNA/RNA Molecule Synthetic oligonucleotide
  modified_base (5)..(5): 2′-deoxy-nucleotide
  modified_base (20)..(21): 2′-OMe-nucleotide
auaguugguguagaugagguu

SEQ ID NO: 284 - DNA, 21 bp, Artificial Sequence
  Description of Artificial Sequence Synthetic oligonucleotide
  Description of Combined DNA/RNA Molecule Synthetic oligonucleotide
  modified_base (2)..(2): 2′-deoxy-nucleotide
  modified_base (6)..(6): 2′-deoxy-nucleotide
  modified_base (20)..(21): 2′-OMe-nucleotide
auaguugguguagaugagguu

SEQ ID NO: 285 - DNA, 21 bp, Artificial Sequence
  Description of Artificial Sequence Synthetic oligonucleotide
  Description of Combined DNA/RNA Molecule Synthetic oligonucleotide
  modified_base (6)..(6): 2′-deoxy-nucleotide
  modified_base (20)..(21): 2′-OMe-nucleotide
auaguugguguagaugagguu

SEQ ID NO: 286 - DNA, 21 bp, Artificial Sequence
  Description of Artificial Sequence Synthetic oligonucleotide
  Description of Combined DNA/RNA Molecule Synthetic oligonucleotide
  modified_base (2)..(2): 2′-deoxy-nucleotide
  modified_base (5)..(6): 2′-deoxy-nucleotide
  modified_base (14)..(14): 2′-OMe-nucleotide
  modified_base (16)..(16): 2′-OMe-nucleotide
  modified_base (18)..(18): 2′-OMe-nucleotide
  modified_base (20)..(21): 2′-OMe-nucleotide
auaguugguguagaugagguu

SEQ ID NO: 287 - DNA, 986 bp, Homo sapiens
tgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccatgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagctaaaaaaaaaaaaaaaaaaaaaaaaaaaaa

SEQ ID NO: 288 - DNA, 50 bp, Artificial Sequence
  Description of Artificial Sequence Synthetic oligonucleotide
  Description of Combined DNA/RNA Molecule Synthetic oligonucleotide
ctcgaggggcaactgaagccttttgagaccctgctgtcccaggcggccgc

SEQ ID NO: 289 - DNA, 50 bp, Artificial Sequence
  Description of Artificial Sequence Synthetic oligonucleotide
  Description of Combined DNA/RNA Molecule Synthetic oligonucleotide
ctcgagctgggacagcagggtctcaaaaggcttcagttgcccgcggccgc

SEQ ID NO: 290 - DNA, 50 bp, Artificial Sequence
  Description of Artificial Sequence Synthetic oligonucleotide
  Description of Combined DNA/RNA Molecule Synthetic oligonucleotide
ctcgaggggcaactctacgcaaaacagaccctgctgtcccaggcggccgc

SEQ ID NO: 291 - DNA, 71 bp, Artificial Sequence
  Description of Artificial Sequence Synthetic oligonucleotide
  Description of Combined DNA/RNA Molecule Synthetic oligonucleotide
ctcgaggggcaactctacgcaaaacagaccctgctctacgcaaaacagaccctgctgtcccaggcggccgc

SEQ ID NO: 292 - DNA, 92 bp, Artificial Sequence
  Description of Artificial Sequence Synthetic oligonucleotide
  Description of Combined DNA/RNA Molecule Synthetic oligonucleotide
ctcgaggggcaactctacgcaaaacagaccctgctctacgcaaaacagaccctgctctacgcaaaacagaccctgctgtcccaggcggccgc

SEQ ID NO: 293 - DNA, 113 bp, Artificial Sequence
  Description of Artificial Sequence Synthetic oligonucleotide
  Description of Combined DNA/RNA Molecule Synthetic oligonucleotide
ctcgaggggcaactctacgcaaaacagaccctgctctacgcaaaacagaccctgctctacgcaaaacagaccctgctctacgcaaaacagaccctgctgtcccaggcggccgc

SEQ ID NO: 294 - RNA, 21 nt, Artificial Sequence
  Description of Artificial Sequence Synthetic oligonucleotide
  modified_base (1)..(3): 2′-OMe-nucleotide
  modified_base (6)..(6): 2′-OMe-nucleotide
  modified_base (11)..(11): 2′-OMe-nucleotide
  modified_base (13)..(14): 2′-OMe-nucleotide
  modified_base (17)..(17): 2′-OMe-nucleotide
  modified_base (20)..(21): 2′-OMe-nucleotide
ccuuuugagacccugcuguuu

SEQ ID NO: 295 - RNA, 21 nt, Artificial Sequence
  Description of Artificial Sequence Synthetic oligonucleotide
  modified_base (1)..(3): 2′-OMe-nucleotide
  modified_base (20)..(21): 2′-OMe-nucleotide
ccuuuugagacccugcuguuu

SEQ ID NO: 296 - RNA, 21 nt, Artificial Sequence
  Description of Artificial Sequence Synthetic oligonucleotide
  modified_base (1)..(3): 2′-OMe-nucleotide
  modified_base (6)..(6): 2′-OMe-nucleotide
  modified_base (11)..(11): 2′-OMe-nucleotide
  modified_base (13)..(14): 2′-OMe-nucleotide
  modified_base (17)..(17): 2′-OMe-nucleotide
  modified_base (20)..(21): 2′-OMe-nucleotide
ccuuuugagacccugcuguuu

SEQ ID NO: 297 - RNA, 21 nt, Artificial Sequence
  Description of Artificial Sequence Synthetic oligonucleotide
  modified_base (1)..(3): 2′-OMe-nucleotide
  modified_base (6)..(6): 2′-OMe-nucleotide
  modified_base (11)..(11): 2′-OMe-nucleotide
  modified_base (13)..(14): 2′-OMe-nucleotide
  modified_base (17)..(17): 2′-OMe-nucleotide
  modified_base (20)..(21): 2′-OMe-nucleotide
ccuuuugagacccugcuguuu

SEQ ID NO: 298 - DNA, 21 bp, Artificial Sequence
  Description of Artificial Sequence Synthetic oligonucleotide
  Description of Combined DNA/RNA Molecule Synthetic oligonucleotide
  modified_base (4)..(4): 2′-deoxy-nucleotide
  modified_base (6)..(6): 2′-deoxy-nucleotide
  modified_base (8)..(8): 2′-deoxy-nucleotide
  modified_base (20)..(21): 2′-OMe-nucleotide
acagcagggucucaaaagguu

SEQ ID NO: 299 - DNA, 21 bp, Artificial Sequence
  Description of Artificial Sequence Synthetic oligonucleotide
  Description of Combined DNA/RNA Molecule Synthetic oligonucleotide
  modified_base (2)..(2): 2′-OMe-nucleotide
  modified_base (4)..(4): 2′-deoxy-nucleotide
  modified_base (5)..(5): 2′-OMe-nucleotide
  modified_base (6)..(6): 2′-deoxy-nucleotide
  modified_base (8)..(8): 2′-deoxy-nucleotide
  modified_base (13)..(13): 2′-OMe-nucleotide
  modified_base (20)..(21): 2′-OMe-nucleotide
acagcagggucucaaaagguu

SEQ ID NO: 300 - DNA, 21 bp, Artificial Sequence
  Description of Artificial Sequence Synthetic oligonucleotide
  Description of Combined DNA/RNA Molecule Synthetic oligonucleotide
  modified_base (2)..(2): 2′-OMe-nucleotide
  modified_base (4)..(4): 2′-deoxy-nucleotide
  modified_base (5)..(5): 2′-OMe-nucleotide
  modified_base (6)..(6): 2′-deoxy-nucleotide
  modified_base (8)..(8): 2′-deoxy-nucleotide
  modified_base (13)..(13): 2′-OMe-nucleotide
  modified_base (20)..(21): 2′-OMe-nucleotide
acagcagggucucaaaagguu

SEQ ID NO: 301 - DNA, 21 bp, Artificial Sequence
  Description of Artificial Sequence Synthetic oligonucleotide
  Description of Combined DNA/RNA Molecule Synthetic oligonucleotide
  modified_base (2)..(2): 2′-OMe-nucleotide
  modified_base (4)..(4): 2′-deoxy-nucleotide
  modified_base (5)..(5): 2′-OMe-nucleotide
  modified_base (6)..(6): 2′-deoxy-nucleotide
  modified_base (8)..(8): 2′-deoxy-nucleotide
  modified_base (11)..(11): 2′-OMe-nucleotide
  modified_base (13)..(13): 2′-OMe-nucleotide
  modified_base (15)..(15): 2′-OMe-nucleotide
  modified_base (17)..(17): 2′-OMe-nucleotide
  modified_base (19)..(21): 2′-OMe-nucleotide
acagcagggucucaaaagguu

SEQ ID NO: 302 - RNA, 21 nt, Artificial Sequence
  Description of Artificial Sequence Synthetic oligonucleotide
  modified_base (1)..(2): 2′-OMe-nucleotide
  modified_base (11)..(11): 2′-OMe-nucleotide
  modified_base (13)..(13): 2′-OMe-nucleotide
  modified_base (20)..(21): 2′-OMe-nucleotide
ggaugacuaugugaaggcauu

SEQ ID NO: 303 - RNA, 21 nt, Artificial Sequence
  Description of Artificial Sequence Synthetic oligonucleotide
  modified_base (1)..(2): 2′-OMe-nucleotide
  modified_base (4)..(4): 2′-OMe-nucleotide
  modified_base (8)..(8): 2′-OMe-nucleotide
  modified_base (10)..(10): 2′-OMe-nucleotide
  modified_base (12)..(12): 2′-OMe-nucleotide
  modified_base (18)..(18): 2′-OMe-nucleotide
  modified_base (20)..(21): 2′-OMe-nucleotide
ggaugacuaugugaaggcauu

SEQ ID NO: 304 - DNA, 21 bp, Artificial Sequence
  Description of Artificial Sequence Synthetic oligonucleotide
  Description of Combined DNA/RNA Molecule Synthetic oligonucleotide
  modified_base (4)..(4): 2′-deoxy-nucleotide
  modified_base (6)..(6): 2′-deoxy-nucleotide
  modified_base (8)..(8): 2′-deoxy-nucleotide
  modified_base (20)..(21): 2′-OMe-nucleotide
ugccuucacauagucauccuu

SEQ ID NO: 305 - DNA, 21 bp, Artificial Sequence
  Description of Artificial Sequence Synthetic oligonucleotide
  Description of Combined DNA/RNA Molecule Synthetic oligonucleotide
  modified_base (4)..(4): 2′-deoxy-nucleotide
  modified_base (6)..(6): 2′-deoxy-nucleotide
  modified_base (8)..(8): 2′-deoxy-nucleotide
  modified_base (20)..(21): 2′-OMe-nucleotide
ugccuucacauagucauccuu

(19) United States
(12) Patent Application Publication
(10) Pub. No.: US 2022/0275373 A1
(43) Pub. Date: Sep. 1, 2022

(54) METHODS AND COMPOSITIONS FOR TREATING MALIGNANT TUMORS ASSOCIATED WITH KRAS MUTATION

(71) Applicant: Nitto Denko Corporation, Osaka (JP)

(72) Inventors: Kenjirou Minomi, Osaka (JP); Jihua Liu, San Marcos, CA (US); Li Wang, San Diego, CA (US); Bharat Majeti, San Diego, CA (US); Roger Adami, Carlsbad, CA (US); Wenbin Ying, San Diego, CA (US)

(21) Appl. No.: 17/664,032

(22) Filed: May 18, 2022

Related U.S. Application Data
(63) Continuation of application No. 16/540,973, filed on Aug. 14, 2019, now Pat. No. 11,352,628.
Application No. 16/540,973 is a division of application No. 15/636,528, filed on Jun. 28, 2017, now abandoned.
Application No. 15/636,528 is a continuation-in-part of application No. 15/434,318, filed on Feb. 16, 2017, now Pat. No. 10,792,299.
Application No. 15/434,318 is a continuation-in-part of application No. 14/979,573, filed on Dec. 28, 2015, now Pat. No. 9,580,710.
(60) Provisional application No. 62/266,672, filed on Dec. 13, 2015.
(60) Provisional application No. 62/184,204, filed on Jun. 24, 2015.

(30) Foreign Application Priority Data
Dec. 26, 2014 (JP) 2014-266198

(51) Int. Cl. C12N 15/113; A61K 9/127; A61K 31/713; A61K 31/7105

(52) U.S. Cl. CPC C12N 15/1137; A61K 9/127; A61K 31/713; C12Y 205/01018; A61K 31/7105; C12N 15/1135; C12N 15/113; C12N 2310/321; C12N 2310/14; C12N 2320/32; C12N 2310/322; C12N 2310/344; C12N 2310/531

(57) ABSTRACT

This invention provides methods and compositions for preventing, treating or ameliorating one or more symptoms of a malignant tumor, which may be associated with KRAS mutation in a mammal in need thereof, by administering to the mammal a therapeutically effective amount of a composition comprising one or more RNAi molecules that are active in reducing expression of GST-π.

### SEQUENCE LISTING

This application includes a Sequence Listing submitted electronically as an ASCII file created on Jan. 2, 2016, named ND7023946US_SL.txt, which is 120,619 bytes in size, and is hereby incorporated by reference in its entirety.

### BACKGROUND OF THE INVENTION

Glutathione S-transferases (IUBMB EC 2.5.1.18) are a family of enzymes that play an important role in detoxification by catalyzing the conjugation of many hydrophobic and electrophilic compounds with reduced glutathione. Based on their biochemical, immunologic, and structural properties, the soluble GSTs are categorized into four main classes: alpha, mu, pi, and theta. Some of these forms are suggested to act to prevent carcinogenesis by detoxifying proximate or ultimate carcinogens, especially electrophilic agents including Michael reaction acceptors, diphenols, quinones, isothiocyanates, peroxides, vicinal dimercaptans, etc. However, in neoplastic cells, specific forms are known to be expressed and have been known to participate in their resistance to anticancer drugs.

The glutathione S-transferase-π gene (GSTP1) is a polymorphic gene encoding active, functionally different GSTP1 variant proteins that are thought to function in xenobiotic metabolism and play a role in susceptibility to cancer. It is expressed abundantly in tumor cells. See, e.g., Aliya S. et al. Mol Cell Biochem., 2003 November; 253(1-2):319-327. Glutathione S-transferase-P is an enzyme that in humans is encoded by the GSTP1 gene. See, e.g., Bora P S, et al. (October 1991) J. Biol. Chem., 266 (25): 16774-16777. The GST-π isoenzyme has been shown to catalyze the conjugation of GSH with some alkylating anti-cancer agents, suggesting that over-expression of GST-n would result in tumor cell resistance.

Elevated serum GST-π levels were observed in patients with various gastrointestinal malignancies including gastric, esophageal, colonic, pancreatic, hepatocellular, and biliary tract cancers. Patients with benign gastrointestinal diseases had normal GST-π, but some patients with chronic hepatitis and cirrhosis had slightly elevated levels. Over 80% of patients with Stage III or IV gastric cancer and even about 50% of those with Stage I and II had elevated serum GST-π. See, e.g., Niitsu Y, et al. Cancer, 1989 Jan. 15; 63(2)317-23. Elevated GST-π levels in plasma were observed in patients with oral cancer, but patients with benign oral diseases had normal GST-π levels. GST-π was found to be a useful marker for evaluating the response to chemotherapy, for monitoring postoperative tumor resectability or tumor burden, and for predicting the recurrence of tumor in patients with oral cancer. See, e.g., Hirata S. et al. Cancer, 1992 Nov. 15:70(10):2381-7.

Immunohistochemical studies have revealed that many cancers, histologically classified as adenocarcinomas or squamous cell carcinomas, express GST-π. Plasma or serum GST-π levels are increased in 30-50% of patients with cancers of the gastrointestinal tract. This form is also suggested to participate in resistance to anticancer drugs such as cisplatin and daunorubicin, and its expression in cancer tissues may be of prognostic value in cancer patients.

The protein product of the normal human KRAS gene (V-Ki-ras2 Kirsten rat sarcoma viral oncogene homolog) performs a signaling function in normal tissue, and the mutation of a KRAS gene is a putative step in the development of many cancers. See, e g. Kranenburg O, November 2005, Biochim. Biophys. Acta, 1756(481-82. The KRAS protein is a GTPase and is involved in several signal transduction pathways. KRAS acts as a molecular on/off switch which activates proteins necessary for the propagation of growth factor and signals of other receptors such as c-Raf and PI 3-kinase.

Mutation in KRAS can be related to malignant tumors, such as lung adenocarcinoma, mucinous adenoma, ductal carcinoma of the pancreas, and colorectal carcinoma. In human colorectal cancer, KRAS mutation appears to induce overexpression of GST-π via activation of AP-1. See, e.g., Miyanishi et al., Gastroenterology, 2001; 121 (4):865-74.

Mutant KRAS is found in colon cancer (Burmer G C, Loeb L A, 1989, Proc. Natl. Acad, Sci. U.S.A., 86(7):2403-2407), pancreatic cancer (Almoguera C, et al., 1988, Cell, 53(4):549-554) and lung cancer (Tam I Y et al., 2006, Clin. Cancer Res., 12(5):1647-1653). KRAS accounts for 90% of RAS mutations in lung adenocarcinomas (Forbes S, et al. Cosmic 2005. Br J Cancer, 2006; 94:318-322).

KRAS gene may also be amplified in colorectal cancer. KRAS amplification can be mutually exclusive with KRAS mutations. See, e.g., Valtorta E, et al., 2013, Int. J. Cancer, 133(5):1259-65. Amplification of wild-type KRAS also has been observed in ovarian, gastric, uterine, and lung cancers. See, e.g., Chen Y, et al., 2014, PLoS ONE, 9(5):e98293.

Expression of GST-π increases in various cancer cells, which may be related to resistance to some anticancer agents. See, e.g. Ban et al., Cancer Res., 1996, 56(15):3577-82; Nakajima et al., J Pharmacol Exp Ther., 2003, 306(3):861-9.

Agents for suppressing GST-π have been disclosed for inducing apoptosis in cells. However, such compositions and techniques also caused autophagy and required the combined action of various agents. See, e.g., US 2014/0315975 A1. Moreover, suppressing GST-π has not been found to shrink or reduce tumors. For example, in a cancer that was overexpressing GST-π, the weights of tumors were not affected by suppressing GST-π, although other effects were observed. See, e.g., Hokaiwado et al., Carcinogenesis, 2008, 29(6):1134-1138.

There is an urgent need for methods and compositions to develop therapies for patients with KRAS associated malignancies.

What is needed are methods and compositions for preventing or treating malignant tumors. There is a continuing need for RNAi molecules, and other structures and compositions for preventing, treating, reducing or shrinking malignant tumors.

### BRIEF SUMMARY

This invention relates to the fields of biopharmaceuticals and therapeutics composed of nucleic acid based molecules. More particularly, this invention relates to tumor therapies for preventing, treating or ameliorating KRAS-associated cancers in which the cancer cells contain a KRAS mutation or display aberrant KRAS expression levels. This invention further relates to a pharmaceutical composition containing one or more RNAi molecules for inhibiting expression of GST-π.

This invention relates to the surprising discovery that malignant tumor size can be reduced in vivo by treatment with siRNA inhibitors of GST-π.

In some embodiments, malignant tumors containing a KRAS mutation or displaying aberrant KRAS expression levels can be reduced by treatment with siRNA agents that modulate expression of GST-π.

This invention relates to methods and compositions for nucleic acid based therapeutic compounds against malignant tumors. In some embodiments, this invention provides RNAi molecules, structures and compositions that can silence expression of GST-π. The structures and compositions of this disclosure can be used in preventing, treating or reducing the size of malignant tumors.

This invention provides compositions and methods that may be used for treating a neoplasia in a subject. In particular, this invention provides therapeutic compositions that can decrease the expression of a GST-π nucleic acid molecule or polypeptide for treating a KRAS-associated neoplasia without unwanted autophagy.

In some aspects, this invention includes an inhibitory nucleic acid molecule that corresponds to, or is complementary to at least a fragment of a GST-π nucleic acid molecule, and that decreases GST-π expression in a cell.

In further aspects, the invention features a double-stranded inhibitory nucleic acid molecule that corresponds to, or is complementary to at least a fragment of a GST-π nucleic acid molecule that decreases GST-π expression in a cell. In certain embodiments, the double-stranded nucleic acid molecule is a siRNA or a shRNA.

In some aspects, this invention includes a vector encoding an inhibitory nucleic acid molecule described above. A vector can be a retroviral, adenoviral, adeno-associated viral, or lentiviral vector. In further embodiments, a vector can contain a promoter suitable for expression in a mammalian cell. Additional embodiments include cancer cells containing a KRAS mutation or displaying aberrant KRAS expression levels, which can also contain the vector, or an inhibitory nucleic acid molecule of any one of the above aspects. In further embodiments, the cells can be neoplastic cells in vivo.

In some embodiments, this invention includes methods for decreasing GST-π expression in a malignant tumor cell containing a KRAS mutation or displaying aberrant KRAS expression. Methods can include contacting the cell with an effective amount of an inhibitory nucleic acid molecule corresponding to, or complementary to at least a portion of a GST-π nucleic acid molecule, where the inhibitory nucleic acid molecule inhibits expression of a GST-π polypeptide, thereby decreasing GST-π expression in the cell.

In certain embodiments, the inhibitory nucleic acid molecule can be an antisense nucleic acid molecule, a small interfering RNA (siRNA), or a double-stranded RNA (dsRNA) that is active for inhibiting gene expression.

In additional embodiments, methods of this invention can decrease GST-π transcription or translation in malignant tumors.

In particular embodiments, this invention includes methods for decreasing GST-π expression in a malignant tumor cell, where the cell can be a human cell, a neoplastic cell, a cell in vivo, or a cell in vitro.

Embodiments of this invention can also provide methods for treating a subject having a neoplasm, where neoplasm cancer cells contain a KRAS mutation or display aberrant KRAS expression levels. Methods can involve administering to the subject an effective amount of an inhibitory nucleic acid molecule corresponding to, or complementary to a GST-π nucleic acid molecule, where the inhibitory nucleic acid molecule reduces GST-π expression, thereby treating the neoplasm. In some embodiments, methods of this invention can decrease the size of a neoplasm, relative to the size of the neoplasm prior to treatment or without treatment.

In various embodiments, an inhibitory nucleic acid molecule can be delivered in a liposome, a polymer, a microsphere, a nanoparticle, a gene therapy vector, or a naked DNA vector.

In further aspects, this invention features methods for treating a subject, e.g. a human patient, having a neoplasm in which the neoplasm cancer cells contain a KRAS mutation or display aberrant KRAS expression levels. In certain embodiments, the methods can include administering to the subject an effective amount of an inhibitory nucleic acid molecule, where the inhibitory nucleic acid molecule is an antisense nucleic acid molecule, a siRNA, or a dsRNA that inhibits expression of a GST-π polypeptide.

In particular embodiments, a cell of the neoplasm overexpresses GST-π.

In certain embodiments, the neoplasm can be a malignant tumor, or lung cancer, or pancreatic cancer.

Embodiments of this invention include the following:

A pharmaceutical composition for the treatment or therapy of a tumor associated with a mutation in the KRAS gene or overexpression of wild-type KRAS gene, the composition comprising RNAi molecules and pharmaceutically acceptable excipients, wherein the RNAi molecules comprise a nucleotide sequence corresponding to a target sequence of GST-π.

In some embodiments, the pharmaceutical composition includes RNAi molecules that have a duplex region comprising a nucleotide sequence corresponding to a target sequence of GST-πmRNA.

In certain aspects, the RNAi molecules are siRNAs or shRNAs that are active for suppressing gene expression.

The pharmaceutical composition can include pharmaceutically acceptable excipients such as one or more lipid compounds. The lipid compounds may include lipid nanoparticles. In certain embodiments, the lipid nanoparticles can encapsulate the RNAi molecules.

This invention further contemplates methods for preventing, treating or ameliorating one or more symptoms of a malignant tumor associated with KRAS mutation in a mammal in need thereof, the method comprising:

identifying a tumor cell in the mammal, the tumor cell comprising at least one of: (i) a mutation of the KRAS gene, and (ii) an aberrant expression level of KRAS protein; and

administering to the mammal a therapeutically effective amount of a composition comprising one or more RNAi molecules that are active in reducing expression of GST-π.

In such methods, the mammal can be a human, and the GST-π can be a human GST-π. The RNAi molecule can be a siRNA, shRNA, or microRNA.

In certain embodiments, the RNAi molecule can have a duplex region, wherein the duplex region can include a nucleotide sequence corresponding to a target sequence of GST-π mRNA. The RNAi molecule can decrease expression of GST-π in the mammal.

In some embodiments, the administration can decrease expression of GST-π in the mammal by at least 5% for at least 5 days. In certain embodiments, the administration can decrease the volume of the malignant tumor in the mammal by at least 5%, or at least 10%, or at least 20%, or at least 30%, or at least 40%, or at least 50%. In additional embodiments, the method can reduce one or more symptoms of the malignant tumor, or delay or terminate progression or growth of the malignant tumor.

In certain embodiments, the administration can reduce growth of malignant tumor cells in the subject. The administration can reduce growth for at least 2%, or at least 5%, or at least 10%, or at least 15%, or at least 20% of the malignant tumor cells in the subject.

In general, the tumor cells can have increased levels of expression of wild type KRAS protein compared to that in a normal cell. In some embodiments, the tumor cell over-express wild-type GST-πRNA or protein.

In particular, the tumor cell can have mutations in the KRAS protein at one or more of residues 12, 13 and 61.

This invention contemplates that the tumor cell can have mutations in the KRAS protein, and the tumor can be a cancer selected from lung cancer, colon cancer, and pancreatic cancer.

In some embodiments, the tumor cell can have mutations in the KRAS protein, and the tumor can be a sarcoma selected from the group consisting of lung adenocarcinoma, mucinous adenoma, ductal carcinoma of the pancreas, and colorectal carcinoma. In certain embodiments, the malignant tumor can be a sarcoma selected from the group of lung adenocarcinoma, mucinous adenoma, ductal carcinoma of the pancreas, colorectal carcinoma, breast cancer, and fibrosarcoma. Also, the malignant tumor can be located in an anatomical region selected from the group of lung, colon, pancreas, gallbladder, liver, breast, and any combination thereof.

Aspects of this invention can provide methods in which the administration is performed from 1 to 12 times per day. The administration can be performed for a duration of 1, 2, 3, 4, 5, 6 or 7 days. In certain embodiments, the administration can be performed for a duration of 1, 2, 3, 4, 5, 6, 8, 10 or 12 weeks.

A dose for administration can be from 0.01 to 2 mg/kg of the RNAi molecules at least once per day for a period up to twelve weeks. In some embodiments, the administration can provide a mean AUC(0-last) of from 1 to 1000 ug*min/mL and a mean Cₘₐₓ of from 0.1 to 50 ug/mL for the GST-πRNAi molecule.

The administration can be by intravenous injection, intradermal injection, subcutaneous injection, intramuscular injection, intraperitoneal injection, oral, topical, infusion, or inhaled.

These and other aspects will become apparent from the following description of the embodiments taken in conjunction with the following drawings, although variations and modifications therein may be affected without departing from the spirit and scope of the novel concepts of the disclosure.

### BRIEF DESCRIPTION OF THE DRAWINGS

FIG. 1: shows the profound reduction of orthotopic lung cancer tumors in vivo by a siRNA of this invention targeted to GST-π. The GST-π siRNA was administered in a liposomal formulation at a dose of 2 mg/kg to athymic nude mice presenting A549 orthotopic lung cancer tumors. Final primary tumor weights were measured at necropsy for the treatment group and a vehicle control group. The GST-π siRNA showed significant efficacy for inhibition of lung cancer tumors in this six-week study. As shown in FIG. 1, after 43 days, the GST-π siRNA showed markedly advantageous tumor inhibition, with final primary tumor average weights significantly reduced by 2.8-fold, as compared to control.

FIG. 2: shows tumor inhibition efficacy in vivo for a GST-π siRNA. A cancer xenograft model using A549 cells was utilized with a relatively low dose of siRNA at 0.75 mg/kg. The GST-π siRNA showed advantageous tumor inhibition within a few days. After 36 days, the GST-π siRNA showed markedly advantageous tumor inhibition, with final tumor average volumes significantly reduced by about 2-fold, as compared to control.

FIG. 3: shows tumor inhibition efficacy in vivo for a GST-π siRNA at the endpoint of FIG. 2. The GST-π siRNA showed advantageous tumor inhibition with average tumor weights reduced by more than 2-fold.

FIG. 4: shows that a GST-π siRNA of this invention greatly increased cancer cell death by apoptosis in vitro. The GST-π siRNA caused upregulation of PUMA, a biomarker for apoptosis, which is associated with loss in cell viability. In FIG. 4, the expression of PUMA was greatly increased from 2-6 days after transfection of the GST-π siRNA.

FIG. 5: shows that a GST-π siRNA of this invention provided knockdown efficacy for A549 xenograft tumors in vivo. Dose dependent knockdown of GST-π mRNA was observed in athymic nude (nu/nu) female mice (Charles River) with the siRNA targeted to GST-π. As shown in FIG. 5, at a dose of 4 mg/kg, significant reduction of about 40% in GST-π mRNA was detected 24 hours after injection.

FIG. 6: shows that a GST-π siRNA of this invention inhibited pancreatic cancer xenograft tumors in vivo. The GST-π siRNA provided gene silencing potency in vivo when administered in a liposomal formulation to pancreatic cancer xenograft tumors in athymic nude female mice, 6 to 8 weeks old. As shown in FIG. 6, a dose response was obtained with doses ranging from 0.375 mg/kg to 3 mg/kg of siRNA targeted to GST-π. The GST-π siRNA showed advantageous tumor inhibition within a few days after administration, the tumor volume being reduced by about 2-fold at the endpoint.

FIG. 7: shows that a GST-π siRNA of this invention exhibited increased serum stability. As shown in FIG. 7, the half-life (t_{1/2}) in serum for both the sense strand (FIG. 7, top) and antisense strand (FIG. 7, bottom) of a GST-π siRNA was about 100 minutes.

FIG. 8: shows that a GST-π siRNA of this invention exhibited enhanced stability in formulation in plasma. FIG. 8 shows incubation of a liposomal formulation of a GST-π siRNA in 50% human serum in PBS, and detection of remaining siRNA at various time points. As shown in FIG. 8, the half-life (t_{1/2}) in plasma of the formulation of the GST-π siRNA was significantly longer than 100 hours.

FIG. 9: shows in vitro knockdown for the guide strand of a GST-πsiRNA. As shown in FIG. 9, the guide strand knockdown of the GST-π siRNA was approximately exponential, as compared to a control with scrambled sequence that exhibited no effect.

FIG. 10: shows in vitro knockdown for the passenger strand of the GST-π siRNA of FIG. 9. As shown in FIG. 10, the passenger strand off target knockdown for the GST-π siRNA was greatly reduced, with essentially no effect.

FIG. 11: shows in vitro knockdown for the guide strands of several highly active GST-π siRNAs. As shown in FIG. 11, the guide strand knockdown activities of the GST-π siRNAs were approximately exponential.

FIG. 12: shows in vitro knockdown for the passenger strand of the GST-π siRNAs of FIG. 11. As shown in FIG. 12, the passenger strand off target knockdown activities for the GST-π siRNAs were significantly reduced below about 500 pM.

FIG. 13: shows in vitro knockdown for the guide strand of a highly active GST-π siRNA. As shown in FIG. 13, the guide strand knockdown activity of the GST-π siRNA was approximately exponential.

FIG. 14: shows in vitro knockdown for the passenger strand of the GST-π siRNA of FIG. 13. As shown in FIG. 14, the passenger strand off target knockdown activity for the GST-π siRNA was significantly reduced.

FIG. 15: shows tumor inhibition efficacy in vivo for GST-π siRNAs having structure based on siRNA A9. A cancer xenograft model using A549 cells was utilized with a relatively low dose of siRNA at 0.5 mg/kg. The GST-π siRNAs showed advantageous tumor inhibition within a few days. After 36 days, the GST-π siRNAs showed markedly advantageous tumor inhibition, with final tumor average volumes significantly reduced by about 2-fold, as compared to control.

FIG. 16: shows tumor inhibition efficacy in vivo for a GST-π siRNA having structure based on siRNA B13. A cancer xenograft model using A549 cells was utilized with a relatively low dose of siRNA at 0.75 mg/kg. The GST-π siRNA showed advantageous tumor inhibition within a few days. After 36 days, the GST-π siRNA showed markedly advantageous tumor inhibition, with final tumor average volumes significantly reduced by about 2-fold, as compared to control.

### DETAILED DESCRIPTION OF THE INVENTION

The invention provides methods for utilizing therapeutic compositions that decrease the expression of a GST-π nucleic acid molecule or polypeptide for the treatment of a neoplasia in a subject, wherein the neoplasia is associated with cells containing a KRAS mutation or displaying aberrant KRAS expression levels.

The therapeutic compositions of this invention can include inhibitory nucleic acid molecules such as siRNAs, shRNAs, and antisense RNAs.

GST-π denotes an enzyme, which is encoded by the GSTP1 gene, and catalyzes glutathione conjugation. GST-π is present in various animals, including humans, and its sequence information is known and given in NCBI database accession numbers (e.g., human: NP_000843 (NM_000852), rat: NP_036709 (NM_012577), mouse: NP_038569 (NM_013541), etc.

By “GST-π polypeptide” is meant a protein or protein variant, or fragment thereof, that is substantially identical to at least a portion of a protein encoded by the GST-π coding sequence. By “GST-π, nucleic acid molecule” is meant a polynucleotide encoding a GST-π polypeptide or variant, or fragment thereof.

Occurrence of a mutation of a gene sequence or an amino acid sequence between biological individuals may not impair the physiological function of a protein. GST-π and GSTP1 gene in this invention are not limited to a protein or nucleic acid having the same sequence as the GST-π sequences listed herein, and can include those that have a sequence that is different from the above sequence by one or more amino acids or bases, for example, one, two, three, four, five, six, seven, eight, nine, or ten amino acids or bases, but have an equivalent function to that of the known GST-π.

The sequence of Human glutathione S-transferase gene (GST-π), complete CDS, GenBank Accession No.: U12472, is shown in Table 1.

TABLE 1
The complete seguence of the human GSTπ gene. (SEQ ID NO: 1)

| | |
|---|---|
| 1 | gtggctcacc tgtacccagc acttgggaag ccgaggcgtg cagatcacct aagtcaggag |
| 61 | ttcgagacca gcccggccaa catggtgaaa ccccgtctct actaaaaata caaaaatcag |
| 121 | ccagatgtgg cacgcaccta tatccaccta ctcgggaggc tgaagcagaa tgcttaaccc |
| 181 | gagaggcgga ggttgcagtg agccgcccag atcgcgccac tgcactccag cctgggccac |
| 241 | agcgtgagac tactcataaa ataaaataaa ataaaataaa ataaaataaa ataaaataaa |
| 301 | ataataaaat aaaataaaat aaaataaaat ataaaataaa ataaaataaa ataaaataaa |
| 361 | ataaaataaa ataaaagcaa tttcctttcc tctaagcggc ctccacccct ctcccctgcc |
| 421 | ctgtgaacgg gggaagctcc ggatcgcagc aattagggaa tttccccccg cgatgtcccg |
| 481 | gcacgccagt tcggcgcaca tctttcgctg cagtcctctt cctgctatct gtttactccc |
| 541 | taggcccctg gacctgggaa agagggaaag gcttcccgcc agctgcgcgg cgactccggg |
| 601 | gactccaggg cgcccctctg cggcgacgcc cgggtgcagc ggccgccggg ctggggccgg |
| 661 | cgggactccg cgggaccctc cagaagagcg gccggcggct gactcagcac tggggcggag |
| 721 | gggcgggaca cccttataag gctcggagcg cgagccttcg ctggagtttc gccgccgcag |
| 781 | tcttcgccac cagtgagtac gcgaccgcgt ccccggggat ggggctcaga gctccagcat |
| 841 | ggggccaacc cgcagcatca ggccgggctc ccggcggcct ccccacctcg agacccggga |
| 901 | cggggcctag gggacccagg acgtcccagt gccgttagcg gctttcaggg ggcccggagc |
| 961 | gcctcgggga gggatgggac cccgggggcg ggagggcagc tcactcaccg cgccttggca |
| 1021 | tcctccccgg gctccacaaa ttttctttgt tcgctgcagt gccgccctac accgtggtct |
| 1081 | atttcccagt tcgaggtagg agcatgtgtc tggcagggaa gggaggcagg ggctggggct |
| 1141 | gcagcaccca cagcccccac ccggagagat ccgaaccccc ttatccctcg tcgtgtgctt |
| 1201 | ttacccccgg cctccttcct gttccccgcc tctcccgcca tgcctgctcc ccgccccagt |
| 1261 | gttgtgtgaa atcttcggag gaacctgttt ccctgttccc tccctgcact cctgacccct |
| 1321 | ccccgggttg ctgcgaggcg gagtcggccc ggtccccaca tctcgtactt ctccctcccc |
| 1381 | gcaggccgct gcgcggccct gcgcatgctg ctggcagatc agggccagag ctggaaggag |
| 1441 | gaggtggtga ccgtggagac gtggcaggag agctcactca aagcctcctg cgtaagtgac |
| 1501 | catgcccggg caaggggagg gggtgctggg ccttaggggg ctgtgactag gatcggggga |
| 1561 | cgccccaagc tcagtgcccc tccctgagcc atgcctcccc caacagctat acgggcagct |
| 1621 | ccccaagttc caggacggag acctcaccct gtaccagtcc aataccatcc tgcgtcacct |
| 1681 | gggccgcacc cttggtgagt cttgaacctc caagtccagg gcaggcatgg gcaagcctct |
| 1741 | gcccccggag cccttttgtt taaatcagct gccccgcagc cctctggagt ggaggaaact |
| 1801 | gaaacccact gaggttacgt agtttgccca aagtcaagcc tgggtgcctg caatccttgc |
| 1861 | cctgtgccag gctgcctccc aggtgtcagg tgagctctga gcacctgctg tgtggcagtc |
| 1921 | tctcatcctt ccacgcacat cctcttcccc tcctcccagg ctggggctca cagacagccc |
| 1981 | cctggttggc ccatccccag tgactgtgtt gatcaggcgc ccagtcacgc ggcctgctcc |
| 2041 | cctccaccca accccagggc tctatgggaa ggaccagcag gaggcagccc tggtggacat |
| 2101 | ggtgaatgac ggcgtggagg acctccgctg caaatacatc tccctcatct acaccaacta |
| 2161 | tatgagcatc tgcaccaggg ttggacactg agggctgaac aaagaaaggg gcttcttgtg |
| 2221 | ccctcacccc ccttacccct caggtggctt gggctgaccc cttcttgggt cagggtgcag |
| 2281 | gggctgggtc agctctgggc caggggggcc tgggacaaga cacaacctgc acccttattg |
| 2341 | cctgggacat caaccaccca agtaacgggt catgggggcg agtgcaagga cagagacctc |
| 2401 | cagcaactgg tggtttctgc tctcctgggg tggccagagg tggaggagga tttgtgccag |
| 2461 | tttctggatg gagccgctgg cgcttttagc tgaggaaaat atgagacaca gagcactttg |
| 2521 | ggtaccaggg accagttcag cagaggcagc gtgtgtggcg tgtgtgtgcg tgtgtgtgcg |
| 2581 | tgtgtgtgtg tacgcttgca tttgtgtcgg gtgggtaagg agatagagat ggggcggcag |
| 2641 | taggcccagg tcccgaaggc cttgaaccca ctggtttgga gtctcctaag ggcaatgggg |
| 2701 | gccattgaga agtctgaaca gggctgtgtc tgaatgtgag gtctagaagg atcctccaga |
| 2761 | gaagccagct ctaaagcttt tgcaatcatc tggtgagaga acccaacaag gatagacagg |
| 2821 | cagaatggaa tagagatgag ttggcagctg aagtggacag gatttggtac tagcctggtt |
| 2881 | gtggggagca agcagaggag aatctgggac tctggtgtct ggcctggggc agacgggggt |
| 2941 | gtctcagggg ctgggaggga tgagagtagg atgatacatg gtgtgtgctg gcaggaggcg |
| 3001 | ggcaaggatg actatgtgaa ggcactgccc gggcaactga agccttttga gaccctgctg |
| 3061 | tcccagaacc agggaggcaa gaccttcatt gtgggagacc aggtgagcat ctggccccat |
| 3121 | gctgttcctt cctcgccacc ctctgcttcc agatggacac aggtgtgagc catttgttta |
| 3181 | gcaaagcaga gcagacctag gggatgggct taggccctct acccccaatt cctctccagc |
| 3241 | ctgctcccgc tggctgagtc cctagccccc ctgccctgca gatctccttc gctgactaca |
| 3301 | acctgctgga cttgctgctg atccatgagg tcctagcccc tggctgcctg gatgcgttcc |
| 3361 | ccctgctctc agcatatgtg gggcgcctca gtgcccggcc caagctcaag gccttcctgg |
| 3421 | cctcccctga gtacgtgaac ctccccatca atggcaacgg gaaacagtga gggttggggg |
| 3481 | gactctgagc gggaggcaga gtttgccttc ctttctccag gaccaataaa agggctaaga |
| 3541 | gagctactat gagcactgtg tttcctggga cggggcttag gggttctcag cctc |

A KRAS-associated malignant tumor or KRAS-associated cancer is defined herein as (a) a cancer cell or tumor cell containing a somatic KRAS mutation, or (b) a cancer cell or tumor cell with an abnormal expression level of KRAS including, but not limited to, amplification of the KRAS encoding DNA, or over-expression of the KRAS gene, or under-expression of the KRAS gene when compared to level found in normal, non-cancer cells.

Table 2 shows the amino acid sequence of the KRAS protein and identifies the mutations associated with cancer.

TABLE 2
Amino acid sequence of
KRAS protein and mutations associated with cancer
(SEQ ID NO: 2)
KRAS protein coding sequence, Isoform 2A (identifier: P01116-1)

```
                10 20 30 40 50
MTEYKLVVVG AGGVGKSALT IQLIQNHFVD EYDPTIEDSY RKQVVIDGET
                60 70 80 90 100
CLLDILDTAG QEEYSAMRDQ YMRTGEGFLC VFAINNTKSF EDIHHYREQI
              110 120 130 140 150
KRVKDSEDVP MVLVGNKCDL PSRTVDTKQA QDLARSYGIP FIETSAKTRQ
              160 170 180
RVEDAFYTLV REIRQYRLKK ISKEEKTPGC VKIKKCIIM
```

| | |
|---|---|
| Mutations at | G → A in a colorectal cancer sample |
| position 12: | G → C in lung carcinoma |
| | G → D in pancreatic carcinoma, GASC and lung |
| | carcinoma |
| | G → S in lung carcinoma and GASC |
| | G → V in lung carcinoma, pancreatic carcinoma, |
| | colon cancer and GASC |
| Mutations at | G → D in a breast carcinoma cell line and GASC |
| position 13: | G → R in pylocytic astrocytoma; amplification of |
| | the Ras pathway |
| Mutations at | Q → H in lung carcinoma |
| position 61: | Q → R in a colorectal cancer |

QIAGEN's THERASCREEN KRAS TEST is a genetic test designed to detect the presence of seven mutations in the KRAS gene in colorectal cancer cells.

Therapeutic Compositions

After a subject is diagnosed as having a neoplasia, e.g., a lung cancer or a pancreatic cancer, associated with a KRAS mutation or a KRAS amplification, a method of treatment involving suppression of GST-π is selected.

In one embodiment, the inhibitory nucleic acid molecules of the invention are administered systemically in dosages from about 1 to 100 mg/kg, e.g., 1, 5, 10, 20, 25, 50, 75, or 100 mg/kg.

1n further embodiments, the dosage can range from about 25 to 500 mg/m²/day.

Examples of an agent that suppresses GST-π as used herein include a drug that suppresses GST-π production and/or activity, and a drug that promotes GST-π degradation and/or inactivation. Examples of the drug that suppresses GST-π production include an RNAi molecule, a ribozyme, an antisense nucleic acid, a DNA/RNA chimera polynucleotide for DNA encoding GST-π, or a vector expressing same.

GST-pi and RNAi Molecules

One of ordinary skill in the art would understand that a reported sequence may change over time and to incorporate any changes needed in the nucleic acid molecules herein accordingly.

Embodiments of this invention can provide compositions and methods for gene silencing of GST-pi expression using small nucleic acid molecules. Examples of nucleic acid molecules include molecules active in RNA interference (RNAi molecules), short interfering RNA (siRNA), double-stranded RNA (dsRNA), micro-RNA (miRNA), and short hairpin RNA (shRNA) molecules. Such molecules are capable of mediating RNA interference against GST-pi gene expression.

The composition and methods disclosed herein can also be used in treating various kinds of malignant tumors in a subject.

The nucleic acid molecules and methods of this invention may be used to down regulate the expression of genes that encode GST-pi.

The compositions and methods of this invention can include one or more nucleic acid molecules, which, independently or in combination, can modulate or regulate the expression of GST-pi protein and/or genes encoding GST-pi proteins, proteins and/or genes encoding GST-pi associated with the maintenance and/or development of diseases, conditions or disorders associated with GST-pi, such as malignant tumor.

The compositions and methods of this invention are described with reference to exemplary sequences of GST-pi. A person of ordinary skill in the art would understand that various aspects and embodiments of the invention are directed to any related GST-pi genes, sequences, or variants, such as homolog genes and transcript variants, and polymorphisms, including single nucleotide polymorphism (SNP) associated with any GST-pi genes.

In some embodiments, the compositions and methods of this invention can provide a double-stranded short interfering nucleic acid (siRNA) molecule that downregulates the expression of a GST-pi gene, for example human GST-pi.

A RNAi molecule of this invention can be targeted to GST-pi and any homologous sequences, for example, using complementary sequences or by incorporating non-canonical base pairs, for example, mismatches and/or wobble base pairs, that can provide additional target sequences.

In instances where mismatches are identified, non-canonical base pairs, for example, mismatches and/or wobble bases can be used to generate nucleic acid molecules that target more than one gene sequence.

For example, non-canonical base pairs such as UU and CC base pairs can be used to generate nucleic acid molecules that are capable of targeting sequences for differing GST-pi targets that share sequence homology. Thus, a RNAi molecule can be targeted to a nucleotide sequence that is conserved between homologous genes, and a single RNAi molecule can be used to inhibit expression of more than one gene.

In some aspects, the compositions and methods of this invention include RNAi molecules that are active against GST-pi mRNA, where the RNAi molecule includes a sequence complementary to any mRNA encoding a GST-pi sequence.

In some embodiments, a RNAi molecule of this disclosure can have activity against GST-pi RNA, where the RNAi molecule includes a sequence complementary to an RNA having a variant GST-pi encoding sequence, for example, a mutant GST-pi gene known in the art to be associated with malignant tumor.

In further embodiments, a RNAi molecule of this invention can include a nucleotide sequence that can mediate silencing of GST-pi gene expression.

Examples of RNAi molecules of this invention targeted to GST-π mRNA are shown in Table 3.

TABLE 3
RNAi molecule sequences for GST-π

| | | SEQ | SENSE STRAND | SEQ | ANTISENSE STRAND |
|---|---|---|---|---|---|
| | Ref | ID | (5′-->3′) | ID | (5′-->3′) |
| ID | Pos | NO | SEQ ID NOS: 3 to 67 | NO | SEQ ID NOS: 68 to 132 |
| A1 | 652 | 3 | UCCCAGAACCAGGGAGGCAtt | 68 | UGCCUCCCUGGUUCUGGGAca |
| A10 | 635 | 4 | CUUUUGAGACCCUGCUGUCtt | 69 | GACAGCAGGGUCUCAAAAGgc |
| A11 | 649 | 5 | CUGUCCCAGAACCAGGGAGtt | 70 | CUCCCUGGUUCUGGGACAGca |
| A12 | 650 | 6 | UGUCCCAGAACCAGGGAGGtt | 71 | CCUCCCUGGUUCUGGGACAgc |
| A13 | 631 | 7 | AAGCCUUUUGAGACCCUGCtt | 72 | GCAGGGUCUCAAAAGGCUUca |
| A14 | 638 | 8 | UUGAGACCCUGCUGUCCCAtt | 73 | UGGGACAGCAGGGUCUCAAaa |
| A15 | 636 | 9 | UUUUGAGACCCUGCUGUCCtt | 74 | GGACAGCAGGGUCUCAAAAgg |
| A16 | 640 | 10 | GAGACCCUGCUGUCCCAGAtt | 75 | UCUGGGACAGCAGGGUCUCaa |
| A17 | 332 | 11 | GCUGGAAGGAGGAGGUGGUtt | 76 | ACCACCUCCUCCUUCCAGCtc |
| A18 | 333 | 12 | CUGGAAGGAGGAGGUGGUGtt | 77 | CACCACCUCCUCCUUCCAGct |
| A19 | 321 | 13 | UCAGGGCCAGAGCUGGAAGtt | 78 | CUUCCAGCUCUGGCCCUGAtc |
| A2 | 639 | 14 | UGAGACCCUGCUGUCCCAGtt | 79 | CUGGGACAGCAGGGUCUCAaa |
| A20 | 323 | 15 | AGGGCCAGAGCUGGAAGGAtt | 80 | UCCUUCCAGCUCUGGCCCUga |
| A21 | 331 | 16 | AGCUGGAAGGAGGAGGUGGtt | 81 | CCACCUCCUCCUUCCAGCUct |
| A22 | 641 | 17 | AGACCCUGCUGUCCCAGAAtt | 82 | UUCUGGGACAGCAGGGUCUca |
| A23 | 330 | 18 | GAGCUGGAAGGAGGAGGUGtt | 83 | CACCUCCUCCUUCCAGCUCtg |
| A25 | 647 | 19 | UGCUGUCCCAGAACCAGGGtt | 84 | CCCUGGUUCUGGGACAGCAgg |
| A26 | 653 | 20 | CCCAGAACCAGGGAGGCAAtt | 85 | UUGCCUCCCUGGUUCUGGGac |
| A3 | 654 | 21 | CCAGAACCAGGGAGGCAAGtt | 86 | CUUGCCUCCCUGGUUCUGGga |
| A4 | 637 | 22 | UUUGAGACCCUGCUGUCCCtt | 87 | GGGACAGCAGGGUCUCAAAag |
| A5 | 642 | 23 | GACCCUGCUGUCCCAGAACtt | 88 | GUUCUGGGACAGCAGGGUCtc |
| A6 | 319 | 24 | GAUCAGGGCCAGAGCUGGAtt | 89 | UCCAGCUCUGGCCCUGAUCtg |
| A7 | 632 | 25 | AGCCUUUUGAGACCCUGCUtt | 90 | AGCAGGGUCUCAAAAGGCUtc |
| A8 | 633 | 26 | GCCUUUUGAGACCCUGCUGtt | 91 | CAGCAGGGUCUCAAAAGGCtt |
| A9 | 634 | 27 | CCUUUUGAGACCCUGCUGUtt | 92 | ACAGCAGGGUCUCAAAAGGct |
| AG7 | 632 | 28 | CGCCUUUUGAGACCCUGCAtt | 93 | UGCAGGGUCUCAAAAGGCGtc |
| AK1 | 257 | 29 | CCUACACCGUGGUCUAUUUtt | 94 | AAAUAGACCACGGUGUAGGgc |
| AK10 | 681 | 30 | UGUGGGAGACCAGAUCUCCtt | 95 | GGAGAUCUGGUCUCCCACAat |
| AK11 | 901 | 31 | GCGGGAGGCAGAGUUUGCCtt | 96 | GGCAAACUCUGCCUCCCGCtc |
| AK12 | 922 | 32 | CCUUUCUCCAGGACCAAUAtt | 97 | UAUUGGUCCUGGAGAAAGGaa |
| AK13/ | 643 | 33 | ACCCUGCUGUCCCAGAACCtt | 98 | GGUUCUGGGACAGCAGGGUct |
| A24 | | | | | |
| AK2 | 267 | 34 | GGUCUAUUUCCCAGUUCGAtt | 99 | UCGAACUGGGAAAUAGACCac |
| AK3 | 512 | 35 | CCCUGGUGGACAUGGUGAAtt | 100 | UUCACCAUGUCCACCAGGGct |
| AK4 | 560 | 36 | ACAUCUCCCUCAUCUACACtt | 101 | GUGUAGAUGAGGGAGAUGUat |
| AK5 | 593 | 37 | GCAAGGAUGACUAUGUGAAtt | 102 | UUCACAUAGUCAUCCUUGCcc |
| AK6 | 698 | 38 | CCUUCGCUGACUACAACCUtt | 103 | AGGUUGUAGUCAGCGAAGGag |
| AK7 | 313 | 39 | CUGGCAGAUCAGGGCCAGAtt | 104 | UCUGGCCCUGAUCUGCCAGca |
| AK8 | 421 | 40 | GACGGAGACCUCACCCUGUtt | 105 | ACAGGGUGAGGUCUCCGUCct |
| AK9 | 590 | 41 | CGGGCAAGGAUGACUAUGUtt | 106 | ACAUAGUCAUCCUUGCCCGcc |
| AU10 | 635 | 42 | CUUUUGAGACCCUGCUGUAtt | 107 | UACAGCAGGGUCUCAAAAGgc |
| AU23 | 330 | 43 | GAGCUGGAAGGAGGAGGUAtt | 108 | UACCUCCUCCUUCCAGCUCtg |
| AU24 | 643 | 44 | ACCCUGCUGUCCCAGAACAtt | 109 | UGUUCUGGGACAGCAGGGUct |
| AU25 | 648 | 45 | UGCUGUCCCAGAACCAGGAtt | 110 | UCCUGGUUCUGGGACAGCAgg |
| AU7 | 632 | 46 | AGCCUUUUGAGACCCUGCAtt | 111 | UGCAGGGUCUCAAAAGGCUtc |
| AU9 | 634 | 47 | CCUUUUGAGACCCUGCUGAtt | 112 | UCAGCAGGGUCUCAAAAGGct |
| B1 | 629 | 48 | UGAAGCCUUUUGAGACCCUtt | 113 | AGGGUCUCAAAAGGCUUCAgt |
| B10 | 627 | 49 | ACUGAAGCCUUUUGAGACCtt | 114 | GGUCUCAAAAGGCUUCAGUtg |
| B11 | 595 | 50 | AAGGAUGACUAUGUGAAGGtt | 115 | CCUUCACAUAGUCAUCCUUgc |
| B12 | 596 | 51 | AGGAUGACUAUGUGAAGGCtt | 116 | GCCUUCACAUAGUCAUCCUtg |
| B13 | 597 | 52 | GGAUGACUAUGUGAAGGCAtt | 117 | UGCCUUCACAUAGUCAUCCtt |
| B14 | 564 | 53 | CUCCCUCAUCUACACCAACtt | 118 | GUUGGUGUAGAUGAGGGAGat |
| B2 | 630 | 54 | GAAGCCUUUUGAGACCCUGtt | 119 | CAGGGUCUCAAAAGGCUUCag |
| B3 | 563 | 55 | UCUCCCUCAUCUACACCAAtt | 120 | UUGGUGUAGAUGAGGGAGAtg |
| B4 | 567 | 56 | CCUCAUCUACACCAACUAUtt | 121 | AUAGUUGGUGUAGAUGAGGga |
| B5 | 566 | 57 | CCCUCAUCUACACCAACUAtt | 122 | UAGUUGGUGUAGAUGAGGGag |
| B6 | 625 | 58 | CAACUGAAGCCUUUUGAGAtt | 123 | UCUCAAAAGGCUUCAGUUGcc |
| B7 | 626 | 59 | AACUGAAGCCUUUUGAGACtt | 124 | GUCUCAAAAGGCUUCAGUUgc |
| B8 | 628 | 60 | CUGAAGCCUUUUGAGACCCtt | 125 | GGGUCUCAAAAGGCUUCAGtt |
| B9 | 565 | 61 | UCCCUCAUCUACACCAACUtt | 126 | AGUUGGUGUAGAUGAGGGAga |
| BG3 | 563 | 62 | GCUCCCUCAUCUACACCAAtt | 127 | UUGGUGUAGAUGAGGGAGCtg |
| BU2 | 630 | 63 | GAAGCCUUUUGAGACCCUAtt | 128 | UAGGGUCUCAAAAGGCUUCag |
| BU10 | 627 | 64 | ACUGAAGCCUUUUGAGACAtt | 129 | UGUCUCAAAAGGCUUCAGUtg |
| BU14 | 565 | 65 | CUCCCUCAUCUACACCAAAtt | 130 | UUUGGUGUAGAUGAGGGAGat |
| BU4 | 567 | 66 | CCUCAUCUACACCAACUAAtt | 131 | UUAGUUGGUGUAGAUGAGGga |
| C1- | 934 | 67 | ACCAAUAAAAUUUCUAAGAtt | 132 | UCUUAGAAAUUUUAUUGGUcc |
| 934 | | | | | |

Key for Table 3: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine respectively.

Examples of RNAi molecules of this invention targeted to GST-π mRNA are shown in Table 4.

TABLE 4
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 133 to 158 | NO | SEQ ID NOS: 159 to 184 |
| BU2′ | 133 | GAAGCCUUUUGAGACCCUANN | 159 | UAGGGUCUCAAAAGGCUUCNN |
| 14 | 134 | GAAGCCUUUUGAGACCCUAUU | 160 | UAGGGUCUCAAAAGGCUUCUU |
| 15 | 135 | GAAGCCUUUUGAGACCCUAUU | 161 | uagggucuCAAAAGGCUUCUU |
| 16 | 136 | GAAGCCUUUUGAGACCCUAUU | 162 | UagggucuCAAAAGGCUUCUU |
| 17 | 137 | GAAGCCUUUUGAGACCCUAUU | 163 | UAgggucuCAAAAGGCUUCUU |
| 18 | 138 | GAAGCCUUUUGAGACCCUAUU | 164 | UAGggucuCAAAAGGCUUCUU |
| 19 | 139 | GAAGCCUUUUGAGACCCUAUU | 165 | UAGGgucuCAAAAGGCUUCUU |
| 20 | 140 | GAAGCCUUUUGAGACCCUAUU | 166 | uAgGgUcUCAAAAGGCUUCUU |
| 21 | 141 | GAAGCCUUUUGAGACCCUAUU | 167 | UAgGgUcUCAAAAGGCUUCUU |
| 22 | 142 | GAAGCCUUUUGAGACCCUAUU | 168 | UaGgGuCuCAAAAGGCUUCUU |
| 23 | 143 | GAAGCCUUUUGAGACCCUAUU | 169 | UAGgGuCuCAAAAGGCUUCUU |
| 24 | 144 | GAAGCCUUUUGAGACCCUAtt | 170 | UagggucuCAAAAGGCUUCUU |
| 25 | 145 | GAAGCCUUUUGAGACCCUAUU | 171 | UAGGGUCUCAAAAGGCUUCUU |
| 26 | 146 | GAAGCCUUUUGAGACCCUAUU | 172 | fUAGGGUCUCAAAAGGCUUCUU |
| 27 | 147 | GAAGCCUUUUGAGACCCUAUU | 173 | uAGGGUCUCAAAAGGCUUCUU |
| 28 | 148 | GAAGCCUUUUGAGACCCUAUU | 174 | UsAGGGUCUCAAAAGGCUUCUU |
| 29 | 149 | GAAGCCUUUUGAGACCCUfAUU | 175 | fUAGGGUCUfCAAAAGGCfUUCUU |
| 30 | 150 | GAAGCCUUUUGAGfACCCUfAUU | 176 | fUAGGGUCUfCAfAfAAGGCfUUCUU |
| 31 | 151 | GAAGCCUUUUGAGACCCUAUU | 177 | UAGGGUCUCAAAAGGCUUCUU |
| 31′ | 152 | GAAGCCUUUUGAGACCCUAUU | 178 | fUAGGGUCUCAAAAGGCUUCUU |
| 32 | 153 | GAAGCCUUUUGAGACCCUAUU | 179 | UAGGGUCUCAAAAGGCUUCUU |
| 39 | 154 | GAAGCCUUUUGAGACCCUAUU | 180 | UAGgGuCuCAAAAGGCUUCUU |
| 45 | 155 | GAAGCCUUUUGAGACCCUAUU | 181 | UAGgGuCuCAAAAGGCUUCUU |
| 46 | 156 | GAAGCCUUUUGAGACCCUAUU | 182 | UAGgGuCuCAAAAGGCUUCUU |
| 47 | 157 | GAAGCCUUUUGAGACCCUAUU | 183 | UAGgGuCuCAAAAGGCUUCUU |
| 48 | 158 | GAAGCCUUUUGAGACCCUAUU | 184 | fUAGgGuCuCAAAAGGCUUCUU |

Key for Table 4: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U. The lower case letter f refers to 2′-deoxy-2′-fluoro substitution, e.g. fU is 2′-deoxy-2′-fluoro-U. N is A, C, G, U, U, a, c, g, u, t, or a modified, inverted, or chemically modified nucleotide.

Examples of RNAi molecules of this invention targeted to GST-π. mRNA are shown in Table 5.

TABLE 5
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 185 to 196 | NO | SEQ ID NOS: 197 to 208 |
| A9′ | 185 | CCUUUUGAGACCCUGCUGUNN | 197 | ACAGCAGGGUCUCAAAAGGNN |
| 1 | 186 | CCUUUUGAGACCCUGCUGUUU | 198 | ACAGCAGGGUCUCAAAAGGUU |
| 2 | 187 | CCUUUUGAGACCCUGCUGUUU | 199 | acagcaggGUCUCAAAAGGUU |
| 3 | 188 | CCUUUUGAGACCCUGCUGUUU | 200 | AcagcaggGUCUCAAAAGGUU |
| 4 | 189 | CCUUUUGAGACCCUGCUGUUU | 201 | ACagcaggGUCUCAAAAGGUU |
| 5 | 190 | CCUUUUGAGACCCUGCUGUUU | 202 | ACAgcaggGUCUCAAAAGGUU |
| 6 | 191 | CCUUUUGAGACCCUGCUGUUU | 203 | ACAGcaggGUCUCAAAAGGUU |
| 7 | 192 | CCUUUUGAGACCCUGCUGUUU | 204 | aCaGcAgGGUCUCAAAAGGUU |
| 8 | 193 | CCUUUUGAGACCCUGCUGUUU | 205 | ACaGcAgGGUCUCAAAAGGUU |
| 9 | 194 | CCUUUUGAGACCCUGCUGUUU | 206 | AcAgCaGgGUCUCAAAAGGUU |
| 10 | 195 | CCUUUUGAGACCCUGCUGUUU | 207 | ACAgCaGgGUCUCAAAAGGUU |
| 11 | 196 | CCUUUUGAGACCCUGCUGUUU | 208 | AcagcaggGUCUCAAAAGGUU |

Key for Table 5: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U. The lower case letter f refers to 2′-deoxy-2′-fluoro substitution, e.g., fU is 2′-deoxy-2′-fluoro-U. N is A, C, G, U, U, a, c, g, u, t, or a modified, inverted, or chemically modified nucleotide.

Examples of RNAi molecules of this invention targeted to GST-π mRNA are shown in Table 6.

TABLE 6
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 209 to 223 | NO | SEQ ID NOS: 224 to 238 |
| B13′ | 209 | GGAUGACUAUGUGAAGGCANN | 224 | UGCCUUCACAUAGUCAUCCNN |
| 4 | 210 | GGAUGACUAUGUGAAGGCAUU | 225 | UGCCUUCACAUAGUCAUCCUU |
| 5 | 211 | GGAUGACUAUGUGAAGGCAUU | 226 | ugccuucaCAUAGUCAUCCUU |
| 6 | 212 | GGAUGACUAUGUGAAGGCAUU | 227 | UgccuucaCAUAGUCAUCCUU |
| 7 | 213 | GGAUGACUAUGUGAAGGCAUU | 228 | UGccuucaCAUAGUCAUCCUU |
| 8 | 214 | GGAUGACUAUGUGAAGGCAUU | 229 | UGCcuucaCAUAGUCAUCCUU |
| 9 | 215 | GGAUGACUAUGUGAAGGCAUU | 230 | UGCCuucaCAUAGUCAUCCUU |
| 10 | 216 | GGAUGACUAUGUGAAGGCAUU | 231 | uGcCuUcACAUAGUCAUCCUU |
| 11 | 217 | GGAUGACUAUGUGAAGGCAUU | 232 | UGcCuUcACAUAGUCAUCCUU |
| 12 | 218 | GGAUGACUAUGUGAAGGCAUU | 233 | UgCcUuCaCAUAGUCAUCCUU |
| 13 | 219 | GGAUGACUAUGUGAAGGCAUU | 234 | UGCcUuCaCAUAGUCAUCCUU |
| 14 | 220 | GGAUGACUAUGUGAAGGCAUU | 235 | UgccuucaCAUAGUCAUCCUU |
| 15 | 221 | GGAUGACUAUfGUfGAAGGCAUU | 236 | UGCfCUUCACAUAGUCAUCCUU |
| 17 | 222 | GGAUGACUAUGUGAAGGCAUU | 237 | UGCCUUCACAUAGUCAUCCUU |
| 18 | 223 | GGAUGACUAUGUGAAGGCAUU | 238 | UGCCUUCACAUAGUCAUCCUU |

Key for Table 6: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U. The lower case letter f refers to 2′-deoxy-2′-fluoro substitution, e.g. fU is 2′-deoxy-2′-fluoro-U. N is A, C, G, U, U, a, c, g, u, t, or a modified, inverted, or chemically modified nucleotide.

Examples of RNAi molecules of this invention targeted to GST-π mRNA are shown in Table 7.

TABLE 7
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 239 to 250 | NO | SEQ ID NOS: 251 to 262 |
| B2′ | 239 | GAAGCCUUUUGAGACCCUGNN | 251 | CAGGGUCUCAAAAGGCUUCNN |
| 1 | 240 | GAAGCCUUUUGAGACCCUGUU | 252 | CAGGGUCUCAAAAGGCUUCUU |
| 2 | 241 | GAAGCCUUUUGAGACCCUGUU | 253 | cagggucuCAAAAGGCUUCUU |
| 3 | 242 | GAAGCCUUUUGAGACCCUGUU | 254 | CagggucuCAAAAGGCUUCUU |
| 4 | 243 | GAAGCCUUUUGAGACCCUGUU | 255 | CAgggucuCAAAAGGCUUCUU |
| 5 | 244 | GAAGCCUUUUGAGACCCUGUU | 256 | CAGggucuCAAAAGGCUUCUU |
| 6 | 245 | GAAGCCUUUUGAGACCCUGUU | 257 | CAGGgucuCAAAAGGCUUCUU |
| 7 | 246 | GAAGCCUUUUGAGACCCUGUU | 258 | cAgGgUcUCAAAAGGCUUCUU |
| 8 | 247 | GAAGCCUUUUGAGACCCUGUU | 259 | CAgGgUcUCAAAAGGCUUCUU |
| 9 | 248 | GAAGCCUUUUGAGACCCUGUU | 260 | CaGgGuCuCAAAAGGCUUCUU |
| 10 | 249 | GAAGCCUUUUGAGACCCUGUU | 261 | CAGgGuCuCAAAAGGCUUCUU |
| 11 | 250 | GAAGCCUUUUGAGACCCUGUU | 262 | CagggucuCAAAAGGCUUCUU |

Key for Table 7: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U. The lower case letter Prefers to 2′-deoxy-2′-fluoro substitution, e.g. fU is 2′-deoxy-2′-fluoro-U. N is A, C, G, U, U, a, c, g, u, t, or a modified, inverted, or chemically modified nucleotide.

Examples of RNAi molecules of this invention targeted to GST-π mRNA are shown in Table 8.

TABLE 8
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 263 to 274 | NO | SEQ ID NOS: 275 to 286 |
| B4′ | 263 | CCUCAUCUACACCAACUAUNN | 275 | AUAGUUGGUGUAGAUGAGGNN |
| 1 | 264 | CCUCAUCUACACCAACUAUUU | 276 | AUAGUUGGUGUAGAUGAGGUU |
| 2 | 265 | CCUCAUCUACACCAACUAUUU | 277 | auaguuggUGUAGAUGAGGUU |
| 3 | 266 | CCUCAUCUACACCAACUAUUU | 278 | AuaguuggUGUAGAUGAGGUU |
| 4 | 267 | CCUCAUCUACACCAACUAUUU | 279 | AUaguuggUGUAGAUGAGGUU |
| 5 | 268 | CCUCAUCUACACCAACUAUUU | 280 | AUAguuggUGUAGAUGAGGUU |
| 6 | 269 | CCUCAUCUACACCAACUAUUU | 281 | AUAGuuggUGUAGAUGAGGUU |
| 7 | 270 | CCUCAUCUACACCAACUAUUU | 282 | aUaGuUgGUGUAGAUGAGGUU |
| 8 | 271 | CCUCAUCUACACCAACUAUUU | 283 | AUaGuUgGUGUAGAUGAGGUU |
| 9 | 272 | CCUCAUCUACACCAACUAUUU | 284 | AuAgUuGgUGUAGAUGAGGUU |
| 10 | 273 | CCUCAUCUACACCAACUAUUU | 285 | AUAgUuGgUGUAGAUGAGGUU |
| 11 | 274 | CCUCAUCUACACCAACUAUUU | 286 | AuaguuggUGUAGAUGAGGUU |

Key for Table 8: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U. The lower case letter f refers to 2′-deoxy-2′-fluoro substitution, e.g. fU is 2′-deoxy-2′-fluoro-U. N is A, C G, U, U, a, c, g, u, t, or a modified, inverted, or chemically modified nucleotide.

As used herein, the RNAi molecule denotes any molecule that causes RNA interference, including, but not limited to, a duplex RNA such as siRNA (small interfering RNA), miRNA (micro RNA), shRNA (short hairpin RNA), ddRNA (DNA-directed RNA), piRNA (Piwi-interacting RNA), or rasiRNA (repeat associated siRNA) and modified forms thereof. These RNAi molecules may be commercially available or may be designed and prepared based on known sequence information, etc. The antisense nucleic acid includes RNA, DNA, PNA, or a complex thereof. As used herein, the DNA RNA chimera polynucleotide includes, but is not limited to, a double-strand polynucleotide composed of DNA and RNA that inhibits the expression of a target gene.

In one embodiment, the agents of this invention contain siRNA as a therapeutic agent. An siRNA molecule can have a length from about 10-50 or more nucleotides. An siRNA molecule can have a length from about 15-45 nucleotides. An siRNA molecule can have a length from about 19-40 nucleotides. An siRNA molecule can have a length of from 19-23 nucleotides. An siRNA molecule of this invention can mediate RNAi against a target mRNA. Commercially available design tools and kits, such as those available from Ambion, Inc. (Austin, Tex.), and the Whitehead Institute of Biomedical Research at MIT (Cambridge, Mass.) allow for the design and production of siRNA.

Methods for Modulating GST-pi and Treating Malignant Tumor

Embodiments of this invention can provide RNAi molecules that can be used to down regulate or inhibit the expression of GST-pi and/or GST-pi proteins.

In some embodiments, a RNAi molecule of this invention can be used to down regulate or inhibit the expression of GST-pi and/or GST-pi proteins arising from GST-pi haplotype polymorphisms that may be associated with a disease or condition such as malignant tumor.

Monitoring of GST-pi protein or mRNA levels can be used to characterize gene silencing, and to determine the efficacy of compounds and compositions of this invention.

The RNAi molecules of this disclosure can be used individually, or in combination with other siRNAs for modulating the expression of one or more genes.

The RNAi molecules of this disclosure can be used individually, or in combination, or in conjunction with other known drugs for preventing or treating diseases, or ameliorating symptoms of conditions or disorders associated with GST-pi, including malignant tumor.

The RNAi molecules of this invention can be used to modulate or inhibit the expression of GST-pi in a sequence-specific manner.

The RNAi molecules of this disclosure can include a guide strand for which a series of contiguous nucleotides are at least partially complementary to a GST-pi mRNA.

In certain aspects, malignant tumor may be treated by RNA interference using a RNAi molecule of this invention.

Treatment of malignant tumor may be characterized in suitable cell-based models, as well as ex vivo or in vivo animal models.

Treatment of malignant tumor may be characterized by determining the level of GST-pi mRNA or the level of GST-pi protein in cells of affected tissue.

Treatment of malignant tumor may be characterized by non-invasive medical scanning of an affected organ or tissue.

Embodiments of this invention may include methods for preventing, treating, or ameliorating the symptoms of a GST-pi associated disease or condition in a subject in need thereof.

In some embodiments, methods for preventing, treating, or ameliorating the symptoms of malignant tumor in a subject can include administering to the subject a RNAi molecule of this invention to modulate the expression of a GST-pi gene in the subject or organism.

In some embodiments, this invention contemplates methods for down regulating the expression of a GST-pi gene in a cell or organism, by contacting the cell or organism with a RNAi molecule of this invention.

GST-π inhibitory nucleic acid molecules can be nucleotide oligomers that may be employed as single-stranded or double-stranded nucleic acid molecule to decrease GST-π expression. In one approach, the GST-π inhibitory nucleic acid molecule is a double-stranded RNA used for RNA interference (RNAi)-mediated knockdown of GST-π gene expression. In one embodiment, a double-stranded RNA (dsRNA) molecule is made that includes from eight to twenty-five (e.g., 8, 10, 12, 15, 16, 17, 18, 19, 20, 21, 22, 23, 24, 25) consecutive nucleotides of a nucleotide oligomer of the invention. The dsRNA can be two complementary strands of RNA that have duplexed, or a single RNA strand that has self-duplexed (small hairpin (sh)RNA).

In some embodiments, dsRNAs are about 21 or 22 base pairs, but may be shorter or longer, up to about 29 nucleotides. Double stranded. RNA can be made using standard techniques, e.g., chemical synthesis or in vitro transcription. Kits are available, for example, from Ambion (Austin, Tex.) and Epicentre (Madison, Wis.).

Methods for expressing dsDNA in mammalian cells are described in Brummelkamp et al. Science 296:550-553, 2002; Paddison et al. Genes & Devel. 16:948-958, 2002; Paul et al. Nature Biotechnol, 20:505-508, 2002; Sui et al., Proc. Natl. Acad. Sci. USA 99:5515-5520, 2002; Yu et al. Proc. Natl. Acad. Sci. USA 99:6047-6052, 2002; Miyagishi et al., Nature Biotechnol, 20:497-500, 2002; and Lee et at, Nature Biotechnol. 20:500-505 2002, each of which is hereby incorporated by reference.

An inhibitory nucleic acid molecule that “corresponds” to a GST-π gene comprises at least a fragment of the double-stranded gene, such that each strand of the double-stranded inhibitory nucleic acid molecule is capable of binding to the complementary strand of the target GST-π gene. The inhibitory nucleic acid molecule need not have perfect correspondence to the reference GST-π sequence.

In one embodiment, a siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleotide sequence of the inhibitory nucleic acid molecule exhibits 1, 2, 3, 4, 5 or more mismatches.

The inhibitory nucleic acid molecules provided by the invention are not limited to siRNAs, but include any nucleic acid molecule sufficient to decrease the expression of a GST-π nucleic acid molecule or polypeptide. Each of the DNA sequences provided herein may be used, for example, in the discovery and development of therapeutic anti sense nucleic acid molecule to decrease the expression of GST-π. The invention further provides catalytic RNA molecules or ribozymes. Such catalytic RNA molecules can be used to inhibit expression of an GST-π nucleic acid molecule in vivo. The inclusion of ribozyme sequences within an antisense RNA confers RNA-cleaving activity upon the molecule, thereby increasing the activity of the constructs. The design and use of target RNA-specific ribozymes is described in Haseloff et al., Nature 334:585-591. 1988, and US 2003/0003469 A1, each of which is incorporated by reference.

In various embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., Biochemistry, 28:4929, 1989, and Hampel et al., Nucleic Acids Research, 18: 299, 1990. Those skilled in the art will recognize that what is needed in an enzymatic nucleic acid molecule is a specific substrate binding site that is complementary to one or more of the target gene RNA regions, and that it have nucleotide sequences within or surrounding that substrate binding site which impart an RNA cleaving activity to the molecule.

Table 9 shows the mRNA coding sequence of GST-π.

TABLE 9
Glutathione S-transferase-π1 mRNA coding sequence,
NCBI Reference Sequence: NM_000852.3,
GeneID: 2950, Hugo gene Nomenclature Committee:
HGNC: 4638, Human Protein Reference Database:
HPRD: 00614 (SEQ ID NO: 287)

| | |
|---|---|
| 1 | tgggaaagag ggaaaggctt ccccggccag ctgcgcggcg |
| | actccgagga ctccagggcg |
| 61 | cccctctgcg gccgacgccc ggggtgcagc ggccgccggg |
| | gctggggccg gcgggagtcc |
| 121 | gcgggaccct ccagaagagc ggccggcgcc gtgactcagc |
| | actgaggcgg agcgaggcgg |
| 181 | gaccaccctt ataaggctcg gaggccgcga ggccttcgct |
| | ggaatttcgc cgccgcagtc |
| 241 | ttcgccacca tgccgcccta caccgtagtc tatttcccag |
| | ttcgaggcca ctgcgcggcc |
| 301 | ctgcgcatgc tgctggcaga tcagggccag agctggaagg |
| | aggaggtgat gaccgtggag |
| 361 | acgtggcagg agggctcact caaagcctcc tgcctatacg |
| | ggcagctccc caagttccag |
| 421 | gacggagacc tcaccctgta ccagtccaat accatcctgc |
| | gtcacctggg ccgcaccctt |
| 481 | gggctctatg ggaaggacca gcaggaggca gccctggtgg |
| | acatggtgaa tgacggcgtg |
| 541 | gaggacctcc gctgcaaata catctccctc atctacacca |
| | actatgaggc gggcaaggat |
| 601 | gactatgtga agacactgcc cgggcaactg aagccttttg |
| | agaccctgct gtcccaaaac |
| 661 | cagggaggca agaccttcat tgtgagagac cagatctcct |
| | tcgctgacta caacctgctg |
| 721 | gacttgctgc tgatccatga ggtcctagcc cctggctgcc |
| | tggatgcgtt ccccctgctc |
| 781 | tcagcatatg tggggcgcct cagtgcccgg cccaagctca |
| | aggccttcct ggcctcccct |
| 841 | gagtacgtga acctccccat caatggcaac gggaaacagt |
| | gaggattggg gggactctga |
| 901 | gcgggaggca gaatttgcct tcatttctcc aggaccaata |
| | aaatttctaa gagagctaaa |
| 961 | aaaaaaaaaa aaaaaaaaaa aaaaaa |

The drug that suppresses GST-π production or activity can be an RNAi molecule, a ribozyme, an antisense nucleic acid, a DNA/RNA chimera polynucleotide for DNA encoding GST-π, or a vector expressing same, in terms of high specificity and a low possibility of side effects.

Suppression of GST-π may be determined by the expression or activity of GST-π in cells being suppressed compared with a case in which a GST-π suppressing agent is not utilized. Expression of GST-π may be evaluated by any known technique; examples thereof include an immunoprecipitation method utilizing an anti-GST-π antibody, EIA, ELISA, IRA, IRMA, a western blot method, an immunohistochemical method, an immunocytochemical method, a flow cytometry method, various hybridization methods utilizing a nucleic acid that specifically hybridizes with a nucleic acid encoding GST-π or a unique fragment thereof, or a transcription product (e.g., mRNA) or splicing product of said nucleic acid, a northern blot method, a Southern blot method, and various PCR methods.

The activity of GST-π may be evaluated by analyzing a known activity of GST-π including binding to a protein such as, for example, Raf-1 (in particular phosphorylated Raf-1) or EGFR (in particular phosphorylated EGFR) by means of any known method such as for example an immunoprecipitation method, a western blot method, amass analysis method, a pull-down method, or a surface plasmon resonance (SPR) method.

Whether or not GST-π is being expressed in certain cells may be determined by detecting expression of GST-π in cells. Expression of GST-π may be detected by any technique known in the art.

Examples of the mutated KRAS include, but are not limited to, those having a mutation that causes constant activation of KRAS, such as a mutation that inhibits endogenous GTPase or a mutation that increases the guanine nucleotide exchange rate. Specific examples of such mutation include, but are not limited to, for example, mutation in amino acids 12, 13 and/or 61 in human KRAS (inhibiting endogenous GTPase) and mutation in amino acids 116 and/or 119 in human KRAS (increasing guanine nucleotide exchange rate) (Bos, Cancer Res. 1989; 49 (17): 4682-9, Levi et al., Cancer Res. 1991; 51 (13): 3497-502).

In some embodiments of the present invention, the mutated KRAS can be a KRAS having a mutation in at least one of amino acids 12; 13, 61, 116, and 119 of human KRAS. In one embodiment of the present invention, the mutated KRAS has a mutation at amino acid 12 of human KRAS. In some embodiments, the mutated KRAS may be one that induces overexpression of GST-π. Cells having mutated KRAS may exhibit overexpression of GST-π.

Detection of mutated KRAS may be carried out using any known technique, e.g., selective hybridization by means of a nucleic acid probe specific to a known mutation sequence, an enzyme mismatch cleavage method, sequencing (Bos, Cancer Res. 1989; 49 (17): 4682-9), and a PCR-RFLP method (Miyanishi et al., Gastroenterology. 2001; 121 (4): 865-74)).

Detection of GST-π expression may be carried out using any known technique. Whether or not GST-π is being overexpressed may be evaluated by for example comparing the degree of expression of GST-π in cells having mutated KRAS with the degree of expression of GST-π in the same type of cells having normal KRAS. In this situation, GST-π is being overexpressed if the degree of expression of GST-π in cells having mutated KRAS exceeds the degree of expression of GST-π in the same type of cells having normal KRAS.

In one aspect, the invention features a vector encoding an inhibitory nucleic acid molecule of any of the above aspects. In a particular embodiment, the vector is a retroviral, adenoviral, adeno-associated viral, or lentiviral vector. In another embodiment, the vector contains a promoter suitable for expression in a mammalian cell.

The amount of active RNA interference inducing ingredient formulated in the composition of the present invention may be an amount that does not cause an adverse effect exceeding the benefit of administration. Such an amount may be determined by an in vitro test using cultured cells, or a test in a model animal such as a mouse, a rat, a dog, or a pig, etc., and such test methods are well known to a person skilled in the art.

The amount of active ingredient formulated can vary according to the manner in which the agent or composition is administered. For example, when a plurality of units of the composition is used for one administration, the amount of active ingredient to be formulated in one unit of the composition may be determined by dividing the amount of active ingredient necessary for one administration by said plurality of units.

This invention also relates to a process for producing an agent or composition for suppressing GST-π, and the use of a drug that suppresses GST-π in the production of an agent or composition for reducing or shrinking malignant tumors.

RNA Interference

RNA interference (RNAi) refers to sequence-specific post-transcriptional gene silencing in animals mediated by short interfering RNAs (siRNAs). See, e.g., Zamore et al., Cell, 2000, Vol. 101, pp. 25-33; Fire et al., Nature, 1998, Vol. 391, pp. 806811; Sharp, Genes & Development, 1999, Vol, 13, pp. 139-141.

An RNAi response in cells can be triggered by a double stranded RNA (dsRNA), although the mechanism is not yet fully understood. Certain dsRNAs in cells can undergo the action of Dicer enzyme, a ribonuclease III enzyme, See, e.g., Zamore et al., Cell, 2000, Vol. 101, pp. 25-33; Hammond et al., Nature, 2000, Vol. 404, pp. 293-296. Dicer can process the dsRNA into shorter pieces of dsRNA, which are siRNAs.

In general, siRNAs can be from about 21 to about 23 nucleotides in length and include a base pair duplex region about 19 nucleotides in length.

RNAi involves an endonuclease complex known as the RNA induced silencing complex (RISC). An siRNA has an antisense or guide strand which enters the RISC complex and mediates cleavage of a single stranded RNA target having a sequence complementary to the antisense strand of the siRNA duplex. The other strand of the siRNA is the passenger strand. Cleavage of the target RNA takes place in the middle of the region complementary to the anti sense strand of the siRNA duplex See, e.g., Elbashir et al., Genes & Development, 2001, Vol. 15, pp. 188-200.

As used herein, the term “sense strand” refers to a nucleotide sequence of a siRNA molecule that is partially or fully complementary to at least a portion of a corresponding antisense strand of the siRNA molecule. The sense strand of a siRNA molecule can include a nucleic acid sequence having homology with a target nucleic acid sequence.

As used herein, the term “antisense strand” refers to a nucleotide sequence of a siRNA molecule that is partially or fully complementary to at least a portion of a target nucleic acid sequence. The antisense strand of a siRNA molecule can include a nucleic acid sequence that is complementary to at least a portion of a corresponding sense strand of the siRNA molecule.

RNAi molecules can down regulate or knock down gene expression by mediating RNA interference in a sequence-specific manner. See, e.g., Zamore et al., Cell, 2000, Vol. 101, pp. 25-33; Elbashir et al., Nature, 2001, Vol. 411, pp. 494-498; Kreutzer et al., WO2000/044895; Zemicka-Goetz et al., WO2001/36646; Fire et al., WO1999/032619; Plaetinck et al, WO2000/01846; Mello et al., WO2001/029058.

As used herein, the terms “inhibit,” “down-regulate,” or “reduce” with respect to gene expression means that the expression of the gene, or the level of mRNA molecules encoding one or more proteins, or the activity of one or more of the encoded proteins is reduced below that observed in the absence of a RNAi molecule or siRNA of this invention. For example, the level of expression, level of mRNA, or level of encoded protein activity may be reduced by at least 1%, or at least 10%, or at least 20%, or at least 50%, or at least 90%, or more from that observed in the absence of a RNAi molecule or siRNA of this invention.

RNAi molecules can also be used to knock down viral gene expression, and therefore affect viral replication.

RNAi molecules can be made from separate polynucleotide strands: a sense strand or passenger strand, and an antisense strand or guide strand. The guide and passenger strands are at least partially complementary. The guide strand and passenger strand can form a duplex region having from about 15 to about 49 base pairs.

In some embodiments, the duplex region of a siRNA can have 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 base pairs.

In certain embodiments, a RNAi molecule can be active in a RISC complex, with a length of duplex region active for RISC.

In additional embodiments, a RNAi molecule can be active as a Dicer substrate, to be converted to a RNAi molecule that can be active in a RISC complex.

In some aspects, a RNAi molecule can have complementary guide and passenger sequence portions at opposing ends of a long molecule, so that the molecule can form a duplex region with the complementary sequence portions, and the strands are linked at one end of the duplex region by either nucleotide or non-nucleotide linkers. For example, a hairpin arrangement, or a stem and loop arrangement. The linker interactions with the strands can be covalent bonds or non-covalent interactions.

A RNAi molecule of this disclosure may include a nucleotide, non-nucleotide, or mixed nucleotide/non-nucleotide linker that joins the sense region of the nucleic acid to the antisense region of the nucleic acid. A nucleotide linker can be a linker of ≥2 nucleotides in length, for example about 3, 4, 5, 6, 7, 8, 9, or 10 nucleotides in length. The nucleotide linker can be a nucleic acid aptamer. By “aptamer” or “nucleic acid aptamer” as used herein refers to a nucleic acid molecule that binds specifically to a target molecule wherein the nucleic acid molecule has sequence that includes a sequence recognized by the target molecule in its natural setting. Alternately, an aptamer can be a nucleic acid molecule that binds to a target molecule, where the target molecule does not naturally bind to a nucleic acid. For example, the aptamer can be used to bind to a ligand-binding domain of a protein, thereby preventing interaction of the naturally occurring ligand with the protein. See, e.g., Gold et al., Annu Rev Biochem, 1995, Vol. 64, pp. 763-797; Brody et at, J. Biotechnol., 2000, Vol. 74, pp, 5-13; Hermann et al., Science, 2000 Vol. 287, pp. 820-825.

Examples of a non-nucleotide linker include an abasic nucleotide, polyether, polyamine, polyimide, peptide, carbohydrate, lipid, polyhydrocarbon, or other polymeric compounds, for example polyethylene glycols such as those having from 2 to 100 ethylene glycol units. Some examples are described in Seela et al., Nucleic Acids Research, 1987, Vol. 15, pp. 3113-3129; Cload et al., J. Am. Chem. Soc., 1991, Vol. 113, pp. 6324-6326; Jaeschke et al., Tetrahedron Lett., 1993, Vol. 34, pp. 301; Arnold et al., WO1989/002439; Usman et al., WO1995/006731; Dudycz et al., WO1995/011910, and Ferentz et al., J. Am. Chem. Soc., 1991, Vol. 113, pp. 4000-4002.

A RNAi molecule can have one or more overhangs from the duplex region. The overhangs, which are non-base-paired, single strand regions, can be from one to eight nucleotides in length, or longer. An overhang can be a 3′-end overhang, wherein the 3′-end of a strand has a single strand region of from one to eight nucleotides. An overhang can be a 5′-end overhang, wherein the 5′-end of a strand has a single strand region of from one to eight nucleotides.

The overhangs of a RNAi molecule can have the same length, or can be different lengths.

A RNAi molecule can have one or more blunt ends, in which the duplex region ends with no overhang, and the strands are base paired to the end of the duplex region.

A RNAi molecule of this disclosure can have one or more blunt ends, or can have one or more overhangs, or can have a combination of a blunt end and an overhang end.

A 5′-end of a strand of a RNAi molecule may be in a blunt end, or can be in an overhang. A 3′-end of a strand of a RNAi molecule may be in a blunt end, or can be in an overhang.

A 5′-end of a strand of a RNAi molecule may be in a blunt end, while the 3?-end is in an overhang. A3′-end of a strand of a RNAi molecule may be in a blunt end, while the 5′-end is in an overhang.

In some embodiments, both ends of a RNAi molecule are blunt ends.

In additional embodiments, both ends of a RNAi molecule have an overhang.

The overhangs at the 5′- and 3′-ends may be of different lengths.

In certain embodiments, a RNAi molecule may have a blunt end where the 5′-end of the anti sense strand and the 3′-end of the sense strand do not have any overhanging nucleotides.

In further embodiments, a RNAi molecule may have a blunt end where the 3′-end of the antisense strand and the 5′-end of the sense strand do not have any overhanging nucleotides.

A RNAi molecule may have mismatches in base pairing in the duplex region.

Any nucleotide in an overhang of a RNAi molecule can be a deoxyribonucleotide, or a ribonucleotide.

One or more deoxyribonucleotides may be at the 5′-end, where the 3′-end of the other strand of the RNAi molecule may not have an overhang, or may not have a deoxyribonucleotide overhang.

One or more deoxyribonucleotides may be at the 3′-end, where the 5′-end of the other strand of the RNAi molecule may not have an overhang, or may not have a deoxyribonucleotide overhang.

In some embodiments, one or more, or all of the overhang nucleotides of a RNAi molecule may be 2′-deoxyribonucleotides.

Dicer Substrate RNAi Molecules

in some aspects, a RNAi molecule can be of a length suitable as a Dicer substrate, which can be processed to produce a RISC active RNAi molecule. See, e.g., Rossi et al., US2005/0244858.

A Dicer substrate dsRNA can be of a length sufficient such that it is processed by Dicer to produce an active RNAi molecule, and may further include one or more of the following properties: (i) the Dicer substrate dsRNA can be asymmetric, for example, having a 3′ overhang on the anti sense strand, and (ii) the Dicer substrate dsRNA can have a modified 3′ end on the sense strand to direct orientation of Dicer binding and processing of the dsRNA to an active RNAi molecule.

Methods of Use of RNAi Molecules

The nucleic acid molecules and RNAi molecules of this invention may be delivered to a cell or tissue by direct application of the molecules, or with the molecules combined with a carrier or a diluent.

The nucleic acid molecules and RNAi molecules of this invention can be delivered or administered to a cell, tissue, organ, or subject by direct application of the molecules with a carrier or diluent, or any other delivery vehicle that acts to assist, promote or facilitate entry into a cell, for example, viral sequences, viral material, or lipid or liposome formulations.

The nucleic acid molecules and RNAi molecules of this invention can be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection.

Delivery systems may include, for example, aqueous and nonaqueous gels, creams, emulsions, microemulsions, liposomes, ointments, aqueous and nonaqueous solutions, lotions, aerosols, hydrocarbon bases and powders, and can contain excipients such as solubilizers and permeation enhancers.

A GST-π inhibitory nucleic acid molecule of this invention may be administered within a pharmaceutically-acceptable diluents, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Compositions and methods of this disclosure can include an expression vector that includes a nucleic acid sequence encoding at least one RNAi molecule of this invention in a manner that allows expression of the nucleic acid molecule.

The nucleic acid molecules and RNAi molecules of this invention can be expressed from transcription units inserted into DNA or RNA vectors. Recombinant vectors can be DNA plasmids or viral vectors. Viral vectors can be used that provide for transient expression of nucleic acid molecules.

For example, the vector may contain sequences encoding both strands of a RNAi molecule of a duplex, or a single nucleic acid molecule that is self-complementary and thus forms a RNAi molecule. An expression vector may include a nucleic acid sequence encoding two or more nucleic acid molecules.

A nucleic acid molecule may be expressed within cells from eukaryotic promoters. Those skilled in the art realize that any nucleic acid can be expressed in eukaryotic cells from the appropriate DNA/RNA vector.

In some aspects, a viral construct can be used to introduce an expression construct into a cell, for transcription of a dsRNA construct encoded by the expression construct.

Lipid formulations can be administered to animals by intravenous, intramuscular, or intraperitoneal injection, or orally or by inhalation or other methods as are known in the art.

Pharmaceutically acceptable formulations for administering oligonucleotides are known and can be used.

In one embodiment of the above method, the inhibitory nucleic acid molecule is administered at a dosage of about 5 to 500 mg/m²/day, e.g., 5, 25, 50, 100, 125, 150, 175, 200, 225, 250, 275, or 300 mg/m²/day.

Methods known in the art for making formulations are found, for example, in “Remington: The Science and Practice of Pharmacy” Ed. A. R. Gennaro, Lippincourt Williams & Wilkins, Philadelphia, Pa., 2000.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for GST-π inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition. The preferred dosage of a nucleotide oligomer of the invention can depend on such variables as the type and extent of the disorder, the overall health status of the particular patient, the formulation of the compound excipients, and its route of administration.

All of the above methods for reducing malignant tumors may be either an in vitro method or an in vivo method. Dosage may be determined by an in vitro test using cultured cells, etc., as is known in the art. An effective amount may be an amount that reduces tumor size in KRAS associated tumors by at least 10%, at least 20%, or at least 30%, or at least 40%, or at least 50%, or at least 60%, or at least 70%, or at least 80%, or at least 90%, up to 100% of the tumor size.

A pharmaceutical composition of this invention can be effective in treating a KRAS associated disease. Examples of the diseases include a disease due to abnormal cell proliferation, a disease due to KRAS mutation, and a disease due to GST-π overexpression.

Examples of the disease due to abnormal cell proliferation include malignant tumors, hyperplasia, keloid, Cushing's syndrome, primary aldosteronism erythroplakia, polycythemia vera, leukoplakia, hyperplastic scar, lichen planus, and lentiginosis.

Examples of the disease due to KRAS mutation include malignant tumor (also called a cancer or a malignant neoplasm).

Examples of the disease due to GST-π overexpression include malignant tumor.

Examples of cancer include sarcomas such as fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, and osteosarcoma, carcinomas such as brain tumor, head and neck carcinoma, breast carcinoma, lung carcinoma, esophageal carcinoma, gastric carcinoma, duodenal carcinoma, colon carcinoma, rectal carcinoma, liver carcinoma, pancreatic carcinoma, gall bladder carcinoma, bile duct carcinoma, renal carcinoma, ureteral carcinoma, bladder carcinoma, prostate carcinoma, testicular carcinoma, uterine carcinoma, ovarian carcinoma, skin carcinoma, leukemia, and malignant lymphoma.

Cancer includes epithelial malignancy and non-epithelial malignancy. A cancer can be present at any site of the body, for example, the brain, head and neck, chest, limbs, lung, heart, thymus, esophagus, stomach, small intestine (duodenum, jejunum, ileum), large intestine (colon, cecum, appendix, rectum), liver, pancreas, gallbladder, kidney, urinary duct, bladder, prostate, testes, uterus, ovary, skin, striated muscle, smooth muscle, synovial membrane, cartilage, hone, thyroid, adrenal gland, peritoneum, mesentery, bone marrow, blood, vascular system, lymphatic system such as lymph node, lymphatic fluid, etc.

In one embodiment of the present invention, the cancer includes cancer cells having the mutated KISS defined above. In another embodiment, the cancer includes cancer cells that exhibit hormone- or growth factor-independent proliferation. In further embodiments, a cancer includes cancer cells exhibiting GST-π overexpression.

### EXAMPLES

Example 1: siRNAs of this invention targeted to GST-π were found to be active for gene silencing in vitro. The dose-dependent activities of GST-π siRNAs for gene knockdown were found to exhibit an IC50 below about 250 picomolar (pM), and as low as 1 pM.

In vitro transfection was performed in an A549 cell line to determine siRNA knockdown efficacy. Dose dependent knockdown for GST-πmRNA was observed with siRNAs of Table 3, as shown in Table 10.

TABLE 10
Dose dependent knockdown for GST-π mRNA in an A549 cell line

| siRNA structure | IC50 (pM) |
|---|---|
| A9 (SEQ ID NOs: 27 and 92) | 24 |
| B2 (SEQ ID NOs: 54 and 119) | 121 |
| B3 (SEQ ID NOs: 55 and 120) | 235 |
| B4 (SEQ ID NOs: 56 and 121) | 229 |
| B13 (SEQ ID NOs: 52 and 117) | 17 |
| BU2 (SEQ ID NOs: 63 and 128) | 31 |

As shown in Table 10, the activities of GST-π siRNAs of Table 3 were in the range 17-235 pM, which is suitable for many uses, including as a drug agent to be used in vivo.

Example 2: The structure of GST-π siRNAs of this invention having deoxynucleotides located in the seed region of the antisense strand of the siRNA provided unexpectedly and advantageously increased gene knockdown activity in vitro.

In vitro transfection was performed in an A549 cell line to determine knockdown efficacy for GST-π siRNAs based on structure BU2′ (SEQ ID NOs:133 and 159). Dose dependent knockdown of GST-πmRNA was observed with GST-π siRNAs based on structure BU2′ as shown in Table 11.

TABLE 11
Dose dependent knockdown of GST-π mRNA in an A549 cell
line for GST-π siRNAs based on structure BU2’

| GST-π siRNA structure | IC50 (pM) |
|---|---|
| BU2 with no deoxynucleotides in the duplex region | 31 |
| (SEQ ID NOs: 63 and 128) | |
| BU2 with deoxynucleotides in positions 3, 5, and 7 of | 5 |
| the seed region antisense strand (SEQ ID NOs: 141 | |
| and 167) | |
| BU2 with deoxynucleotides in positions 4, 6, and 8 of | 8 |
| the seed region antisense strand (SEQ ID NOs: 143 | |
| and 169) | |
| BU2 with deoxynucleotides in positions 4, 6, and 8 of | 5 |
| the seed region antisense strand (SEQ ID NOs: 158 | |
| and 184) | |

As shown in Table 11, the activities of GST-π siRNAs based on structure BU2′ having three deoxynucleotides in the seed region of the antisense strand were surprisingly and unexpectedly increased by up to 6-fold, as compared to a GST-π siRNA without deoxynucleotides in the duplex region.

These data show that GST-π siRNAs having a structure with three deoxynucleotides located at positions 3, 5 and 7, or at positions 4, 6 and 8 in the seed region of the antisense strand provided surprisingly increased gene knockdown activity as compared to a GST-π siRNA without deoxynucleotides in the duplex region.

The activities shown in Table 11 for GST-π siRNAs having three deoxynucleotides in the seed region of the antisense strand were in the range 5 to 8 pM, which is exceptionally suitable for many uses, including as a drug agent to be used in vivo.

Example 3: The structure of GST-π siRNAs of this invention having deoxynucleotides located in the seed region of the anti sense strand of the siRNA provided unexpectedly and advantageously increased gene knockdown activity in vitro.

In vitro transfection was performed in an A549 cell line to determine knockdown efficacy for GST-π siRNAs based on structure A9′ (SEQ ID NOs:185 and 197). Dose dependent knockdown of GST-π mRNA was observed with the GST-π siRNAs based on structure A9′, as shown in Table 12.

TABLE 12
Dose dependent knockdown of GST-π mRNA in an A549 cell
line for GST-π siRNAs based on structure structure A9’

| GST-π siRNA structure | IC50 (pM) |
|---|---|
| A9 with no deoxynucleotides in the duplex region | 24 |
| (SEQ ID NOs: 27 and 92) | |
| A9 with deoxynucleotides in positions 4, 6, and 8 of | 1 |
| the seed region antisense strand (SEQ ID NOs: 195 | |
| and 207) | |
| A9 with deoxynucleotides in positions 1, 3, 5, and 7 | 5 |
| of the seed region antisense strand (SEQ ID NOs: 192 | |
| and 204) | |
| A9 with deoxynucleotides in positions 3-8 of the seed | 6 |
| region antisense strand (SEQ ID NOs: 189 and 201) | |
| A9 with deoxynucleotides in positions 5-8 of the seed | 7 |
| region antisense strand (SEQ ID NOs: 191 and 203) | |
| A9 with deoxynucleotides in positions 3, 5, and 7 of | 15 |
| the seed region antisense strand (SEQ ID NOs: 193 | |
| and 205) | |

As shown in Table 12, the activities of GST-π siRNAs based on structure A9′ having three to six deoxynucleotides in the seed region of the anti sense strand were surprisingly increased by up to 24-fold, as compared to a GST-π siRNA without deoxynucleotides in the duplex region.

These data show that GST-π siRNAs having a structure with three to six deoxynucleotides located at positions 4, 6 and 8, or at positions 1, 3, 5 and 7, or at positions 3-8, or at positions 5-8, or at positions 3, 5 and 7 in the seed region of the antisense strand provided unexpectedly increased gene knockdown activity as compared to a GST-π siRNA without deoxynucleotides in the duplex region.

The activity shown in Table 12 for GST-π siRNAs having three to six deoxynucleotides in the seed region of the antisense strand was in the range 1 to 15 pM, which is exceptionally suitable for many uses, including as a drug agent to be used in vivo.

Example 4: The structure of GST-π siRNAs having deoxynucleotides located in the seed region of the antisense strand of the siRNA provided unexpectedly and advantageously increased gene knockdown activity in vitro.

In vitro transfection was performed in an A549 cell line to determine knockdown efficacy for GST-π siRNAs based on structure B13′ (SEQ ID NOs:209 and 224). Dose dependent knockdown of GST-πmRNA was observed with the GST-π siRNAs based on structure B13′, as shown in Table 13.

TABLE 13
Dose dependent knockdown of GST-π mRNA in an A549 cell
line for GST-π siRNAs based on structure B13’

| GST-π siRNA structure | IC50 (pM) |
|---|---|
| B13 with no deoxynucleotides in the duplex region | 17 |
| (SEQ ID NOs: 52 and 117) | |
| B13 with deoxynucleotides in positions 4, 6, and 8 of | 11 |
| the seed region antisense strand (SEQ ID NOs: 219 | |
| and 234) | |

As shown in Table 13, the activity of a GST-π siRNA based on structure B13′ having three deoxynucleotides in the seed region of the antisense strand was unexpectedly increased, as compared to a GST-π siRNA without deoxynucleotides in the duplex region.

These data show that GST-π siRNAs having a structure with three deoxynucleotides located at positions 4, 6 and 8 in the seed region of the antisense strand provided unexpectedly increased gene knockdown activity as compared to a GST-π siRNA without deoxynucleotides in the duplex region.

The activity shown in Table 13 for GST-π siRNAs having three deoxynucleotides in the seed region of the antisense strand was in the picomolar range at 11 pM, which is exceptionally suitable for many uses, including as a drug agent to be used in vivo.

Example 5: The structure of GST-π siRNAs having deoxynucleotides located in the seed region of the antisense strand of the siRNA provided unexpectedly and advantageously increased gene knockdown activity in vitro.

In vitro transfection was performed in an A549 cell line to determine knockdown efficacy for GST-π siRNAs based on structure B4′ (SEQ ID NOs:263 and 275). Dose dependent knockdown of GST-π mRNA was observed with the GST-π siRNAs based on structure B4′, as shown in Table 14.

TABLE 14
Dose dependent knockdown of GST-π mRNA in an A549 cell
line for GST-π siRNAs based on structure B4’

| GST-π siRNA structure | IC50 (pM) |
|---|---|
| B4 with no deoxynucleotides in the duplex region | 229 |
| (SEQ ID NOs: 56 and 121) | |
| B4 with deoxynucleotides in positions 3-8 of the seed | 113 |
| region antisense strand (SEQ ID NOs: 267 and 279) | |

As shown in Table 14, the activities of GST-π siRNAs based on structure B4′ having six deoxynucleotides in the seed region of the antisense strand were unexpectedly increased by more than two-fold, as compared to a GST-π siRNA without deoxynucleotides in the duplex region.

These data show that GST-π siRNAs having a structure with six deoxynucleotides located at positions 3-8 in the seed region of the antisense strand provided surprisingly increased gene knockdown activity as compared to a GST-π siRNA without deoxynucleotides in the duplex region.

The activity shown in Table 14 for a GST-π siRNA having six deoxynucleotides in the seed region of the antisense strand was in the picomolar range at 113 pM, which is exceptionally suitable for many uses, including as a drug agent to be used in vivo.

Example 6: The structure of GST-π siRNAs having deoxynucleotides located in the seed region of the antisense strand of the siRNA provided unexpectedly and advantageously increased gene knockdown activity in vitro.

In vitro transfection was performed in an A549 cell line to determine knockdown efficacy for GST-π siRNAs based on structure B2′ (SEQ ID NOs:239 and 251). Dose dependent knockdown of GST-πmRNA was observed with the GST-π siRNAs based on structure B2′, as shown in Table 15.

TABLE 15
Dose dependent knockdown of GST-π mRNA in an A549 cell
line for GST-π siRNAs based on structure B2’

| GST-π siRNA structure | IC50 (pM) |
|---|---|
| B2 with no deoxynucleotides in the duplex regioin | 121 |
| (SEQ ID NOs: 54 and 119) | |
| B2 with deoxynucleotides in positions 5-8 of the seed | 30 |
| region antisense strand (SEQ ID NOs: 245 and 257) | |
| B2 with deoxy nucleotides in positions 1, 3, 5, and 7 | 50 |
| of the seed region antisense strand (SEQ ID NOs: 246 | |
| and 258) | |
| B2 with deoxy nucleotides in positions 3, 5, and 7 of | 100 |
| the seed region antisense strand (SEQ ID NOs: 246 | |
| and 259) | |

As shown in Table 15, the activities of GST-π siRNAs based on structure B2′ having three to four deoxynucleotides in the seed region of the antisense strand were surprisingly increased by up to 4-fold, as compared to a GST-π siRNA without deoxynucleotides in the duplex region.

These data show that GST-π siRNAs having a structure with three to four deoxynucleotides located at positions 5-8, or at positions 1, 3, 5 and 7, or at positions 3, 5 and 7 in the seed region of the antisense strand provided unexpectedly increased gene knockdown activity as compared to a GST-π siRNA without deoxynucleotides in the duplex region.

The activities shown in Table 15 for GST-π siRNAs having three to four deoxynucleotides in the seed region of the anti sense strand were in the range 30-100 pM, which is exceptionally suitable for many uses, including as a drug agent to be used in vivo.

Example 7: The structure of GST-π siRNAs containing one or more 2′-deoxy-2′-fluoro substituted nucleotides provided unexpectedly increased gene knockdown activity in vitro.

In vitro transfection was performed in an A549 cell line to determine knockdown efficacy for GST-π siRNAs based on structure BU2′ (SEQ ID NOs:133 and 159). Dose dependent knockdown of GST-π mRNA was observed with the GST-π siRNAs based on structure BU2′, as shown in Table 16.

TABLE 16
Dose dependent knockdown of GST-π mRNA in an A549 cell
line for GST-π siRNAs based on structure BU2’

| GST-π siRNA structure | IC50 (pM) |
|---|---|
| BU2 with no 2’-F deoxynucleotides | 31 |
| (SEQ ID NOs: 63 and 128) | |
| BU2 with seven 2’-F deoxynucleotides, one in | 3 |
| position 1 at the 3’end of the antisense strand (SEQ | |
| ID NOs: 150 and 176) | |
| BU2 with four 2’-F deoxynucleotides, one in position | 11 |
| 1 at the 3’end of the antisense strand (SEQ ID | |
| NOs: 149 and 175) | |
| BU2 with one 2’-F deoxynucleotide in position 1 at | 13 |
| the 3’end of the antisense strand (SEQ ID NOs: 146 | |
| and 172) | |

As shown in Table 16, the activities of GST-π siRNAs based on structure BU2′ having one or more 2′-F deoxynucleotides were surprisingly increased by up to 10-fold, as compared to a GST-π siRNA without 2′-F deoxynucleotides.

These data show that GST-π siRNAs having a structure with one or more 2′-F deoxynucleotides provided unexpectedly increased gene knockdown activity as compared to a GST-π siRNA without a 2′-F deoxynucleotide.

The activities shown in Table 16 for GST-π siRNAs having one or more deoxynucleotides were in the range 3 to 13 pM, which is exceptionally suitable for many uses, including as a drug agent to be used in vivo.

Example 8: The structure of GST-π siRNAs containing one or more 2′-deoxy-2′-fluoro substituted nucleotides provided unexpectedly increased gene knockdown activity in vitro.

In vitro transfection was performed in an A549 cell line to determine knockdown efficacy for GST-π siRNAs based on structure B13′ (SEQ ID NOs:209 and 224). Dose dependent knockdown of GST-πmRNA was observed with the GST-πsiRNAs based on structure B13′, as shown in Table 17.

TABLE 17
Dose dependent knockdown of GST-π mRNA in an A549 cell
line for GST-π siRNAs based on structure B13’

| GST-π siRNA structure | IC50 (pM) |
|---|---|
| B13 with no 2’-F deoxynucleotides | 17 |
| (SEQ ID NOs: 52 and 117) | |
| B13 with three 2’-F deoxynucleotides located in non- | 6 |
| overhang positions (SEQ ID NOs: 221 and 236) | |

As shown in Table 17, the activity of a GST-π siRNA based on structure B13′ having three 2′-F deoxynucleotides located in non-overhang positions was surprisingly increased by about 3-fold, as compared to a GST-π siRNA without 2′-F deoxynucleotides.

These data show that GST-π siRNAs having a structure with one or more 2′-F deoxynucleotides provided unexpectedly increased gene knockdown activity as compared to a GST-π siRNA without a 2′-F deoxynucleotide.

The activity shown in Table 17 for GST-π siRNAs having one or more 2′-F deoxynucleotides was in the picomolar range at 6 pM, which is exceptionally suitable for many uses, including as a drug agent to be used in vivo.

Example 9: Orthotopic A549 lung cancer mouse model. The GST-π siRNAs of this invention can exhibit profound reduction of orthotopic lung cancer tumors in vivo. In this example, a GST-π siRNA provided gene knockdown potency in vivo when administered in a liposomal formulation to the orthotopic lung cancer tumors in athymic nude mice.

In general, an orthotopic tumor model can exhibit direct clinical relevance for drug efficacy and potency, as well as improved predictive ability. In the orthotopic tumor model, tumor cells are implanted directly into the same kind of organ from which the cells originated.

The anti-tumor efficacy of the siRNA formulation against human lung cancer A549 was evaluated by comparing the final primary tumor weights measured at necropsy for the treatment group and the vehicle control group.

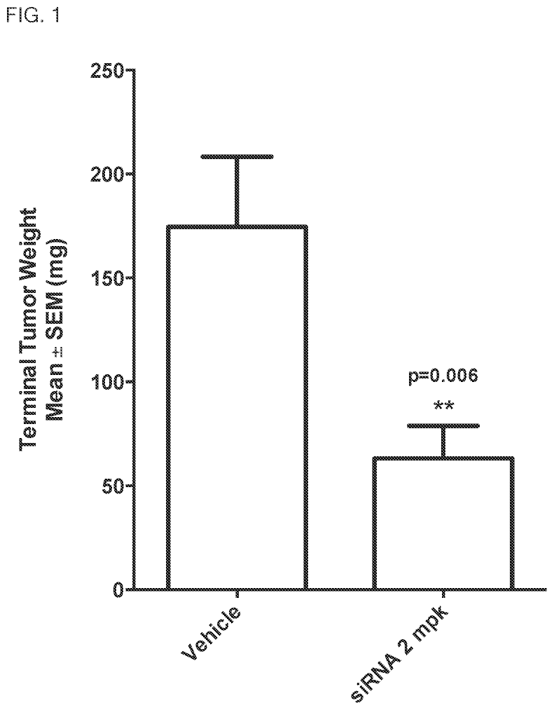

FIG. 1 shows orthotopic lung cancer tumor inhibition in vivo for a GST-π siRNA based on structure BU2 (SEQ ID NOs:63 and 128). An orthotopic A549 lung cancer mouse model was utilized with a relatively low dose at 2 mg/kg of the siRNA targeted to GST-π.

The GST-π siRNA showed significant and unexpectedly advantageous lung tumor inhibition efficacy in this six-week study. As shown in FIG. 1, after 43 days, the GST-π siRNA showed markedly advantageous tumor inhibition efficacy, with final tumor average weights significantly reduced by 2.8-fold as compared to control.

For this study, male NCr nu/nu mice, 5-6 weeks old, were used. The experimental animals were maintained in a HEPA filtered environment during the experimental period. The siRNA formulations were stored at 4° C. before use, and warmed to room temperature 10 minutes prior to injection in mouse.

For this A549 human lung cancer orthotopic model, on the day of surgical orthotopic implantation (SOI), the stock tumors were harvested from the subcutaneous site of animals bearing A549 tumor xenograft and placed in RPMI-1640 medium. Necrotic tissues were removed and viable tissues were cut into 1.5-2 mm³ pieces. The animals were anesthetized with isoflurane inhalation and the surgical area was sterilized with iodine and alcohol. A transverse incision approximately 1.5 cm long was made in the left chest wall of the mouse using a pair of surgical scissors. An intercostal incision was made between the third and the fourth rib and the left lung was exposed. One A549 tumor fragment was transplanted to the surface of the lung with an 8-0 surgical suture (nylon). The chest wall was closed with a 6-0 surgical suture (silk). The lung was re-inflated by intrathoracic puncture using a 3 cc syringe with a 25 G×1½ needle to draw out the remaining air in the chest cavity. The chest wall was closed with a 6-0 surgical silk suture. All procedures of the operation described above were performed with a 7× magnification microscope under HEPA filtered laminar flow hoods.

Three days after tumor implantation, the model tumor-bearing mice were randomly divided into groups of ten mice per group. For the group of interest, treatment of the ten mice was initiated three days after tumor implantation.

For the group of interest, the formulation was (Ionizable lipid:cholesterol:DOPE:DOPC:DPPE-PEG-2K:DSPE-PEG-2K), a liposomal composition. The liposomes encapsulated the GST-π siRNA.

For the study endpoint, the experimental mice were sacrificed forty-two days after treatment initiation. Primary tumors were excised and weighed on an electronic balance for subsequent analysis.

For an estimation of compound toxicity, the mean body weight of the mice in the treated and control groups was maintained within the normal range during the entire experimental period. Other symptoms of toxicity were not observed in the mice.

Example 10: The GST-π siRNAs of this invention exhibited profound reduction of cancer xenograft tumors in vivo. The GST-π siRNAs provided gene knockdown potency in vivo when administered in a liposomal formulation to the cancer xenograft tumors.

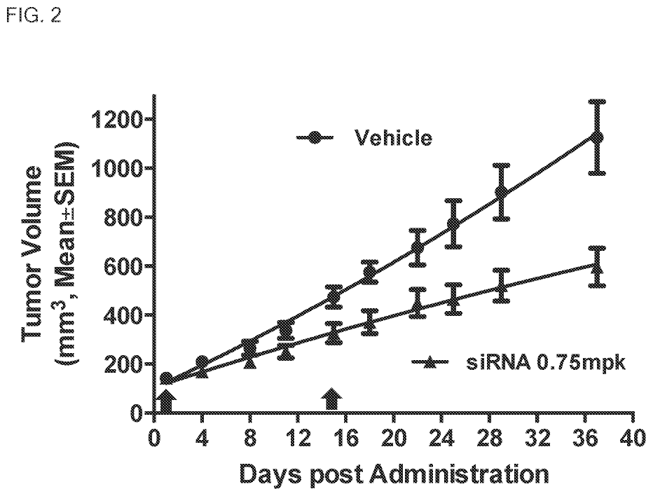

FIG. 2 shows tumor inhibition efficacy for a GST-π siRNA (SEQ ID Nos:158 and 184). A cancer xenograft model was utilized with a relatively low dose at 0.75 mg/kg of siRNA targeted to GST-π.

The GST-π siRNA showed significant and unexpectedly advantageous tumor inhibition efficacy within a few days after administration. After 36 days, the GST-π siRNA showed markedly advantageous tumor inhibition efficacy, with tumor volume reduced by 2-fold as compared to control.

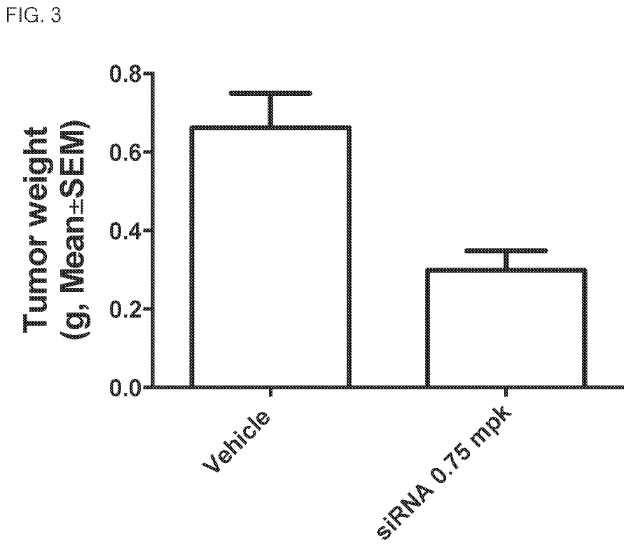

As shown in FIG. 3, the GST-π siRNA demonstrated significant and unexpectedly advantageous tumor inhibition efficacy at the endpoint day. In particular, tumor weight was reduced by more than 2-fold.

The GST-π siRNA was administered in two injections (day 1 and 15) of a liposomal formulation having the composition (Ionizable lipid:Cholesterol:DOPE:DOPC:DPPE-PEG-2K) (25:30:20:20:5).

For the cancer xenograft model, an A549 cell line was obtained from ATCC. The cells were maintained in culture medium supplemented with 10% Fetal Bovine Serum and 100 U/ml penicillin and 100 μg/ml streptomycin. Cells were split 48 hrs before inoculation so that cells were in log phase growth when harvested. Cells were trypsinized with trypsin-EDTA and harvested from tissue culture. The number of viable cells was counted and determined in a hemocytometer in the presence of trypan blue (only viable cells are counted). The cells were resuspended to a concentration of 5×10⁷/ml in media without serum. Then the cell suspension was mixed well with ice thawed BD matrigel at 1:1 ratio for injection.

Mice were Charles River Laboratory Athymic Nude (nu/nu) Female Mice, immuno-compromised, 6-8 weeks old, 7-8 mice per group.

For tumor model preparation, each mouse was inoculated subcutaneously in the right flank with 0.1 ml an inoculum of 2.5×10⁶ of A549 cells using a 25 G needle and syringe, one inoculum per mouse. Mice were not anesthetized for inoculation.

For tumor volume measurements and randomization, tumor size was measured to the nearest 0.1 mm. Tumor volumes were calculated using the formula: Tumor volume=length×width²/2. Once the established tumors reached approximately 120-175 mm³, average tumor volume was about 150 mm³, the mice were assigned into the various vehicle control and treatment groups such that the mean tumor volumes in the treated groups were within 10% of the mean tumor volume in the vehicle control group, ideally, the CV % of tumor volume was less than 25%. On the same day, test articles and control vehicle were administered according to the dosing regimen. Tumor volumes were monitored three times for week 1, twice for the rest of weeks, including the day of study termination.

For dosage administration, on the dosing day, the test articles were taken out from −80° C. freezer and thawed on ice. Before applied to syringes, the bottle containing formulation was reverted by hands for a few times. All test articles were dosed at 0.75 mg/kg by IV, q2w×2, at 10 ml/kg.

For body weight, mice were weighed to the nearest 0.1 g. Body weights were monitored and recorded daily within 7 days post dosing for first dose. Body weights were monitored and recorded twice for weeks, for the rest of weeks, including the day of study termination.

For tumors collection, on 28 days post first dosing, tumor volume was measured, and tumor was dissected for weight measurement, and stored for PD biomarker study. Tumor weight was recorded.

Example 11: The GST-π siRNAs of this invention demonstrated increased cancer cell death by apoptosis of cancer cells in vitro. The GST-π siRNAs provided GST-π knockdown, which resulted in upregulation of PUMA, a biomarker for apoptosis and associated with loss in cell viability.

GST-π siRNA SEQ NOs:158 and 184, which contained a combination of deoxynucleotides in the seed region, a 2′-F substituted deoxynucleotide, and 2′-OMe substituted ribonucleotides, provided unexpectedly increased apoptosis of cancer cells.

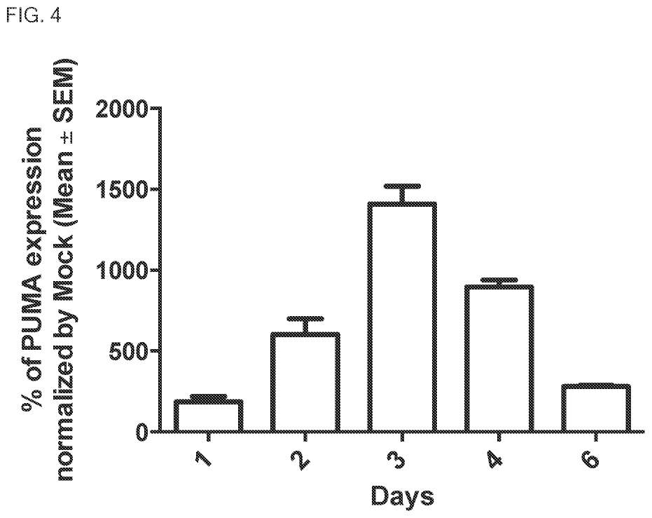

The level of expression of PUMA for GST-π siRNA SEQ ID NOs:158 and 184 was measured as shown in FIG. 4. In FIG. 4, the expression of PUMA was greatly increased from 2-4 days after transfection of the GST-π siRNA.

These data show that the structure of GST-π siRNAs containing a combination of deoxynucleotides in the seed region, a 2′-F substituted deoxynucleotide, and 2′-OMe substituted ribonucleotides provided unexpectedly increased apoptosis of cancer cells.

The protocol for the PUMA biomarker was as follows. One day before transfection, cells were plated in a 96-well plate at 2×10³ cells per well with 100 μl of DMEM (HyClone Cat. #S1130243.01) containing 10% FBS and cultured in a 37° C. incubator containing a humidified atmosphere of 5% CO2 in air. Next day, before transfection the medium was replaced with 90 μl of Opti-MEM I Reduced Serum Medium (Life Technologies Cat. #31985-070) containing 2% FBS. Then, 0.2 μl of Lipofectamine RNAiMAX (Life Technologies Cat. #13778-100) were mixed with 4.8 of Opti-MEM I for 0.5 minutes at room temperature. 1 μl of the GST-π siRNA (stock conc. 1 μM) was mixed with 4 μl of Opti-MEM I and combined with the RNAiMAX solution and then mixed gently. The mixture was incubated for 10 minutes at room temperature to allow the RNA-RNAiMAX complexes to form, 10 μl of RNA-RNAiMAX complexes were added per well, to final concentration of the siRNA 10 nM. The cells were incubated for 2 hours and medium changed to fresh Opti-MEM I Reduced Serum Medium containing 2% FBS. For 1, 2, 3, 4, and 6 days post transfection, the cells were washed with ice-cold PBS once and then lysed with 50 μl of Cell-to-Ct Lysis Buffer (Life Technologies Cat. #4391851 C) for 5-30 minutes at room temperature. 5 μl of Stop Solution was added and incubated for 2 minutes at room temperature. PUMA (BBC3, Cat #Hs00248075, Life Technologies) mRNA levels were measured by qPCR with TAQMAN.

Example 12: The GST-π siRNAs of this invention can exhibit profound reduction of cancer xenograft tumors in vivo. The GST-π siRNAs can provide gene knockdown potency in vivo when administered in a liposomal formulation to the cancer xenograft tumors.

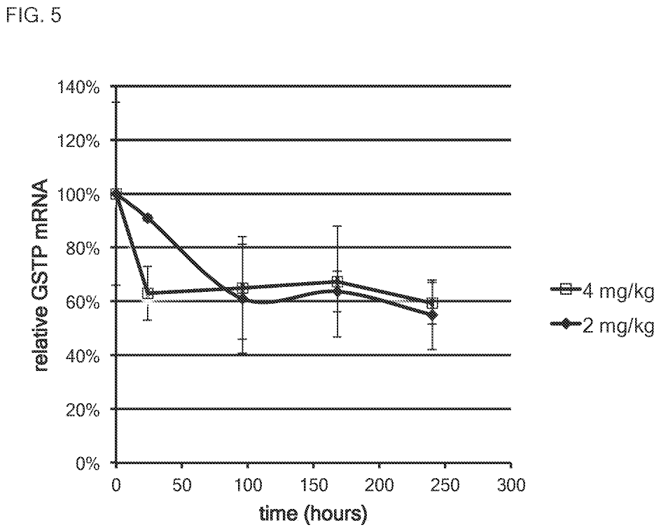

FIG. 5 shows tumor inhibition efficacy for a GST-π siRNA (SEQ ID NOs:63 and 128). Dose dependent knockdown of GST-π mRNA was observed in vivo with the siRNA targeted to GST-π. A cancer xenograft model was utilized with a relatively low dose at 0.75 mg/kg of siRNA targeted to GST-π.

The GST-π siRNA showed significant and unexpectedly advantageous tumor inhibition efficacy within a few days after administration. As shown in FIG. 5, treatment with a GST-π siRNA resulted in significant reduction of GST-π mRNA expression 4 days after injection in a lipid formulation. At the higher dose of 4 mg/kg, significant reduction of about 40% was detected 24 hours after injection.

The GST-π siRNA was administered in a single injection of 10 mL/kg of a liposomal formulation having the composition (Ionizable lipid:Cholesterol:DOPE:DOPC:DPPE-PEG-2K) (25:30:20:20:5).

For the cancer xenograft model, an A549 cell line was obtained from ATCC. The cells were maintained in RPMI-1640 supplemented with 10% Fetal Bovine Serum and 100 U/ml penicillin and 100 μg/ml streptomycin. Cells were split 48 hrs before inoculation so that cells were in log phase growth when harvested. Cells were lightly trypsinized with trypsin-EDTA and harvested from tissue culture. The number of viable cells was counted and determined in a hemocytometer in the presence of trypan blue (only viable cells are counted). The cells were resuspended to a concentration of 4×10⁷/ml in PMI media without serum. Then the cell suspension was mixed well with ice thawed BD matrigel at 1:1 ratio for injection.

Mice were Charles River Laboratory Athymic Nude (nu/nu) Female Mice, immuno-compromised, 6-8 weeks old, 3 mice per group.

For tumor model preparation, each mouse was inoculated subcutaneously in the right flank with 0.1 ml an inoculum of 2×10⁶ of A549 cells using a 25 G needle and syringe, one inoculum per mouse. Mice were not anesthetized for inoculation.

For tumor volume measurements and randomization, tumor size was measured to the nearest 0.1 mm. Tumor volumes were calculated using the formula: Tumor volume=length×width²/2. Tumor volumes were monitored twice a week. Once the established tumors reached approximately 350-600 mm³, the mice were assigned into groups with varied time points. On the same day, test articles were administered according to the dosing regimen.

For dosage administration, on the day when the established tumors reached approximately 350-600 mm³, the test articles were taken out from 4° C. fridge. Before being applied to syringes, the bottle containing formulation was reverted by hand for a few times to make a homogeneous solution.

For body weight, mice were weighed to the nearest 0.1 g. Body weights were monitored and recorded twice for weeks, for the rest of weeks, including the day of study termination.

For tumors collection, animals were sacrificed by overdosed CO₂ and tumors were dissected at 0, 24, 48, 72, 96 (optional), and 168 hours following the dosing. Tumors were first wet weighted, and then separated into three parts for KD, distribution and biomarker analysis. The samples were snap frozen in liquid nitrogen and stored at −80° C. until ready to be processed.

Example 13: The GST-π siRNAs of this invention inhibited pancreatic cancer xenograft tumors in vivo. The GST-π, siRNAs provided gene knockdown potency in vivo when administered in a liposomal formulation to the pancreatic cancer xenograft tumors.

In this xenograft model, each mouse was inoculated subcutaneously in the right flank with 0.1 ml an inoculum of 2.5×10⁶ of PANC-1 cells. Athymic nude female mice, 6 to 8 weeks, Charles River, were used. Tumor size was measured to the nearest 0.1 mm. Once the established tumors reached approximately 150-250 mm³ (average tumor volume at about 200 mm³), the mice were assigned into the various vehicle control and treatment groups such that the mean tumor volumes in the treated groups were within 10% of the mean tumor volume in the vehicle control group. On the same day, test articles and control vehicle were administered according to the dosing regimen. Tumor volumes were monitored three times for week 1, twice for the rest of weeks, including the day of study termination.

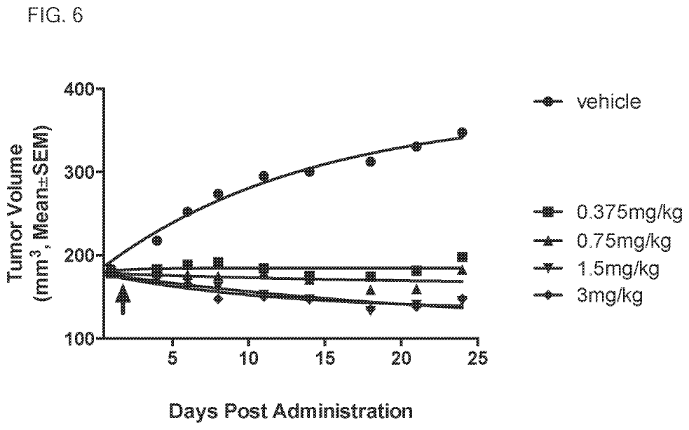

FIG. 6 shows tumor inhibition efficacy for a GST-π siRNA (SEQ ID Nos:63 and 128). As shown in FIG. 6, a dose response was obtained with doses ranging from 0.375 mg/kg to 3 mg/kg of siRNA targeted to GST-π. The GST-π siRNA showed significant and unexpectedly advantageous tumor inhibition efficacy within a few days after administration. Thus, the GST-π siRNA demonstrated significant and unexpectedly advantageous tumor inhibition efficacy at the endpoint.

The GST-π siRNAs were administered in a liposomal formulation having the composition (Ionizable lipid:cholesterol:DOPE:DOPC:DPPE-PEG-2K) (25:30:20:20:5).

Example 14: The GST-π siRNAs of this invention exhibited increased serum stability.

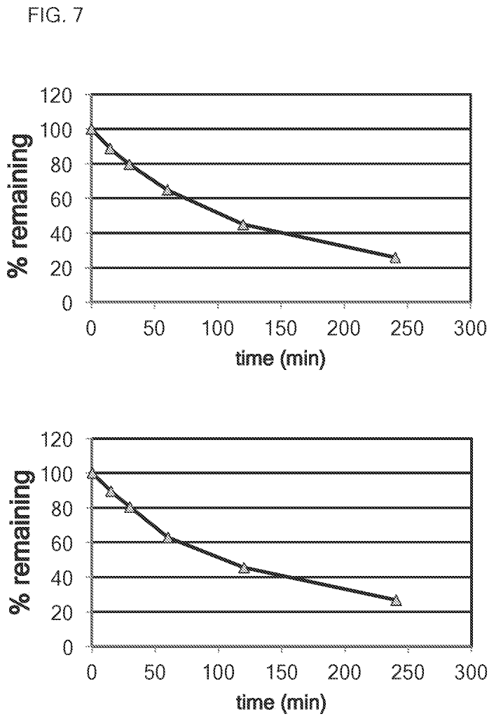

FIG. 7 shows incubation in human serum and detection of remaining siRNA at various time points by HPLS/LCMS. As shown in FIG. 7, the half-life (t_{1/2}) in serum for both the sense strand (FIG. 7, top) and antisense strand (FIG. 7, bottom) of a GST-π siRNA (SEQ ID Nos:63 and 128) was about 100 minutes.

Example 15: The GST-π siRNAs of this invention exhibited enhanced stability in formulation in plasma.

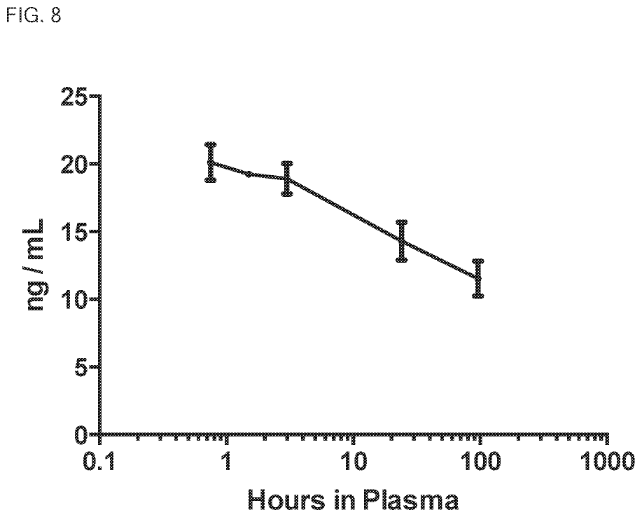

FIG. 8 shows incubation of formulation in plasma and detection of remaining siRNA at various time points. As shown in FIG. 8, the half-life (t_{1/2}) in plasma of a formulation of GST-π siRNA (SEQ ID Nos:63 and 128) was significantly longer than 100 hours.

The GST-π siRNA was prepared in a liposomal formulation having the composition (Ionizing lipid:cholesterol:DOPE:DOPC:DPPE-PEG-2K) (25:30:20:20:5), The z-average size for the liposomal nanoparticles was 40.0 nm, and the siRNA was 91% encapsulated.

The formulation was incubated in 50% human serum in PBS for 40 min, 1.5 h, 3 h, 24 h, and 96 h. The amount of the GST-π siRNA was determined by an ELISA-based assay.

Example 16: The GST-π siRNAs of this invention exhibited reduced off target effects by the passenger strand.

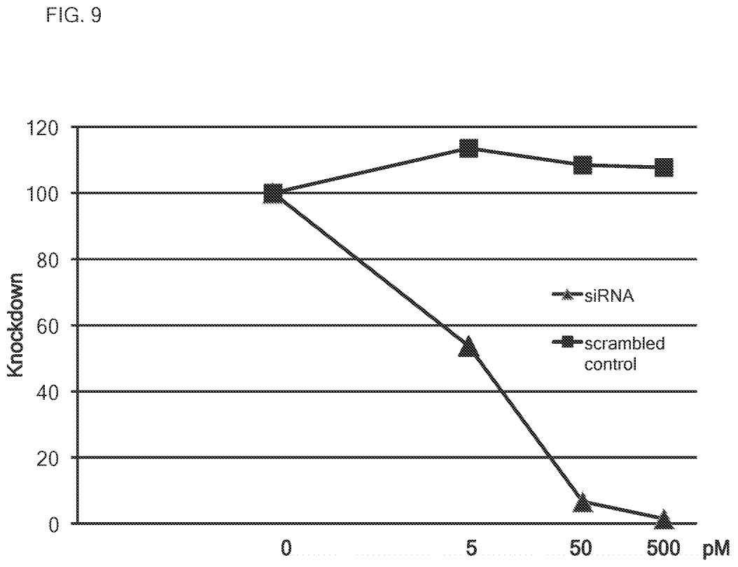

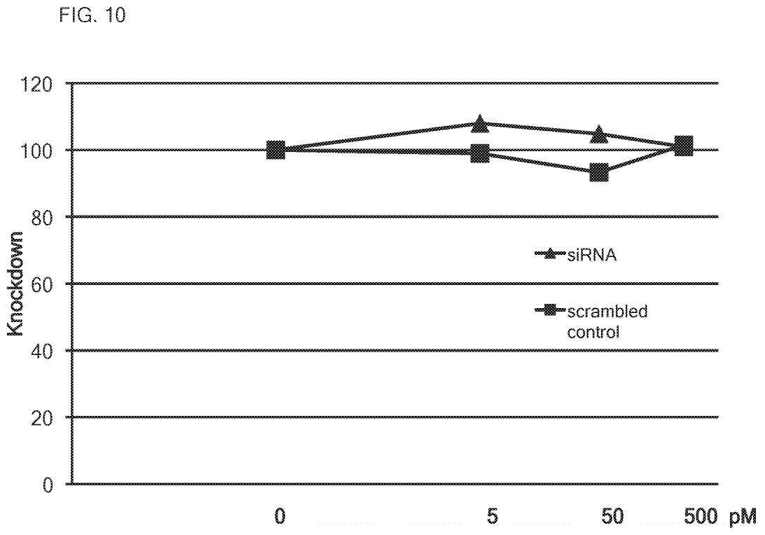

For the GST-π siRNA (SEQ ID Nos:158 and 184), FIG. 9 shows that in vitro knockdown for the guide strand was approximately exponential, as compared to a control with scrambled sequence that exhibited no effect. The IC50 of this siRNA was measured at 5 pM. FIG. 10 shows in vitro knockdown for the passenger strand of the same GST-π siRNA. As shown in FIG. 10, the passenger strand off target knockdown for the GST-π siRNA was greatly reduced, by more than 100-fold.

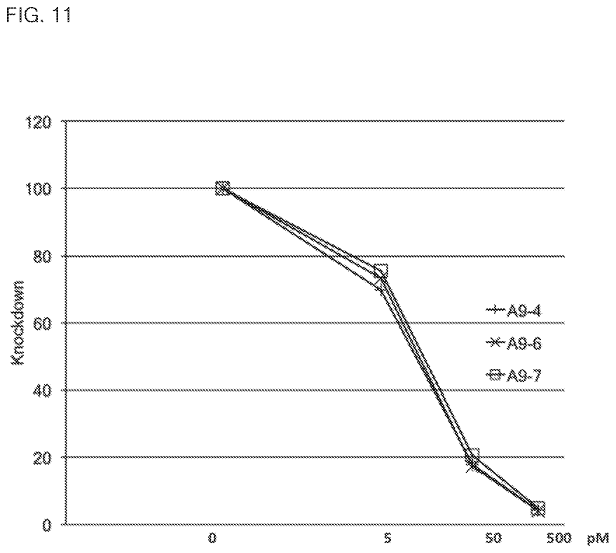

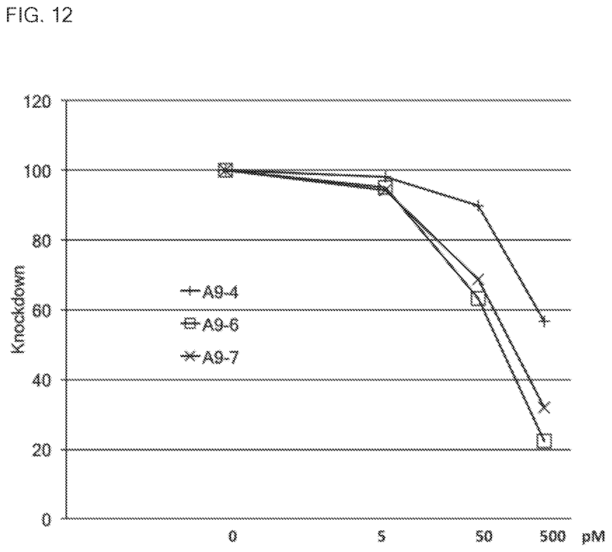

For the GST-π siRNAs (SEQ ID Nos:189 and 201), (SEQ ID Nos:191 and 203), and (SEQ ID Nos:192 and 204), FIG. 11 shows that the in vitro knockdowns for the guide strands were approximately exponential. The IC50s of these siRNAs were measured at 6, 7, and 5 pM, respectively. As shown in FIG. 12, the in vitro knockdowns for the passenger strands of these GST-π siRNAs were significantly reduced by at least 10-fold. All of these GST-π siRNAs had deoxynucleotides in the seed region of the duplex region, with no other modifications in the duplex region.

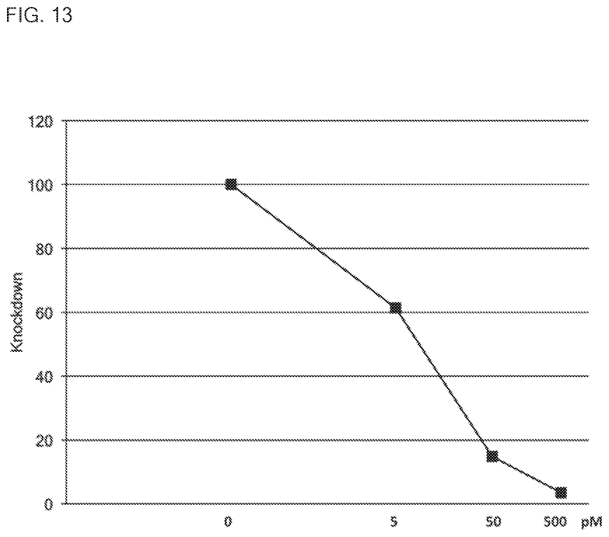

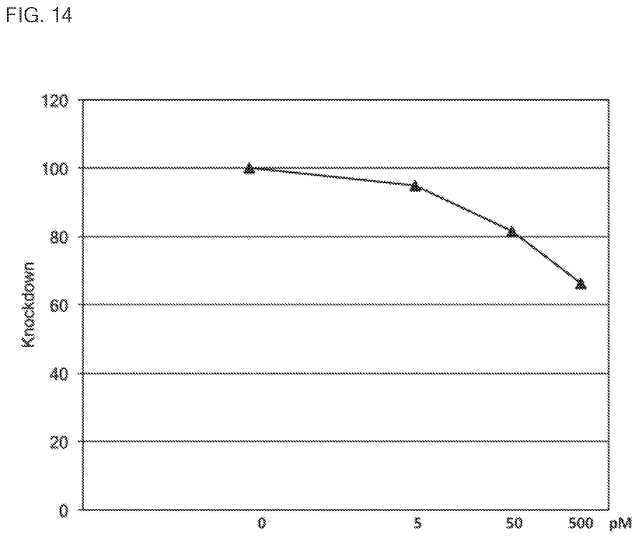

For the GST-π siRNAs (SEQ ID Nos:219 and 234), FIG. 13 shows that the in vitro knockdown for the guide strand of this highly active GST-π siRNA was approximately exponential. The IC50 of this siRNA was measured at 11 pM. As shown in FIG. 14, the in vitro knockdown for the passenger strand of this GST-π siRNA was significantly reduced by more than 100-fold. This GST-π siRNA had deoxynucleotides in the seed region of the duplex region, with no other modifications in the duplex region.

Off-target effects were determined using the expression reporter plasmid psiCHECK-2, which encodes the Renilla luciferase gene. (Dual-Luciferase Reporter Assay System, Promega, Cat #:E1960). The siRNA concentration was typically 50 pM. Protocol: Day 1, HeLa cell seeded at 5 to 7.5×103/100 ul/well. Day 2, co-transfection with cell confluence about 80%. Day 3, cells harvested for luciferase activity measurement. Luciferase activity was measured using Promega's Luciferase Assay System (E4550), according to manufacturer's protocol.

The psiCHECK-2 vector enabled monitoring of changes in expression of a target gene fused to the reporter gene of Renilla luciferase. The siRNA constructs were cloned into the multiple cloning region, and the vector was cotransfected with the siRNA into HeLa cells. If a specific siRNA binds to the target mRNA and initiates the RNAi process, the fused Renilla luciferase: construct mRNA will be cleaved and subsequently degraded, decreasing the Renilla luciferase signal.

For example, the plasmid inserts for siRNAs the BU2′ structure were as follows:

PsiCHECK-2 (F) plasmid insert:
SEQ ID NO.: 288
ctcgag gggcaacTGAAGCCTTTTGAGACCCTGcTgTcccag
gcggccgc
PsiCHECK-2 (R) plasmid insert:
SEQ ID NO.: 289
ctcgag cTgggacagCAGGGTCTCAAAAGGCTTCagTTgccc
gcggccgc

Example 17: The GST-π siRNAs of this invention exhibited advantageously reduced miRNA-like off target effects, which are seed-dependent unintended off-target gene silencing.

For the GST-π siRNAs (SEQ ID Nos:158 and 184), (SEQ ID Nos:189 and 201), (SEQ ID Nos:191 and 203), (SEQ Nos:192 and 204), and (SEQ ID Nos:219 and 234), off target activity mimicking miRNA was found to be essentially negligible. The seed-dependent unintended off-target gene silencing for these GST-π siRNAs was at least 10-fold to 100-fold less than the on-target activity of the guide strand.

For testing miRNA-related off target effects, one to four repeats of seed-matched target sequences complementary to the entire seed-containing region, positions 1-8 of the 5′ end of the antisense strand, but not to the remaining non-seed region, positions 9-21, were introduced into the region corresponding to the 3′UTR of the luciferase mRNA, to determine the efficiency of the seed-dependent unintended off-target effects. Plasmid inserts were used to mimic a miRNA with complete matching in the seed region and mismatches (bulges) in the non-seed region.

For example, the plasmid inserts for siRNAs with the BU2′ structure were as follows:

PsiCHECK-2 (Fmi1) plasmid insert:
SEQ ID NO.: 290
ctcgag gggcaacTCTACGCAAAACAGACCCTGcTgTcccag
gcggccgc
PsiCHECK-2 (Fmi2) plasmid insert:
SEQ ID NO.: 291
ctcgag gggcaacTCTACGCAAAACAGACCCTGcT
CTACGCAAAACAGACCCTGcT gTcccag gcggccgc
PsiCHECK-2 (Fmi3) plasmid insert:
SEQ ID NO.: 292
ctcgag gggcaacTCTACGCAAAACAGACCCTGcT
CTACGCAAAACAGACCCTGcT
CTACGCAAAACAGACCCTGcT gTcccag gcggccgc
PsiCHECK-2 (Fmi4) plasmid insert:
SEQ ID NO.: 293
ctcgag gggcaacTCTACGCAAAACAGACCCTGcT
CTACGCAAAACAGACCCTGcT CTACGCAAAACAGACCCTGcT
CTACGCAAAACAGACCCTGcT gTcccag gcggccgc

Example 18: Examples of RNAi molecules of this invention targeted to GST-π mRNA are shown in Table 18.

TABLE 18
RNAi molecule sequences for GST-π

| | | SENSE STRAND | | |
|---|---|---|---|---|
| | SEQ | (5′-->3′) | SEQ | ANTISENSE STRAND |
| ID | ID | SEQ ID NOS: | ID | (5′-->3′) |
| (A9) | NO | 294 to 297 | NO | SEQ ID NOS: 298 to 301 |
| 21 | 294 | CCUUUUGAGACC | 298 | ACAgCaGgGUCUCAAAAGGUU |
| | | CUGCUGUUU | | |
| 22 | 295 | CCUUUUGAGACC | 299 | ACAgCaGgGUCUCAAAAGGUU |
| | | CUGCUGUUU | | |
| 23 | 296 | CCUUUUGAGACC | 300 | ACAgCaGgGUCUCAAAAGGUU |
| | | CUGCUGUUU | | |
| 24 | 297 | CCUUUUGAGACC | 301 | ACAgCaGgGUCUCAAAAGGUU |
| | | CUGCUGUUU | | |

Key for Table 18: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U.

The structure of GST-π siRNAs of Table 18 provided unexpectedly increased gene knockdown activity in vitro. In vitro transfection was performed in an A549 cell line to determine knockdown efficacy for GST-π siRNAs of Table 18, which are based on structure A9. Dose dependent knockdown of GST-π mRNA was observed with the GST-π siRNAs of Table 18, as shown in Table 19.

TABLE 19
Dose dependent knockdown of GST-π mRNA in an A549 cell
line for GST-π siRNAs based on structure B13’

| GST-π siRNA structure | IC50 (pM) |
|---|---|
| A9 (SEQ ID NOs: 294 and 298) | 4 |
| A9 (SEQ ID NOs: 295 and 299) | 28 |
| A9 (SEQ ID NOs: 296 and 300) | 12 |
| A9 (SEQ ID NOs: 297 and 301) | 7 |

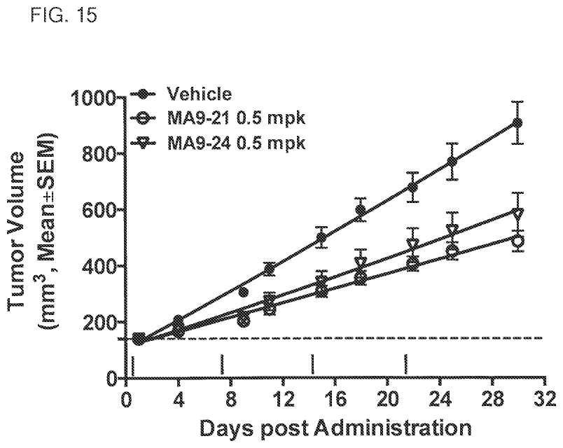

The structure of GST-π siRNAs of Table 18 provided unexpectedly increased tumor inhibition efficacy in vivo, FIG. 15 shows tumor inhibition efficacy in vivo for GST-π siRNAs having structure based on siRNA A9: (SEQ ID NOs:294 and 298) and (SEQ ID NOs:297 and 301). A cancer xenograft model using A549 cells was utilized with a relatively low dose of siRNA at 0.5 mg/kg. The GST-π siRNAs showed advantageous tumor inhibition within a few days. After 36 days, the GST-π siRNAs showed markedly advantageous tumor inhibition, with final tumor average volumes significantly reduced by about 2-fold, as compared to control.

Example 19: Examples of RNAi molecules of this invention targeted to GST-π mRNA are shown in Table 20.

TABLE 20
RNAi molecule sequences for GST-π

| | | SENSE STRAND | | ANTISENSE STRAND |
|---|---|---|---|---|
| | SEQ | (5′-->3′) | SEQ | (5′-->3′) |
| ID | ID | SEQ ID NOS: | ID | SEQ ID NOS: |
| (B13) | NO | 302 to 303 | NO | 304 to 305 |
| 21 | 302 | GGAUGACUAUG | 304 | UGCcUuCaCAUAGUCA |
| | | UGAAGGCAUU | | UCCUU |
| 22 | 303 | GGAUGACUAUG | 305 | UGCcUuCaCAUAGUCA |
| | | UGAAGGCAUU | | UCCUU |

Key for Table 20: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U.

The structure of GST-π siRNAs of Table 18 provided unexpectedly increased gene knockdown activity in vitro. In vitro transfection was performed in an A549 cell line to determine knockdown efficacy for GST-π siRNAs of Table 20, which are based on structure B13. Dose dependent knockdown of GST-πmRNA was observed with the GST-π siRNAs of Table 20, as shown in Table 21.

TABLE 21
Dose dependent knockdown of GST-π mRNA in an A549 cell
line for GST-π siRNAs based on structure B13’

| GST-π siRNA structure | IC50 (pM) |
|---|---|
| B13 (SEQ ID NOs: 302 and 304) | 6 |
| B13 (SEQ ID NOs: 303 and 305) | 5 |

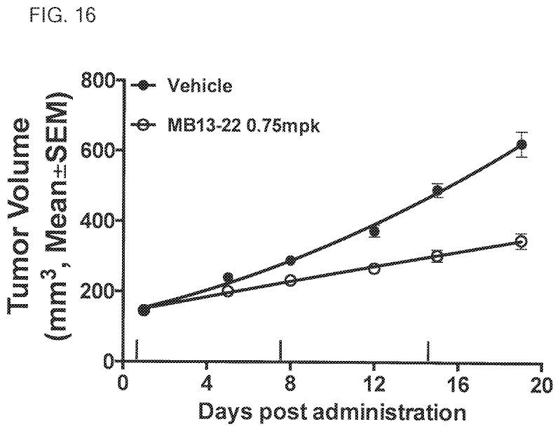

The structure of GST-π siRNAs of Table 20 provided unexpectedly increased tumor inhibition efficacy in vivo. FIG. 16 shows tumor inhibition efficacy in vivo for a GST-π siRNA having structure based on siRNA B13: (SEQ ID NOs:303 and 305). A cancer xenograft model using A549 cells was utilized with a relatively low dose of siRNA at 0.75 mg/kg in a formulation with HEPES buffer. The GST-π siRNA showed advantageous tumor inhibition within a few days. After 36 days, the GST-π siRNA showed markedly advantageous tumor inhibition, with final tumor average volumes significantly reduced by about 2-fold, as compared to control.

### Additional Definitions

The terms used in this specification generally have their ordinary meanings in the art, within the context of the invention, and in the specific context where each term is used, and no special significance is to be placed upon whether or not a term is elaborated upon, or discussed herein. The descriptions of examples in this disclosure are illustrative only, and in no way limit the scope and meaning of the invention.

Unless otherwise defined, all technical and scientific terms used herein have the same meaning as commonly understood by one of ordinary skill in the art to which this invention pertains. The following references can provide a general definition of certain terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

A “neoplasia” can refer to any disease that is caused by, or results in inappropriately high levels of cell division, inappropriately low levels of apoptosis, or both. For example, cancer is an example of a neoplasia. Examples of cancers include leukemias, e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioepdotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, vile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and still more preferably 90%, 95? or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “inhibitory nucleic acid” is meant a single or double-stranded RNA, siRNA (short interfering RNA), shRNA (short hairpin RNA), or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises or corresponds to at least a portion of a target nucleic acid molecule, or an ortholog thereof, or comprises at least a portion of the complementary strand of a target nucleic acid molecule.

By “antisense nucleic acid”, it is meant a non-enzymatic nucleic acid molecule that binds to target RNA by means of RNA-RNA or RNA-DNA interactions and alters the activity of the target RNA (for a review, see Stein et al. 1993; Woolf et al., U.S. Pat. No. 5,849,902). Typically, antisense molecules are complementary to a target sequence along a single contiguous sequence of the anti sense molecule. However, in certain embodiments, an antisense molecule can bind to substrate such that the substrate molecule forms a loop; and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk N A et al., 1999; Delihas N et at, 1997; Aboul-Fadl T, 2005.)

The term “siRNA” refers to small interfering RNA; a siRNA is a double stranded RNA that “corresponds” to or matches a reference or target gene sequence. This matching need not be perfect so long as each strand of the siRNA is capable of binding to at least a portion of the target sequence. siRNAs can be used to inhibit gene expression, see for example Bass, 2001, Nature, 411, 428 429; Elbashir et al., 2001, Nature, 411, 494 498; and Zamore et al., Cell 101:25-33 (2000).

The embodiments described herein are not limiting and one skilled in the art can readily appreciate that specific combinations of the modifications described herein can be tested without undue experimentation toward identifying nucleic acid molecules with improved RNAi activity.

All publications, patents and literature specifically mentioned herein are incorporated by reference in their entirety for all purposes.

It is understood that this invention is not limited to the particular methodology, protocols, materials, and reagents described, as these may vary. It is also to be understood that the terminology used herein is for the purpose of describing particular embodiments only, and is not intended to limit the scope of the present invention. It will be readily apparent to one skilled in the art that varying substitutions and modifications can be made to the description disclosed herein without departing from the scope and spirit of the description, and that those embodiments are within the scope of this description and the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprises,” “comprising”, “containing,” “including”, and “having” can be used interchangeably, and shall be read expansively and without limitation.

Recitation of ranges of values herein are merely intended to serve as a shorthand method of referring individually to each separate value falling within the range, unless otherwise indicated herein, and each separate value is incorporated into the specification as if it were individually recited herein. For Markush groups, those skilled in the art will recognize that this description includes the individual members, as well as subgroups of the members of the Markush group.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

All of the features disclosed in this specification may be combined in any combination. Each feature disclosed in this specification may be replaced by an alternative feature serving the same, equivalent, or similar purpose.

1-22. (canceled)

23. A method for preventing, treating or ameliorating one or more symptoms of a malignant tumor associated with KRAS mutation in a mammal in need thereof, the method consisting of:
administering to the mammal a therapeutically effective amount of a composition comprising one or more RNAi molecules that are active in reducing expression of GST-π,
wherein the composition comprises only one active ingredient and the active ingredient is the RNAi.

24. The method of claim 23, wherein the mammal is a human and the GST-π is a human GST-π.

25. The method of claim 23, wherein the RNAi molecule is a siRNA or shRNA.

26. The method of claim 23, wherein the RNAi molecules comprise a duplex region comprising a nucleotide sequence corresponding to a target sequence of SEQ ID NO:287.

27. The method of claim 23, wherein the RNAi molecule decreases expression of GST-π in the mammal.

28. The method of claim 23, wherein the administration decreases expression of GST-π in the tumor cell by at least 5% for at least 5 days.

29. The method of claim 23, wherein the administration decreases the volume of the malignant tumor in the mammal by at least 5%, or at least 10%, or at least 20%, or at least 30%, or at least 40%, or at least 50%.

30. The method of claim 23, wherein a tumor cell in the mammal comprises an increased level of expression of wild type KRAS protein compared to that in a non-tumor cell of the same tissue.

31. The method of claim 23, wherein the malignant tumor is a carcinoma selected from the group consisting of lung adenocarcinoma, mucinous adenoma, ductal carcinoma of the pancreas, colorectal carcinoma, breast cancer, and fibrosarcoma.
